SEQUENCE LISTING (167 sequences)

SEQ ID NO: 1 - protein, 742 aa, C. thermocellum
MDAWRGFNKGNWCQEIDVRDFIIRNYTPYEGDESFLVGPTDRTRKLWEKVSELLKKERENGGVLDVDTHTISTITSHKPGYIDKELEVIVGLQTDEPLKRAIMPFGGIRMVIKGAEAYGHSVDPQVVEIFTKYRKTHNQGVYDVYTPEMRKAKKAGIITGLPDAYGRGRIIGDYRRVALYGVDRLIAEKEKEMASLERDYIDYETVRDREEISEQIKSLKQLKEMALSYGFDISCPAKDAREAFQWLYFAYLAAVKEQNGAAMSIGRISTFLDIYIERDLKEGKLTEELAQELVDQLVIKLRIVRFLRTPEYEKLFSGDPTWVTESIGGMALDGRTLVTKSSFRFLHTLFNLGHAPEPNLTVLWSVNLPEGFKKYCAKVSIHSSSIQYESDDIMRKHWGDDYGIACCVSAMRIGKQMQFFGARCNLAKALLYAINGGKDEMTGEQIAPMFAPVETEYLDYEDVMKRFDMVLDWVARLYMNTLNIIHYMHDKYAYEALQMALHDKDVFRTMACGIAGLSVVADSLSAIKYAKVKPIRNENNLVVDYEVEGDYPKFGNNDERVDEIAVQVVKMFMNKLRKQRAYRSATPTLSILTITSNVVYGKKTGNTPDGRKAGEPLAPGANPMHGRDINGALAVLNSIAKLPYEYAQDGISYTFSIIPKALGRDEETRINNLKSMLDGYFKQGGHHINVNVFEKETLLDAMEHPEKYPQLTIRVSGYAVNFIKLTREQQLDVINRTIHGKI

SEQ ID NO: 2 - protein, 358 aa, C. thermocellum
VIIYSYKYYKYSFYDNSFGIMKGEEFMSFLEQIIERAKSDVKTIVLPESTDLRVIKAASMIMKKGIAKVVLIGNEKEIKSLAGDIDLEGVMIEDSLNSEKLEDYANTLYELRKSKGMTIEAARETIKDPLYYGVMMVKKGEADGMVAGAVNSTANTLRPALQILKTAPGTKLVSSFFVMVVPNCEYGHNGTFVYADCGLVENPDADQLSEIAISASKSFEMLVGAKPQVAMLSYSSYGSAKSELTEKVIKATQLAKEKAPHLAIDGELQVDAAIVPEVAKSKAKGSSVAGKANVLIFPDLDAGNIAYKLTQRLAKAEAYGPITQGLARPVNDLSRGCSAEDIVGVAAITAVQAQYVKA

SEQ ID NO: 3 - protein, 399 aa, C. thermocellum
MNILVINTGSSSLKYQLIDMTNESVLAKGVCDRIGLEHSFLKHTKTGGETVVIEKDLYNHKLAIQEVISALTDEKIGVIKSMSEISAVGHRIVHGGEKFKESAIIDEDVMKAIRDCVELAPLHNPSNIIGIEACKQILPDVPMVAVFDTAFHQTMPRHAYIYALPYEIYEKYKLRKYGFHGTSHKYVAHRAAQMLGKPIESLKLITCHLGNGASICAVKGGKSVDTSMGFTPLQGLCMGTRSGNVDPAVITYLMEKEKMNINDINNFLNKKSGVLGISGVSSDFRDVQDAAEKGDDRAQLALDIFCYGVRKYIGKYIAVLNGVDAVVFTAGIGENNAYIRREVLKDMDFFGIKIDLDKNEVKGKEADISAPDAKVKTLVIPTNEELEIARETLRLVKNL

SEQ ID NO: 4 - protein, 389 aa, C. thermocellum
MINFVYKNPTKIIFGRGTELKVGEEVRQYSGKVLLHYGGGSIKKTGLYDRVVNSLKQAGVEVVELGGVMPNPRLGLVNEGIKICREKGIDFILAVGGGSAIDSAKAIAVGVPYDGDVWDFFCGKAEPKEALPVGVVLTIPAAGSEASPNSVITREDGLYKRGMYSELIRPVFAIMNPELTYTLPAYQTACGTADIMAHIMERYFTNETHTDLTDRLCEATLKTMIKNVPIALEEPDNYNARAEIMWAGTIAHNGLLGTGRIEDWASHNIEHEISAIYDVAHGAGLAVVFPAWMKYVYKNNLDRFVQFAVRVWNVEMNFDEPERTALEGIERLKKFFKEIGLPVSLKEMNIGDDRLEEMASKCTNGGKATIGNFVKLNREDVYNILKLAV

SEQ ID NO: 5 - protein, 389 aa, C. thermocellum
MKAFNYYAPTEIIFGCGRVQEIGSITAQYGKKALLVTVPEFPEVKELYEKVKKSLRENGVEVVHFDGVIPNPTTDVVTEGANMAKAAGVDVVIGLGGGSSIDTAKAIAVEATHPGTAWDYNCHTPGPTSATLPIIAIGTTAGTGSQCTQCAVITKTSEKDKSAIWHKNIFPKVAIVDPEVTVTMPKSVTAQTGFDAFAHNFEAYLSVKTSPLVEMMAIEAIKMIKEYLPKALENPNDIEARSKMSLADTLGGLTNSNAGVTLPHGLGMQVGGHAPHVSHGQALAIIYPQFTRYTYAWAIEKFAKVGRIFNPALNELSDEEAAKEACVAIDDFLKKIGLWIGFKDVNVTKEQIREIADDGQVLGDYLNNPRVATIDEMYELLMNCYERKE

SEQ ID NO: 6 - protein, 873 aa, C. thermocellum
MTKIANKYEVIDNVEKLEKALKRLREAQSVYATYTQEQVDKIFFEAAMAANKMRIPLAKMAVEETGMGVVEDKVIKNHYASEYIYNAYKNTKTCGVIEEDPAFGIKKIAEPLGVIAAVIPTTNPTSTAIFKTLIALKTRNAIIISPHPRAKNSTIEAAKIVLEAAVKAGAPEGIIGWIDVPSLELTNLVMREADVILATGGPGLVKAAYSSGKPAIGVGAGNTPAIIDDSADIVLAVNSIIHSKTFDNGMICASEQSVIVLDGVYKEVKKEFEKRGCYFLNEDETEKVRKTIIINGALNAKIVGQKAHTIANLAGFEVPETTKILIGEVTSVDISEEFAHEKLCPVLAMYRAKDFDDALDKAERLVADGGFGHTSSLYIDTVTQKEKLQKFSERMKTCRILVNTPSSQGGIGDLYNFKLAPSLTLGCGSWGGNSVSDNVGVKHLLNIKTVAERRENMLWFRTPEKIYIKRGCLPVALDELKNVMGKKKAFIVTDNFLYNNGYTKPITDKLDEMGIVHKTFFDVSPDPSLASAKAGAAEMLAFQPDTIIAVGGGSAMDAAKIMWVMYEHPEVDFMDMAMRFMDIRKRVYTFPKMGQKAYFIAIPTSAGTGSEVTPFAVITDEKTGIKYPLADYELLPDMAIVDADMMMNAPKGLTAASGIDALTHALEAYVSMLATDYTDSLALRAIKMIFEYLPRAYENGASDPVAREKMANAATIAGMAFANAFLGVCHSMAHKLGAFYHLPHGVANALMINEVIRFNSSEAPTKMGTFPQYDHPRTLERYAEIADYIGLKGKNNEEKVENLIKAIDELKEKVGIRKTIKDYDIDEKEFLDRLDEMVEQAFDDQCTGTNPRYPLMNEIRQMYLNAYYGGAKK

SEQ ID NO: 7 - protein, 347 aa, C. thermocellum
MKGKMKVCVLTGKEKLEWVERDIPQPGRGELQIKLKHVGVCGSDLHFYKEGRLANWELDGPLALGHEPGGIVSAIGEGVEGFEIGDKVALEPGVPCGECEDCRKGHYNLCKHIKFMAIPHEKDGVFAEYCVHSASMCYKLPENVDTMEGGLMEPLSVALHATELSNAKIGETAIVLGSGCIGLCTVMALKARGVSEIYVTDVVDKRLEKALEVGATRVFNSQREDIVEFAKTLPGGGADQVYECAGSRVTTLQTCKLIKRAGKVTLVGVSPEPVLELDIATLNAMEGTVYSVYRYRNMYPIAIAAVSSGVIPLKKIVSHVFDFKDCIEAIEYSTNHKDEVIKSVIKF

SEQ ID NO: 8 - protein, 368 aa, C. thermocellum
MNFKFKIGTKVFFGKECVKENKAVFKDFRKRALLVTGKNSAKASGAFSDVVEVLEEYGIDYEIYDRVANNPSLENVKEGGEAARKFDADFIIGIGGGSPLDASKAVAVLATNDIEPVDLYKNVFENKPLPIIAIPTTAGTGSEVTPYSILTRDDMKTKKSFGNEDTFPAVAFIDARYTESMSYETTVDTALDAFTHALEGYLGRRSTPVSDILAVEAIRIFGECLENLLNNKFDYDVREKLLYMSMLGGMVISHTGTTIIHGMGYSLTYFKDIPHGRANGMLVREYLKYNYEAAKEKTDNVLRLLKVPSIDAFGEIIDRLIPQKPVLTKEEIELYASLAMKQNSTLSNARTVVKEDMEEIFKNTFGKG

SEQ ID NO: 9 - protein, 131 aa, C. thermocellum
MNIALIAHDKKKELMASFCIAYRSILKNHTLFATGTTGAIIVEATGLNVHRFLPGVMGEQQISARAAYNELDLVIFFRDPISAKSDEPDIHSLLRECDINNIPFATNLGTAEMLIKGLERGDLDWRELIKK

SEQ ID NO: 10 - protein, 315 aa, C. thermocellum
LKYCKLGNTGLEVSKLCFGGLIIGPLQANLPPETGAEIILKSFELGVNFIDTAELYGTYSHIGKALKKTNKNIVVATKSYAYSAEGAKESLEKARKEMDIDVIDIFMLHEQESRLTLKGHREALEYYISMKEKGIIKAVGVSTHNVEVVEACCEMPEVDVIHPIVNKAGIGIGDGTIDDMLKAVEKAYSVGKGIYSMKPLGGGNLIKSYKEAMDFVLNIPYIHSIAVGMQSIEEVVMNVCIFEGKEVPQDVQKSLENKKRHLHIDWWCEGCGKCVERCKQKALKLVDGKAKVEEEKCVLCSYCASVCPVFAIKVS

SEQ ID NO: 11 - protein, 376 aa, C. thermocellum
MQYRGLGKTGVKVSALGFGAMRLPQININGNTRVDEEKSIEMIHRAFELGVNYIDTAPGYCNGESEVVVGKALKGWRDKIYLSTKNPIENASGDDWRKRLENSLKKLDTDYIDFYHMWGINWETYETKIDVKGGPLEAARKAKEEGLIRHISFSFHDKPENLIKLIDTGNFETVLCQYNLLDRSNEKAIAHAKRKGLGVIIMGPVGGGKLGEPSETIKKLLPKKTVSCAEIALRFVLANPNVDCALSGMSTIEMVEENVRVASNDTPLTKEELEMIRASMEENKRMEDLYCTGCNYCMPCPVGVNIPLNFQLMNYHRVYKITDYARGQYSQIGKVEWYKGKPAHECIECGVCETKCPQKLEIRKQLKETARVLSVK

SEQ ID NO: 12 - protein, 315 aa, C. thermocellum
MKYRKMGRTGLYISEISLGSWLTYGNSTDKETAVKVIDTAYSLGINYFDTANVYANGRAEVIVGEALKKYPRESYILATKAFWPMGTGPNDKGLSRKHVFEQVHASLKRLNVDYIDIFYCHRYDPETPLEETLRTIDDLLRQGKILYVGVSEWTAAQMAQALHIADRYLLDRIVVNQPQYNMFHRYIEKEIIPFGEKNGISQIVFSPLAQGVLTGKYKPGGNIPRDSRAADPNSNMYIGQFLKEDKLLKVEKLKAVADEMGITLSQLAIAWVLRQPNVTSALIGASKPEQVEENVKASGINLSDEILNKIEAILQ

SEQ ID NO: 13 - protein, 251 aa, C. thermocellum
MSRKVIAAGNWKMNKTPKEAVEFVQALKGRVADADTEVVVGVPFVCLPGVVEAAKGSNIKVAAQNMHWEEKGAFTGEVSGPMLAELGVDYVIIGHSERRQYFGETDETVNKKVHAAFKYGLKPIICVGESLTQREQGVTAELVRYQVKIALLGLSAEQVKEAVIAYEPIWAIGTGKTATNEQAEEVCGIIRECIKELYGQDVAEAIRIQYGGSVNAANAAELFNMPNIDGGLVGGASLKLDDFEKIAKYNK

SEQ ID NO: 14 - protein, 192 aa, C. thermocellum
MGKVVEIRWHGRGGQGAKTASLLLADAAFNTGKYIQGFPEYGPERMGAPITAYNRISDEKLTIHSNIYEPDYVVVVDDTLLTSVDVTAGLKEDGAIIVNTPKTPDEIRPLLKGYKGKVCTIDARKISIETLGKYFPNTPMLGAVVKVSKIMDEEEFLKDMVESFKHKFANKPEVVEGNIKALERSMQEVKGL

SEQ ID NO: 15 - protein, 101 aa, C. thermocellum
MSKELRDVKPDVTWKEITSGGVIDSPGNAHLFKTGDWRSMKPVWNEEKCKQCLLCNPVCPDSSIMVSEEGKMTGIDYDHCKGCGICSKVCPFKAIDFVEEV

SEQ ID NO: 16 - protein, 394 aa, C. thermocellum
MGIRERLSGNEATAIAMRQINPDVVAAFPITPSTEIPQYFSSYVADGLVDTEFVAVESEHSAMSACIGAQAAGARAMTATSANGLAYMWEALYIAASMRLPIVLAAVNRALSGPINIHNDHSDTMGARDSGWIQLYSENNQEAYDNMLMAHRIGEHPDVMLPVMVCQDGFITSHAIENIELVEDEKVKAFVGEYKPTHYLLDRENPISVGPLDLQMHYFEHKRQQAQAMENAKKVILEVAEEFYKLTGRKYGFFEEYKTDDADVAIVVMNSTAGTVKYVIDEYRAKGKKVGLIKPRVFRPFPVDELAQALSKFKAVAVMDKADSFNAAGGPLFTEVTSALFTKGVFGPKVINYKFGLGGRDVKVDDIEVVCEKLLEIASTGKVDSVYNYLGVRE

SEQ ID NO: 17 - protein, 311 aa, C. thermocellum
MAYNLKEVAKKPERLTGGHRMCAGCGAPIVVRQVLKALKPEDHAVISAATGCLEVSTFIYPYTAWKDSFIHSAFENTGATISGAEAAYKVLKKKGKIEGETKFIAFGGDGGTYDIGLQALSGAMERGHDMVYVCYDNGAYMNTGIQRSSATPKYADTTTSPVGKKIPGKMQPRKDLTEVLVNHRIPYVAQTAPFGNMKDLYEKAEKAIYTPGPAFLNVLAPCPRGWRYNTPDLMELSKLAVETCFWPLYEVIDGKYIINYKPKEKVPVKEFLKLQGRFKHLFKAGNEYMLEEIQKEVDLRWERLLKLAGEA

SEQ ID NO: 18 - protein, 317 aa, C. thermocellum
MNNNKVIKKVTVVGAGFVGSTTAYTLMLSGLISEIVLIDINAKKADGEVMDLNHGMPFVRPVEIYRGDYKDCAGSDIVIITAGANQKEGETRIDLVKRNTEVFKNIINEIVKYNNDCILLVVTNPVDILTYVTYKLSGFPKNKVIGSGTVLDTARFRYLLSEHVKVDARNVHAYIIGEHGDTEVAAWSLANIAGIPMDRYCDECHQCEEQISRNKIYESVKNAAYEIIRNKGATYYAVALAVRRIVEAIVRNENSILTVSSLLEGQYGLSDVCLSVPTIVGVNGIEEILNVPFNDEEIQLLRKSGNTLKEIIKTLDI

SEQ ID NO: 19 - protein, 81 aa, C. thermocellum
MKVSICIGSSCHLKGAKQIVEQLQSLVADYNLKEKVELGGAFCMKNCVNGVSVTVDDKLFSVTPENVKSFFETEILKKLED

SEQ ID NO: 20 - protein, 556 aa, C. thermocellum
MTECLQTKKSNCKNCYKCIRHCPVKSLKFTDGQAHIVRDECVLCGECYVVCPQNAKQIRSDVEKAKQLVLKYDVYASIAPSFVAWFHNKSIHDMEQALIKLGFKGADETAKGAYIVKKQYEKMIEEKKSKIIISSCCHTVNTLIQRHYTGAIQYLADVVSPMLAHAQMLKKEHKGAKVVFIGPCISKKDEAEKYKGYVELVLTFDELDEWLKSENITIESNRGSSKEGRTRSFPVSGGIISSMDKDLGYHYMVVDGMENCINALENIERGEIDNCFIEMSACRGSCINGPPARRKSNNIVGAILAVNKNTGAKDFSVPMPEPEKLKKEFRFEGVHKIMPGGTAIEEILKKMGKTSIEHELNCGSCGYDTCRDKAVAVLNGKADLTMCLPYLKEKAESFSDAIIKNTPNGVIVLNEDLEIQQINNSAKRILNLSPSTDLLGSPVSRILDPIDYILALREGKNCYYKRKYFAEYKKYVDETIIYDKEYHVIIIIMRDVTEEEKIKALKNKQSEAAIEIADKVVEKQMRVVQEIALLLGETAAETKIALTKLKETMEDE

SEQ ID NO: 21 - protein, 389 aa, C. thermocellum
MNDLCVDLGYKSLNKFGEQLCGDMIQVVKDDDTTILVLADGLGSGVKANILSTLTSKIISTMIAAHMGIEECVNTIMSTLPVCKVRGIAYSTFTIIKITNNTYAEIIQYDNPLVILLRNGKKYDYPTQTKIISGKKIVESKIRLNCDDVFVVMSDGAIYAGVGQTLNYGWQRENIIEFIESHYDKSLSANALTSLLIDTCNNLYANMPGDDTTIAAIKIRKRQVVNLMFGPPQNPEDVHNMMSLFFAKQGRHIVCGGTTSTLAAKFLGKELETTIDYIDPRIPPIARIEGVDLVTEGVLTISRVLEYAKDYIGKNILYNEWHSKNDGASIIARMLFEEATDINFYVGKAINPAHQNPNLPIGFNIKMQLVEELSKILKQMGKTINLSYF

SEQ ID NO: 22 - protein, 165 aa, C. thermocellum
MSVTMSEAFDYSMIDNILSEHGTSETAIIAILQSIQEEYHYIPKEVFPYLSKKLKVSEARIFSVATFYENFSLEPKGKYIIKVCDGTACHVRKSIPIIERLRKELGLSGTKPTTDDLMFTVETVSCLGACGLAPVITVNDKVYAEMTPDKASELIKQLREGDADA

SEQ ID NO: 23 - protein, 624 aa, C. thermocellum
MLKNREELRKAREMYSRYLKAEKRRVLVCAGTGCVSGGSMEIFERLSELVSKRGMDCQVELKEEPHDNTIGMKKSGCHGFCEMGPLVRIEPEGYLYTKVKLEDCEEIVDRTIVAGEHIERLAYKQNGVVYKKQDEIPFYKKQTRLVLEHCGQIDSTSITEYLATGGYYALEKALFDMTGDEIINEITEANLRGRGGGGFPAGRKWAQVKRQNAKQKYVVCNGDEGDPGAFMDRSIMEGDPHRMIEGMIIAGIACGASEGYIYVRAEYPLAVSRLKRAIEQAKEFGLLGENILGSNFSFNIHINRGAGAFVCGEGSALTASIEGKRGMPRVKPPRTVEQGLFDMPTVLNNVETFANVPLIIKNGADWYKSIGTEKSPGTKAFALTGNIENTGLIEIPMGTTLREVIFDIGGGMRNGADFKAVQIGGPSGGCLSEKDLDLPLDFDSLKKAGAMIGSGGLVVMDSNTCMVEVARFFMNFTQNESCGKCVPCREGTKRMLEILERIVEGNGQDGDIELLLELADTISATALCGLGKAAAFPVVSTIKNFREEYEAHIYDKRCPTGNCQKLKTITIDASLCKGCSKCARSCPVGAITGKVKEPFVIDQSKCIKCGACIETCAFHAILEG

SEQ ID NO: 24 - protein, 566 aa, C. thermocellum
MDNREYMLIDGIPVEINGEKNLLELIRKAGIKLPTFCYHSELSVYGACRMCMVENEWGGLDAACSTPPRAGMSIKTNTERLQKYRKMILELLLANHCRDCTTCNNNGKCKLQDLAMRYNISHIRFPNTASNPDVDDSSLCITRDRSKCILCGDCVRVCNEVQNVGAIDFAYRGSKMTISTVFDKPIFESNCVGCGQCALACPTGAIVVKDDTQKVWKEIYDKNTRVSVQIAPAVRVALGKELGLNDGENAIGKIVAALRRMGFDDIFDTSTGADLTVLEESAELLRRIREGKNDMPLFTSCCPAWVNYCEKFYPELLPHVSTCRSPMQMFASIIKEEYSTSSKRLVHVAVMPCTAKKFEAARKEFKVNGVPNVDYVLTTQELVRMIKESGIVFSELEPEAIDMPFGTYTGAGVIFGVSGGVTEAVLRRVVSDKSPTSFRSLAYTGVRGMNGVKEASVMYGDRKLKVAVVSGLKNAGDLIERIKAGEHYDLVEVMACPGGCINGGGQPFVQSEEREKRGKGLYSADKLCNIKSSEENPLMMTLYKGILKGRVHELLHVDYASKKEAK

SEQ ID NO: 25 - protein, 81 aa, C. thermocellum
MLEIKICVGSSCHLKGSYNVINEFQHLIEEKALHDKIDIKATFCMKQCQKNGVAVEVNNEIFGVLPEAAEEFFKNVILPKV

SEQ ID NO: 26 - protein, 128 aa, C. thermocellum
MSFFTMTKTLIKSIFHGPYTVRYPLEKKEPFPASRGRIEINIQDCIFCGLCARRCPTGAINVEKPESRWSINRLRCIQCGYCSEVCPKKCLKMNNMYPAPSFENIEDVYQNARVPDNKENNRNIAGAC

SEQ ID NO: 27 - protein, 359 aa, C. thermocellum
MGKKTVIPFGPQHPVLPEPIHLDLVLEDETVVEAIPSIGYIHRGLEKLVEKKDYQQFVYVAERICGICSFMHGMGYCMSIENIMGVQIPERAEFLRTIWAELSRIHSHMLWLGLLADALGFESLFMHSWRLREQILDIFEETTGGRVIFSVCDIGGVRRDIDSEMLKKINSILDGFEKEFSEITKVFLNDSSVKLRTQGLGVLSREEAFELGAVGPMARASGIDIDMRKSGYAAYGKLKIEPVVETAGDCYARTSVRIREVFQSIDLIRQCISLIPDGEIKVKIVGNPSGEYFTRLEQPRGEVLYYVKANGTKFLERFRVRTPTFANIPALLHTLKGCQLADVPVLILTIDPCISCTER

SEQ ID NO: 28 - protein, 119 aa, C. thermocellum
MAQQTINTISPNELLAYALRLKNANYRLVAISCTNAENGVEMSYSFDSGSDFTNLRITVAPGDEIESISSIYSYSFLYENEIKELFGVNITGISPDYKDKLYRISVKTPFNMKEGDKNG

SEQ ID NO: 29 - protein, 145 aa, C. thermocellum
MNFSKKSPWILHYDGSSCNGCDIEVLACLTPLYDIERFGVINTGNPKHADILLITGSINEQNKSVVKQLYEQMADPKVVVAVGICAATGGIFSECYNVSGGVDKIIPVDVYVPGCAARPEAIIDGVVKALGILEERQKYARKKDK

SEQ ID NO: 30 - protein, 291 aa, C. thermocellum
MSQIIRLVLYIIAIIIVAPLLGGLLTGIDRVITARMQGRKGPSVLQPFYDVLKLFQKESIEVNTMHRFFVYISLIFVIFTTVIMLLGGDILLALFALTLGSIFFVLGGYASNSPYSTIGSERELLQMMAFEPMLLLAAIGLYYGDKSFFIKDIVTARIPSIVYLPGVFLGLLYVLTFKLRKSPFDLSMSHHGHQEIVQGITTEYSGKDLAIIQITHWYETIIALALVYLFFAFRSPFSHVIAILACIIAFLLEIVVDNAFARAKWEFALKSTWIVTGVLASVNLIILSFFR

SEQ ID NO: 31 - protein, 636 aa, C. thermocellum
MNAILILILFPLLASVTVLSVRKDAIRNIIVRIFAFITGILTLFVVCRYFKDGISLSIENRNIIDMTISLAEVLIAAYIIFTGIKNKKFIVSIFAAVQTALILWFEFTQKHGINVHSDIVFDRLSAVMVLIVGCIGSLILIYTVGYMKWYHIHHEGYKERKSFFFSVIFLFLFAMFGLIFSNNLIWMYFCWELTTLCSYLLIGYTRTPEAVNNSFHALAINLGGGLAFASAMVYIGTNFKTLELSALTAMKLELAVLIPVFLLCIAALTKSAQMPFSSWLLGAMVAPTPSSALLHSATMVKAGVYLLIRLAPLLAGTTIGKVIALLGAVTFLASSIIAISKSDAKKILAYSTISNLGLIVTCAAIGTQESLWAAILLLIFHSISKSLLFLTGGSVEHQIGSRNVEDMDILLQVSRRLSVYMIVGIAGMFLAPFGMLISKWVAMKAFIDSKNILTVIILGYGSATTLFYWTKWMGKLVANANRKDHIKHTFHIDEEIPIFIHAVLVVLSCFTFPLVSRYVLVPYLSGLFGPDVPIPIGTSDVNIMLIMLSMLLILPISFIPIYKSDRRRIVPIYMAGENTGDNESFYGAFDEKRKVELHNWYMKNFFSVKKLTFWSNLLCAVVILVGVVLLIGGITK

SEQ ID NO: 32 - protein, 582 aa, C. thermocellum
MQMVNVTIDNCKIQVPANYTVLEAAKQANIDIPTLCFLKDINEVGACRMCVVEVKGARSLQAACVYPVSEGLEVYTQTPAVREARKVTLELILSNHEKKCLTCVRSENCELQRLAKDLNVKDIRFEGEMSNLPIDDLSPSVVRDPNKCVLCRRCVSMCKNVQTVGAIDVTERGFRTTVSTAFNKPLSEVPCVNCGQCINVCPVGALREKDDIDKVWEALANPELHVVVQTAPAVRVALGEEFGMPIGSRVTGKMVAALSRLGFKKVFDTDTAADLTIMEEGTELINRIKNGGKLPLITSCSPGWIKFCEHNYPEFLDNLSSCKSPHEMFGAVLKSYYAQKNGIDPSKVFVVSIMPCTAKKFEAQRPELSSTGYPDVDVVLTTRELARMIKETGIDFNSLPDKQFDDPMGEASGAGVIFGATGGVMEAAIRTVGELLSGKPADKIEYTEVRGLDGIKEASIELDGFTLKAAVAHGLGNARKLLDKIKAGEADYHFIEIMACPGGCINGGGQPIQPSSVRNWKDIRCERAKAIYEEDESLPIRKSHENPKIKMLYEEFFGEPGSHKAHELLHTHYEKRENYPVK

SEQ ID NO: 33 - protein, 566 aa, C. thermocellum; identical to SEQ ID NO: 24

SEQ ID NO: 34 - protein, 644 aa, C. thermocellum
MDSFLMKGYIKEANIDYSCSRGSMEDLPKWEFREIPKVPRAVMPSLSLEERKNNFNEVELGLSEEVARKEARRCLKCGCSARFTCDLRKEASNHGIVYEEPIHDRPYIPKVDDHPFIVRDHNKCISCGRCIAACAEIEGPGVLTFYMKNGRQLVGTKSGLPLRDTDCVSCGQCVTACPCAALDYRRERGKVVRAINDPKKTVVGFVAPAVRSLISNTFGVSYEEASPFMAGLLKKLGFDKVFDFTFAADLTIVEETTEFLSRIQNKGVMPQFTSCCPGWINFVEKRYPEIIPHLSTCKSPQMMMGATVKNHYAKLMGINKEDLFVVSIVPCLAKKYEAARPEFIHDGIRDVDAVLTTTEMLEMMELADIKPSEVVPQEFDEPYKQVSGAGILFGASGGVAEAALRMAVEKLTGKVLTDHLEFEEIRGFEGVKESTIDVNGTKVRVAVVSGLKNAEPIIEKILNGVDVGYDLIEVMACPGGCICGAGHPVPEKIDSLEKRQQVLVNIDKVSKYRKSQENPDILRLYNEFYGEPNSPLAHELLHTHYTPKHGDSTCSPERKKGTAAFDVQEFTICMCESCMEKGAENLYNDLSSKIRLFKMDPFVQIKRIRLKETHPGKGVYIALNGKQIEEPMLSGNIPDESESE

SEQ ID NO: 35 - protein, 493 aa, C. thermocellum
MKTLENHNRIKVTVNGREIEVYDNLTILQALLQEDIHIPHLCYDIRLERSNGNCGLCVVTLISPDGERDVKACQTPIKEGMVICTNTPKLENYRKIRLEQLLSDHNADCVAPCVMTCPANIDIQSYLRHVGNGDFEAAIRVIKERNPFPIVCGRVCPHTCESQCRRNLVDAPVAINYVKRFAADWDMARPEPWTPEKKPPTGKKIAIVGAGPSGLSAAYYSAIKGHDVTVFERQPHPGGMMRYGIPEYRLPKAILDKEIEMIKKLGVKIMTEKALGIHIRLEDLSKDFDAVYLAIGSWQATPMHIEGEKLDGVWAGINYLEQVAKNVDIPLGDNVVVIGGGNTAIDCARTALRKGAKSVKLVYRCTREEMPAAPYEVEEAIHEGVEMIFLMAPTKIIVKDGKKKLVCIRMQLGEPDRSGRRRPVPIEGSEVEIDADTIIGAIGQSTNTQFLYNDLPVKLNKWGDIEVNGKTLQTSEYNIFAGGDCVTGPATVI

SEQ ID NO: 36 - protein, 309 aa, C. thermocellum
MPLVTSTEMFKKAYEGKYAIGAFNVNNMEIIQGITEAAKEVNAPLILQVSAGARKYANHTYLVKLVEAAVEETGLPICLHLDHGDSFELCKSCIDGGFTSVMIDGSHLPFEENIKLTKQVVDYAHSKGVVVEGELGRLAGIEDDVNVSEADAAFTDPDQAEEFVKRTGVDSLAIAIGTSHGAYKFKGEAKLRFDILEEIEKRLPGFPIVLHGASSVIPEYVDMINKYGGDMPGAKGVPEDMLRKAASMAVCKINIDSDLRLAMTATIRKYFAENPSHFDPRQYLGPARNAIKELVKHKIVNVLGCDGKA

SEQ ID NO: 37 - protein, 327 aa, C. thermocellum
MDIQLKKSGIGVKEKKSKNHLLYSIKQNLFAYAMLIPTFVCMMCIHFIPMLQGIYLSLLDLNQLTMTKFLNAPFIGLKNYYEILFDEKSLIRRGFWFALRNTAIYTVVVTFATFALGIILAMLVNREFKGRGIVRTALLMPWVVPSYVVGMTWGFLWRQDSGLINIILCDILHILPEKPYWLVGSNQIWAIIIPTIWRGLPLSMILMLAGLQSISPDYYEAADIDGANGWQKFWHITLPLLKPILAINVMFSLISNIYSFNIVSMMFGNGAGIPGEWGDLLMTYIQRNTFQMWRFGPGAAALMIVMFFVLGIVALWYTLFKDDLVVK

SEQ ID NO: 38 - protein, 404 aa, C. thermocellum
VDKFTKLDLNSITSNNRMNIFNCILEAKEINRAVIAKKVGLSIPAVMSITDDLIQKGIIYVIGKGKSSGGKRPELLAVVPDRFFFVGVDVGRTSVRVVVMNNCRDVVYKVSKPTESVEPDELINQITEMTMESINESKFPLDRVVGIGVAMPGLIERGTGRVIFSPNFGWNNIALQDELKKHLPFNVLVENANRALVIGEIKNTQPNPTSCIVGVNLGYGIGSAIVLPNGLYYGVSGTSGEIGHIIVENHGSYCSCGNYGCIESIASGEAIAREARIAIANKIQSSVFEKCEGDLKKIDAKMVFDAAKEGDHLAQSIVEKAADYIGKGLAITINMLDPEQIILCGGLTLSGDFFIDMIKKAVSKYQMRYAGGNVKIVVGKSGLYATAIGGAWIVANNIDFLSSN

SEQ ID NO: 39 - protein, 317 aa, C. thermocellum
MYYIGIDLGGTNIAVGLVNEEGKILHKDSVPTLRERPYQEIIKDMAMLTLKVIKDADVSIDQVKSIGVGSPGTPNCKDGILIYNNNLNFRNVPIRSEIQKYIDLPVYLDNDANCAALAESVAGAAKGANTSVTITLGTGIGGGVVIDGKIYSGFNYAGGELGHTVLMMDGEPCTCGRKGCWEAYASATALIRQARKAAEANPDSLINKLVGGDLSKIDAKIPFDAAKQGDKTGEMVVQQYIRYIAEGLINMINIFMPEVLVIGGGVCKEGEYLLKPLRELIKQGVYSKEDIPQTELRTAQMGNDAGIIGAAMLGKEC

SEQ ID NO: 40 - protein, 448 aa, C. thermocellum
MERIKFDYSKALPFVSEREVAYFENFVRSAHDMLHNKTGAGNDFVGWVDLPVNYDREEFARIKAAAEKIKSDSDALVVIGIGGSYLGARAAIEMLSHSFHNLMPKSKRNAPEIYFVGNNISSTYIADLLEVIEGKEISVNVISKSGTTTEPAIAFRIFKEYMENKYGKDGASKRIYATTDKEKGALRKLATEEGYETFVVPDDIGGRFSVLTAVGLLPIAVAGIDIDSMMKGAADARELYSNPNLMENDCYKYAAVRNALYRKNKTIEIMVNYEPSLHYFTEWWKQLYGESEGKDQKGIFPAGVDFTTDLHSMGQYIQDGLRNIFETVIRVEKPRKNIVIKEEKDNLDGLNFIAGKDVDYVNKKAMEGTVLAHTDGGVPNLVVTVPELSAYYFGNMVYFFEKACGISGYLLGVNPFDQPGVEAYKKNMFALLGKPGYEEQRKKLEERL

SEQ ID NO: 41 - protein, 324 aa, C. thermocellum
MSSVRTIGVLTSGGDAPGMNAAIRSVVRTGLYYGFKVLGIRKGFNGLINGDIEELTARSVGDIIHRGGTILQTARSPQFKTEEGLKKAMSMAKVFGIDALVVIGGDGSYRGARDISKLGLNVIGIPGTIDNDIGCTDYTIGFDTAMNTVQDAIDKIRDTAYSHERCSVLEVMGRHAGYIAVNVSISGGAEAVVLPEKPFDMDTDVIKPIIEGRNRGKKHYLVIVAEGGEGKAIEIAKEITEKTGIEARATILGHIQRGGSPTVYDRVMASQMGAKAVEVLMENKRNRVIVFKDNQIGDMDLEEALQVKKTISEDLIQLSKILAL

SEQ ID NO: 42 - protein, 327 aa, T. saccharolyticum
MSYIPNENRYEKMIYRRCGRSGIMLPAISLGLWHNFGGYDVFENMREMVKKAFDLGITHFDLANNYGPPPGSAEENFGKILRTDLRGYRDELLISTKAGYTMWPGPYGDWGSRKYLLSSLDQSLKRMGIDYVDIFYSHRRDPNTPLEETMSALAQAVRQGKALYVGISNYNAEDTKKAAEILRQLGTPLLINQPSYSMFNRWIEDGLTDVLEEEGVGSIAFSPLAQGLLTDKYLNGVPDDSRAVRKNTSLRGNLTEENINKVRELKKIADKRGQSIAQMALAWDLRKVTSVIIGASRVSQIEENVKALDNLEFSHEELKQIDEILSK

SEQ ID NO: 43 - protein, 131 aa, T. saccharolyticum
LNIALIAHDMKKSIMVDFAIAYKEILKKCNIYATGATGQLVEEATGIKVNKFLPGPMGGDQQIGAMIAENNMDLVIFLRDPLTAQPHEPDILALLRVCDVHSIPLATNLATAEVLIKGLDAGFLEWRDAVK

SEQ ID NO: 44 - protein, 248 aa, T. saccharolyticum
LRRPIIAGNWKMYMTPSEAVNLVNELKPLVSGAEAEVVVIPPFVDLVDVKKAIDASNIKLGAQNMHWEEKGAFTGEVSPIMLKEIGVEYVVIGHSERRQYFAETDETVNKKVKSALSHGLKPIVCVGESLSQREAGEAFNVVREQTKKALDGIKSEDVLNVVIAYEPIWAIGTGKTATSKDANDVIKVIRETIADIYSIDIANEVRIQYGGSVKPDNAKELMSESDIDGALVGGASLKAQDFAKIVNY

SEQ ID NO: 45 - protein, 394 aa, T. saccharolyticum
MYMKTNFTYFMPTEIFGPGTLGKLATVKLPGKKALLVIGSGNSMRRHGYLDRVVNYLKQNGVDYVVYDKILPNPIAEHVAEGAKVAKDNGCDFVIGLGGGSTIDSSKAIAVMAKNPGDYWDYVSGGSGKGMEVKNGALPIVAIPTTAGTGTESDPWAVVTKTETNEKIGFGCKYTYPTLSIVDPELMVSIPPKFTAYQGMDAFFHSVEGYLATVNQPGSDVLALQSISLITENLPKAVADGNNMEARTALAWASTAAGIVESLSSCISHHSLEHALSAYHPEIPHGAGLIMLSVSYFSFMASKAPERFVDIAKAMGEEIVGNTVEEQAMCFINGLKKLIRNIGMEDLSLSSFGVTEDEATKLAKNAMDTMGGLFNVDPYKLSLDEVVSIYKNCF

SEQ ID NO: 46 - protein, 343 aa, T. saccharolyticum
VDDKKVFDHLFILTDDTGMMQHSVGSVPDPKYGYTTDDNGRALIACAMMYEKYKDDAYINLIKKYLSFLMYAQEDDGRFRNFMSFDRKFIDEDFSEDCFGRCMWALGYLINSNIDERVKLPAYKMIEKSLLLVDTLNYIRGKAYTLIGLYYIYNSFKNLDKDFVRKKMDKLAHDIVEEYEKNSSEDWQWFEDVVSYDNGVIPLSLLKYFSIAKDEEVLDIALKTIDFLDSVCFKNGYFKAVGCKGWYRKGKDIAEYDEQPVEAYTMALMYIEAYKLTGDEKYKKRAIDCDKWFYGKNSKGLSLYDEDSGGCSDGITEDGVNSNEGAESLISIMISHCAIDQLK

SEQ ID NO: 47 - protein, 350 aa, T. saccharolyticum
MKTSELLAMVVEKGASDLHITVGVPPVLRINGQLIKLNLPQLTPQDTEEITKDLLSSDELKKLEDMGDIDLSYSVKGLGRFRINAYKQRGTYSLAIRSVALRIPTIDELGLPEVIKELALKTRGLIIVTGPTGSGKSTTLASMIDLINEERNCHILTLEDPIEYLHKHKKSIVNQREIGHDAASYASALRAALREDPDVILVGEMRDLETIQIAITAAETGHLVLSTLHTIGSAKTIDRIIDVFPPHQQQQIKVQLSNVLEGIVSQQLLPKIDNSGRVVAVEVMIATPAIRNLIREGKSFQIQSMVQTGNKFGMVTMDMWISQLLKRNLISMDDALTYCVDRENFSRLVV

SEQ ID NO: 48 - protein, 564 aa, T. saccharolyticum
MIKKKLGDLLVEVGLLDESQLNNAIKIQKKTGEKLGKILVKEGYLTEEQIIEALEFQLGIPHIDMKKVFIDANVAKLIPESMAKRHVAIPIKKENNSIFVAMADPLNIFAIDDIKLVTKLDVKPLIASEDGILKAIDRVFGKEEAERAVQDFKKELSHDSAEDDGNLLRDISEDEINNAPAVRLVNSIIEQAVKNRASDVHIEPTENDLRIRFRIDGELHEAMRVFKSTQGPVITRIKIMANMNIAERRIPQDGKIEMNAGGKNIDIRVSSLPTIYGEKLVLRILDKSGYIITKDKLGLGNDDLKLFDNLLKHPNGIILLTGPTGSGKTTTLYAMLNELNKPDKNIITVEDPVEYTLEGLNQVQVNEKAGLTFASALRSILRQDPDIIMIGEIRDRETAEIAIRSSITGHLVLSTLHTNDSAGAITRLIDMGIEPYLVSSSVVGVIAQRLARKICDNCKIEYDASKREKIILGIDADESLKLYRSKGCAVCNKTGYRGRVPIYEIMMMTPKIKELTNEKAPADVILNEAVSNGMSTLKESAKKLVLSGVTTVDEMLRLTYDDAY

SEQ ID NO: 49 - protein, 1175 aa, T. saccharolyticum
MSKVMKTMDGNTAAAHVAYAFTEVAAIYPITPSSPMAEHVDEWSAHGRKNLFGQEVKVIEMQSEAGAAGAVHGSLAAGALTTTFTASQGLLLMIPNMYKIAGELLPGVFHVSARALASHALSIFGDHQDVMACRQTGFALLASGSVQEVMDLGSVAHLAAIKGRVPFLHFFDGFRTSHEYQKIEVMDYEDLRKLLDMDAVREFKKRALNPEHPVTRGTAQNPDIYFQEREASNRYYNAVPEIVEEYMKEISKITGREYKLFNYYGAPDAERIVIAMGSVTETIEETIDYLLKKGEKVGVVKVHLYRPFSFKHFMDAIPKTVKKIAVLDRTKEAGAFGEPLYEDVRAAFYDSEMKPIIVGGRYGLGSKDTTPAQIVAVFDNLKSDTPKNNFTIGIVDDVTYTSLPVGEEIETTAEGTISCKFWGFGSDGTVGANKSAIQIIGDNTDMYAQAYFSYDSKKSGGVTISHLRFGKKPIRSTYLINNADFVACHKQAYVYNYDVLAGLKKGGTFLLNCTWKPEELDEKLPASMKRYIAKNNINFYIINAVDIAKELGLGARINMIMQSAFFKLANIIPIDEAVKHLKDAIVKSYGHKGEKIVNMNYAAVDRGIDALVKVDVPASWANAEDEAKVERNVPDFIKNIADVMNRQEGDKLPVSAFVGMEDGTFPMGTAAYEKRGIAVDVPEWQIDNCIQCNQCAYVCPHAAIRPFLLNEEEVKNAPEGFTSKKAIGKGLEGLNFRIQVSVLDCTGCGVCANTCPSKEKSLIMKPLETQLDQAKNWEYAMSLSYKENPLGTDTVKGSQFEKPLLEFSGACAGCGETPYARLVTQLFGDRMLIANATGCSSIWGGSAPSTPYTVNKDGHGPAWANSLFEDNAEFGFGMALAVKQQREKLADIVKEALELDLTQDLKNALKLWLDNFNSSEITKKTANIIVSLIQDYKTDDSKVKELLNEILDRKEYLVKKSQWIFGGDGWAYDIGFGGLDHVLASGEDVNVLVFDTEVYSNTGGQSSKATPVGAIAQFAAAGKGIGKKDLGRIAMSYGYVYVAQIAMGANQAQTIKALKEAESYPGPSLIIAYAPCINHGIKLGMGCSQIEEKKAVEAGYWHLYRYNPMLKAEGKNPFILDSKAPTASYKEFIMGEVRYSSLAKTFPERAEALFEKAEELAKEKYETYKKLAEQN

SEQ ID NO: 50 - protein, 311 aa, T. saccharolyticum
MSKVAIIGSGFVGATSAFTLALSGTVTDIVLVDLNKDKAIGDALDISHGIPLIQPVNVYAGDYKDVKGADVIVVTAGAAQKPGETRLDLVKKNTAIFKSMIPELLKYNDKAIYLIVTNPVDILTYVTYKISGLPWGRVFGSGTVLDSSRFRYLLSKHCNIDPRNVHGRIIGEHGDTEFAAWSITNISGISFNEYCSICGRVCNTNFRKEVEEEVVNAAYKIIDKKGATYYAVAVAVRRIVECILRDENSILTVSSPLNGQYGVKDVSLSLPSIVGRNGVARILDLPLSDEEVEKFRHSASVMADVIKQLDI

SEQ ID NO: 51 - protein, 741 aa, T. saccharolyticum
MINEWRGFQEGKWQKTIDVQDFIQKNYTLYEGDDSFLEGPTEKTIKLWNKVLELMKEELKKGVLDIDTKTVSSITSHDAGYIDKDLEEIVGLQTDKPLKRAIMPYGGIRMVKKACEAYGYKVDPKVEEIFTKYRKTHNDGVFDAYTPEIRAARHAGIITGLPDAYGRGRIIGDYRRVALYGIDRLIEEKEKEKLELDYDEFDEATIRLREELTEQIKALNEMKEMALKYGYDISKPAKNAKEAVQWTYFAFLAAIKEQNGAAMSLGRVSTFLDIYIERDLKEGTLTEKQAQELMDHFVMKLRMVRFLRTPDYNELFSGDPVWVTESIGGVGVDGRPLVTKNSFRILNTLYNLGPAPEPNLTVLWSKNLPENFKRFCAKVSIDTSSIQYENDDLMRPIYNDDYSIACCVSAMKTGEQMQFFGARANLAKALLYAINGGIDERYKTQVAPKFNPITSEYLDYDEVMAAYDNMLEWLAKVYVKAMNIIHYMHDKYAYERSLMALHDRDIVRTMAFGIAGLSVAADSLSAIKYAKVKAIRDENGIAIDYEVEGDFPKFGNDDDRVDSIAVDIVERFMNKLKKHKTYRNSIPTLSVLTITSNVVYGKKTGATPDGRKAGEPFAPGANPMHGRDTKGAIASMNSSKIPYDSSLDGISYTFTIVPNALGKDDEDKINNLVGLLDGYAFNAGHHININVLNRDMLLDAMEHPEKYPQLTIRVSGYAVNFNKLTREQQLEVISRTFHESM

SEQ ID NO: 52 - protein, 81 aa, T. saccharolyticum
MVITVCVGSSCHLKGSYDVINKLKEMIKNYGIEDKVELKADFCMGNCLRAVSVKIDGGACLSIKPNSVERFFKEHVLGELK

SEQ ID NO: 53 - protein, 572 aa, T. saccharolyticum
MSVINFKEANCRNCYKCIRYCPVKAIKVNDEQAEIIEYRCIACGRCLNICPQNAKTVRSDVERVQSFLNKGEKVAFTVAPSYPALVGHDGALNFLKALKSLGAEMIVETSVGAMLISKEYERYYNDLKYDNLITTSCPSVNYLVEKYYPDLIKCLVPVVSPMVAVGRAIKNIHGEGVKVVFIGPCLAKKAEMSDFSCEGAIDAVLTFEEVMNLFNTNKIGVECTKENLEDVDSESRFKLYPIEGKTMDCMDVDLNLRKFISVSSIENVKDILNDLRAGNLHGYWIEANACDGGCINGPAFGKLESGIAKRKEEVISYSRMKERFSGDFSGITDFSLDLSRKFIDLSDRWKMPSEMEIKEILSKIGKFSVEDELNCGACGYDTCREKAIAVFNGMAEPYMCLPYMRGRAETLSNIIISSTPNAIIAVNNEYEIQDMNRAFEKMFLVNSAMVKGEDLSLIFDISDFVEVIENKKSIFNKKVSFKNYGIIALESIYYLEEYKIAIGIFTDITKMEKQKESFSKLKRENYQLAQQVIDRQMKVAQEIASLLGETTAETKVILTKMKDMLLNQGDDE

SEQ ID NO: 54 - protein, 386 aa, T. saccharolyticum
MSHYIDIAHASLNKYDEELCGDSVQIIRKKDYAMAVMADGLGSGVKANILSTLTTRIVSKMLDMGSELRDVVETVAETLPICKERNIAYSTFTVVSIYGDNAHLVEYDNPSVFYFKNGVHKKVDRKCVEIGDKKIFESSFKLDLNDALIVVSDGVIHAGVGGILNLGWQWDNVKQYLSKVLEVYSDASDICSQLITTCNNLYKNRPGDDTTAIVIKVNESKKVTVMVGPPILKNMDEWVVKKLMKSEGLKVVCGGTAAKIVSRILNKDVITSTEYIDPDIPPYAHIDGIDLVTEGVLTLRKTVEIFKEYMNDKDSNLLRFSKKDAATRLFKILNYATDVNFLVGQAVNSAHQNPDFPSDLRIKVRIVEELISLLERLNKNVEVNYF

SEQ ID NO: 55 - protein, 504 aa, T. saccharolyticum
LFKFNTDVQMLKYEVLYNVAKLTLEDRLEDEYDEIPYEIIPGTKPRFRCCVYKERAIIEQRTKVAMGKNLKRTMKHAVDGEEPIIQVLDIACEECPIKRYRVTEACRGCITHRCTEVCPKGAITIINKKANIDYDKCIECGRCKDACPYNAISDNLRPCIRSCSAKAITMDEELKAAINYEKCTSCGACTLACPFGAITDKSYIVDIIRAIKSGKKVYALVAPAIASQFKDVTVGQIKSALKEFGFVDVIEVALGADFVAMEEAKEFSHKIKDIKVMTSSCCPAFVAHIKKSYPELSQNISTTVSPMTAISKYIKKHDPMAVTVFIGPCTAKKSEVMRDDVKGITDFAMTFEEMVAVLDAAKIDMKEQQDVEVDDATLFGRKFARSGGVLEAVVEAVKEIGADVEVNPVVCNGLDECNKTLKIMKAGKLPNNFIEGMACIGGCIGGAGVINNNVNQAKLAVNKFGDSSYHKSIKDRISQFDTDDVDFHVDSGEDESSETSFKEA

SEQ ID NO: 56 - protein, 581 aa, T. saccharolyticum
MDKVRITIDGIPAEVPANYTVLQAAKYAKIEIPTLCYLEEINEIGACRLCVVEIKGVRNLQASCVYPVSDGMEIYTNTPRVREARRSNLELILSAHDRSCLTCVRSGNCELQDLSRKSGIDEIRFMGENIKYQKDESSPSIVRDPNKCVLCRRCVATCNNVQNVFAIGMVNRGFKTIVAPSFGRGLNESPCISCGQCIEACPVGAIYEKDHTKIVYDALLDEKKYVVVQTAPAVRVALGEEFGMPYGSIVTGKMVSALKRLGFDKVFDTDFAADLTIIEEGNELLKRLNEGGKLPMITSCSPGWINYCERYYPEFIDNLSTCKSPHMMMGAIIKSYFAEKEGIDPKDIFVVSIMPCTAKKYEIDRPQMIVDGMKDVDAVLTTRELARMIKQSGIDFVNLPDSEYDNPLGESSGAGVIFGATGGVMEAALRTVADIVEGKDIENFEYEEVRGLEGIKEAKIDIGGKEIKIAVANGTGNAKKLLDKIKNGEAEYHFIEVMGCPGGCIMGGGQPIHNPNEKDLVRKSRLKAIYEADKDLPIRKSHKNPMITKLYEEFLISPLGEKSHHLLHTTYSKKDLYPMND

SEQ ID NO: 57 - protein, 282 aa, T. saccharolyticum
LNDILVKARNNKYAIGGFNFNFYDDALGIISAAYELKSPIILMASEGCVKFLGVKHIVNFVNQLKDEYNIPIILHLDHGKDIEIIKNCIDNKFDSIMYDGSLLNFEENIKNTKFIADLCHDKGMTIEGELGRISGAEENIENSEDVFTDPDSVAEFTERSDVDSLAVAIGNAHGLYKGRPRLDFERLSKINKISKVPLVLHGGTGIPYEDIQKAIQLGISKVNVGTEIKIAYIKSIKKHLETINDNDIRHLVSMVQNDIKELVKQYLDIFGTANKYSQLQSM

SEQ ID NO: 58 - protein, 283 aa, T. saccharolyticum
MLVTGIELLKKANEEGYAVGAFNTSNLEITQAIVEAAEEMRSPAIIQVSEGGLKYAGIETISAIVRTLATKASVPIALHLDHGTDFNNVMKCLRNGWTSVMMDASKLPLEKNIEVTKNVVTIAHGMGVSVEAEIGKIGGTEDNVTVDEREASMTDPDEAFKFAKETGVDYLAISIGTAHGPYKGEPKLDFDRLVKIKEMLKMPIVLHGASGVPEADIRKAVSLGVNKINIDTDIRQAFAARLRELLKNDEEVYDPRKILGPCKEAMKEVIKNKMRMFGSEGRA

SEQ ID NO: 59 - protein, 400 aa, T. saccharolyticum
MITGDQLLIKQINKSIVLNTIRKKGLISRADLANITGLNKSTVSSLVDELIKEGFVEEEGPGESKGGRKPIMLMINSLAGCVIGVDLDVNYILVILTDILANILWQKRINLKLGESKEDIISKMLELIDEAIKNSPNTVKGILGIGIGVPGITDYKRGVVLKAPNLNWENVELKKMVEERFNLKVYIDNEANTGAIGEKWFGGGRNAKNFVYVSAGIGIGTGIIINNELYRGSNGLAGEMGHMTIDINDHMCSCGNRGCWENYASEKSLFRYIKERLEAGQEDDFIDSENIDSLDINDIAGYAELGSKLAIDAINEISKNLSVGIVNIVNTFNPDLVLIGNTLSAIGDMLIDAVKEYVREKCLVSRYNDIAIEISKLGMLERAIGAVTLVISEVFSYPGL

SEQ ID NO: 60 - protein, 451 aa, T. saccharolyticum
MTNVLNFDYSNALNFVNEHEISYLEKQALLSLDMVLNKTAQGSDFLGWVDLPKDYDKEEFARIKKAAEKIKSDSDALVVIGIGGSYLGARAAIEMLTHSFYNVLPQSVRKAPEIYFAGNSISSTYLQDLLEILEGKDVSINVISKSGTTTEPAIAFRVFRDFLEKKYGKEEAKSRIYVTTDRQKGALKKLADEEGYETFVIPDDVGGRYSVLTAVGLLPIAAAGIDIDEMMKGAYDASIVFKKPDIKENLSMQYAVLRNALYRKGKSVEILVNYEPRLHYFSEWWKQLYGESEGKDHKGIYPASVDFSTDLHSMGQFIQDGSRIMFETVINVEKPLKEITINEDKDNVDGLNFLTGKTVDLVNKKAFEGTVLAHNDGGVPNLIVNVPEISAYNFGYLVYFFEMACGISGYLNGVNPFDQPGVEAYKKNMFALLGKPGYEKEKEELEKRLKR

SEQ ID NO: 61 - protein, 372 aa, T. saccharolyticum
MYNIQLDSPNLGDKEKDYLVKCIESGYVSTVGPFVPEFERRFAEFLNVNHCVSVQSGTAALYMALYELGIKDGDEVIVPAITFVATVNPIVYCGATPVFVDVDKDTWNIDPKEIEKAITPKTKAIIPVHLYGNPCDMDKIMEIAKENNIYVIEDATESLGALYKGRMTGTIGHIGCFSFNGNKVITTGGGGMVASNNEDWVSHIRFLVNQARDMTQGYFHTEIGFNYRMTNLEASLGIAQLERLAGFLEKKRMYFEIYKKIFNGIEEISLQTEYEGAKSSDWLSSVKIDCKKVGMTIHQIQDELKRRGIPTRRIFNPIVDLPPYKKYKKGSYSNSYEIYENGLNLPSSTLNTYEDVKYVAKTLLDILSIKKR

SEQ ID NO: 62 - protein, 253 aa, T. saccharolyticum
MLAIERRKRIMRLIQENQSVLVPELSKLFNVTEETIRRDLEKLEAEGLLKRTYGGAVINENSSADIPLNIREITNIESKQAISMKVAEYIEDGDTLLLDSSSTVLQVAKQLKFKKKLTVITNSEKIILELANAKDCKVISTGGVLKQNSMSLIGNFAEDMIKNFCVDKAIISSKGFDMTNGITESNEMEAEIKKAMANSAEKVFLLLDHNKFDKSSFVKMFDLDKIDYLFTDRKLSLEWEEFLKKHNIDLIYC

SEQ ID NO: 63 - DNA, 761 bp, T. saccharolyticum
atgcttgcgatagaacgaaggaagaggataatgaggcttatacaggaaaatcaaagcgtttggtgcctgagttaagtaaattgtttaatgtgacagaggaaactataaggagagatttagagaaacttgaagcagaagggcttttaaagaggacttatggtggtgctgttataaatgaaaattcaagtgctgatatccccttaaatataagggaaataacgaatatagaaagcaaacaggccataagtatgaaggttgccgaatacattgaagatggtgatacacttttgcttgattcaagctctacagttcttcaagtagcaaagcaattaaaattcaaaaagaagcttacagtcataacaaattcggaaaagataatattagaattagcaaatgcgaaagattgcaaagtcatttctacaggaggagtattgaagcaaaattctatgtcgctaattggaaatttcgcggaagatatgataaaaaatttctgtgtagataaagccataatatcatcaaaaggttttgacatgacaaatggcattacagagtcaaacgaaatggaagctgaaataaaaaaagccatggccaactcggcagaaaaagtgtttttacttcttgatcacaacaaatttgacaagtcatcgttcgtcaagatgtttgacttagataaaatcgattatctatttaccgatagaaagctgtctttagaatgggaagaattcttgaaaaaacacaatattgatttaatctattgttag

SEQ ID NO: 64 - protein, 259 aa, T. saccharolyticum
VYSEYEVKKQICEIGKRIYMNGFVAANDGNITVRIGENEIITTPTGVSKGFMTPDMLLNINLNGEVLKSSGDYKPSTEIKMHLRVYRERPDVKSVIHAHPPFGTGFAIVGIPLTKPIMPEAVISLGCVPIAEYGTPSTEELPDAVSKYLQNYDALLLENHGALTYGPDLISAYYKMESLEFYAKLTFISTLLGGPKELSDSQVEKLYEIRRKFGLKGRHPGDLCSTLGCSTNSAKSNDDDISELVNVITKKVLEQLKYN

SEQ ID NO: 65 - DNA, 780 bp, T. saccharolyticum
gtgtattctgaatatgaggtaaaaaaacagatctgcgaaataggaaagagaatctacatgaatgggtttgtggcagcgaatgacggcaatatcaccgttaggattggtgaaaatgaaataataacgacgcctaccggtgtcagcaaaggtttcatgactccagacatgctattaaatattaatttaaacggtgaagtattaaaatcttcaggcgactacaaaccgtccacagaaataaagatgcatcttagagtctatagagaaaggccagatgtcaaatcagtcatacatgcacatccaccatttggcacaggttttgctattgtagggatcccgcttacaaagccaataatgccagaagcagttatatctttaggctgtgtgccgatagccgaatacgggacgccttctacagaagagctgccagatgccgtctctaaatatttgcaaaattacgatgcgcttttattagaaaatcatggtgcgttgacatacggtcctgatttaattagcgcatactacaagatggaatcacttgaattttacgcaaaattgacatttatttctacacttctcggaggtccaaaagaattatcagatagccaagtagaaaagctttatgaaattaggagaaaattcggtttaaaaggaagacatccaggcgatttgtgcagtacattaggatgcagcacaaattctgcaaaatcgaatgatgatgacatttctgaacttgtgaatgttatcactaagaaagtattagaacaattgaaatacaattaa

SEQ ID NO: 66 - protein, 554 aa, T. saccharolyticum
MKHSKRFEVLGKRPVNQDGFINEWPEKGFIAMCSPNDPKPSIKIENDKIVEMDGKRREDFDFIDLFIADHAINIYQAEKSMKMNSLDIAKMLVDINVERKTIIKVVSGLTPAKIMEVVNHLNVVEMMMAMQKMRARKIPANQSHITNLKDNPVQIAADAAECALRGFREEETTVGVTKYAPFNAIALLIGSQALKRGVLTQCAVEEATELELGMRGFTTYAETISVYGTESVFIDGDDTPYSKAFLASAYASRGLKMRFTSGTGSEVLMGNAEGKSMLYLEIRCIMVTKGAGVQGLQNGAISCIGITSSVPSGIRAVLAENLIASMLDLEVASGNDQTFTHSDIRRTARTMMQFLPGTDFIFSGYSGTPNYDNMFAGSNFDAEDFDDYNVLQRDLMVDGGLRPVKEEDVVEVRRKAAKALQDVFRELNLGVVTDEEVEAAAYAHGSKDMPERDVLSDLESIDEMMKRGITGIDIVKALYRSGHEDIAENILNMLKQRISGDYLQTSAILDEDFNVISAINCPNDYLGPGTGYRIDKDRWEEIKNIPYTINPDNL

SEQ ID NO: 67 - DNA, 1665 bp, T. saccharolyticum
atgaaacattctaagcgatttgaggttctcggcaaaagacctgtaaatcaggatggatttataaatgaatggccagaaaaaggcttcatagcaatgtgtagtcccaatgatcctaagccatcaataaagattgaaaacgacaagatcgttgagatggatgggaagagaagagaagactttgattttatagatttattcatagctgatcacgctataaatatttatcaggctgagaaatccatgaaaatgaactcgcttgatatagccaaaatgcttgtagatataaatgtagagagaaagactataataaaagtagtttcgggacttacacctgccaaaataatggaagttgtaaatcatcttaatgtcgttgaaatgatgatggctatgcagaaaatgcgagcaagaaagattccggctaatcaatcacatattacaaatcttaaagataatcctgtgcagattgcagcggatgctgccgaatgtgctttaagaggttttagggaagaagagaccaccgtaggagtgacaaaatatgctccgtttaatgcaatagcgttattgatagggtctcaggcattaaaaagaggcgtgcttactcaatgtgctgttgaggaggcgacggaacttgaattaggcatgaggggatttaccacatacgctgagactatatctgtttatggaactgaaagtgtttttatagatggtgacgatacaccttactccaaagcattccttgcttctgcttatgcgtcaagaggattgaaaatgaggtttacgtcaggtacaggttcagaagttcttatgggaaatgcagagggtaaatcgatgttgtacctggaaatcaggtgcatcatggttacaaaaggtgcaggagtgcaggggcttcaaaatggtgcaataagctgtataggcataactagctcagttccttcaggtataagggcggtgctggctgaaaaccttatagcatctatgcttgatttagaggtagcatcaggcaatgatcagacttttacacattcagacataagaaggacagcaaggactatgatgcagtttttacccggtactgatttcatattttcaggttacagtggaacgcctaattatgacaatatgtttgcaggttccaattttgatgcagaagattttgatgactacaatgtactgcaaagggatttaatggtagatggagggttaaggcctgtaaaagaagaagatgtggtagaagtgaggcgaaaggcagctaaagctttgcaggatgtatttagagagttaaatcttggagtagttacagatgaagaagtagaagcagcagcatatgcacacggcagcaaagatatgcctgaaagagatgttttgtctgaccttgaatcaatcgatgagatgatgaaaagagggattacaggcattgacatcgtaaaggctttatatagatctggacatgaggatatagcggaaaacattttaaacatgttaaaacagcgcatatctggagactatttgcagacatcagctattcttgatgaagattttaatgttataagcgccataaattgtccaaatgattacttaggacctggaacaggatataggattgataaagatagatgggaagagataaagaatattccttacaccattaatcctgacaatttgtaa

SEQ ID NO: 68 - protein, 208 aa, T. saccharolyticum
MYVDEELLKEITKRVIEELNNKHKTDNVPSYFIENGVAYKGKNIEEVVIGVGPAFGKHIKKTINGLDHRDVIKEIIAGIEEEGMVHRIVRVLKTSDVAFIGKEAALLSGSGIGIGIQSKGTTVIHQKDLYPLSNLELFPQAPLLNLELYREIGKNAARYAKGMMVKPILIQNDYMVRPKYQVKAAIMHIKETEKILKNAQSIQLTIDL

SEQ ID NO: 69 - DNA, 627 bp, T. saccharolyticum
atgtacgtagatgaagaactgttaaaagaaattactaaacgtgttatagaagaattaaataataagcataaaactgataatgtgccttcgtattttattgaaaatggagttgcctataagggtaaaaatatagaggaagtcgtcattggtgttgggcctgcatttggaaagcatataaaaaagactataaatggccttgaccatagagatgtcataaaagaaataattgcaggcatcgaagaagaaggtatggttcatagaattgtaagagttctaaagacttctgatgtggcgttcataggcaaagaagctgctttattaagcggatcgggaataggcataggcatacaatcaaaaggtactacagtgattcatcaaaaagatttatatcctttaagcaatttagaactgtttccacaagctccactgctaaatttagaattatacagggaaataggcaaaaatgcggcgagatatgctaaaggcatgatggtaaagcctattttgattcaaaatgattacatggtgagacctaaataccaagtgaaagctgctataatgcatataaaagagacggaaaagatattgaaaaatgctcaatcaatccaattgacgatagacttgtaa

SEQ ID NO: 70 - protein, 132 aa, T. saccharolyticum
MEEYPLSKSAFDKLVTKTGKHLNEINIENVMKGNVKPDDIKISKEVLLMQGQIAERYGRHQMKENFTRASELTDVPDEKILEIYESLRPFRSTKEELINLAYELRDKYNAINCANLILEAAEVYEKRNILKT

SEQ ID NO: 71 - DNA, 399 bp, T. saccharolyticum
atggaagaatatccgctatcaaaaagtgcttttgataaattggtgacaaaaacaggcaaacatttgaatgaaataaatattgaaaatgtaatgaagggaaacgtaaaacccgatgatatcaagatatccaaagaagtgcttttaatgcaagggcaaattgcagaaagatacggcaggcatcagatgaaggagaatttcacaagagcatcggagcttacagatgttccagatgaaaagattttggaaatatatgagagcttaaggccgtttagatctacaaaggaagagcttataaatcttgcctatgaattaagagataagtacaatgccattaactgtgcaaacttgatacttgaggctgctgaagtatatgaaaaaagaaatattttgaaaacttaa

SEQ ID NO: 72 - protein, 605 aa, T. saccharolyticum
MKLIAGVDIGNSTTEVCIAAIKDDNTLEFLSSSLTATTGVKGTVDNVTGVINGLTEALKKIGKNIRDLSLIRINEAAPVVCGAAMETITETVITGSTMIGHNPSTPGGVGLGVGEIIHINDLADATKGKNYIVVIPKEIGYEEASIMINKSFENDIDVKAAIVQSDEAVLINNRLKKIIPIVDEVRQIEKIPSGVVAAVEVAPEGKSISTLSNPYGIATIFDLTPEETKYVIPISKSLMGKKSAVVIKTPRGQVKERIIPAGNLLIMGPTMSSKVSVDSGAEAIMESVEEVGTIDDVEGEENTNVGNMIKNLKNKMANITGQKVDKIKIKDIFAVDTTVPVKVEGGLAGETSMEKAVVLAAMVKTDTLPMIEIAEKLQRKLGVFVKIAGVEAVMATLGALTTPGTKLPLAILDIGGGSTDAALIDEKGIVKSIHMAGAGELVTMLIDSELGLNDRYLSEEIKRNPIGKVESLFHIRMENREIKFFDKPLNPRYYGRIVILKENDMIPVFKEDLTMEKIIYVRRQAKDKVFVKNAIRALKKIAPENNLRRIPNVVLVGGSALDFEIPEMILSELSKYKIIAGRGNIRKIEGPRNAVATGLVMSYLG

SEQ ID NO: 73 - DNA, 1817 bp, T. saccharolyticum
atgaaactcatagcaggtgttgatattggcaattctacaacagaagtgtgtatagccgctattaaagatgacaatacattagaatttttaagcagttccttgacagctacgacaggtgtaaaaggcactgtggataatgtgacaggggttattaatggattgactgaggcactaaaaaaaattggcaagaatattagggatttaagcctcattagaatcaatgaagccgccccagttgtctgtggtgctgctatggagacaataacggaaactgttatcactggttcgactatgataggtcataatccatccacgccgggtggtgtcggacttggagtaggcgagataatacatataaatgatttagctgatgctactaaaggcaaaaattacattgtggttatacctaaggagattggctatgaagaagcttcaataatgataaacaaatcttttgaaaacgatattgatgtaaaagctgctatagttcaaagcgatgaagcagttttaatcaacaacaggcttaaaaagattataccaattgttgacgaagtaaggcagatagaaaagattccatcgggtgttgtagcggctgtagaggtggcaccagaaggcaagtccataagcacgttatcaaatccttatggtatcgcaacaatatttgacttaactccagaagagacaaagtatgtcataccgatttcgaaaagtttgatggggaaaaagtcagcagttgtcataaaaacaccgaggggacaagtgaaagaaagaataattccggctggtaatctcttaatcatggggcctactatgtcatcaaaagtaagtgttgattctggtgctgaagctataatggaatcagttgaagaagtcggcacaattgatgacgtagaaggtgaagaaaatacaaatgttgggaatatgataaaaaatctaaaaaacaagatggcaaatataactgggcaaaaagtagataagataaagattaaagatatcttcgctgttgatacgacagtccctgttaaagtagagggcggacttgctggtgagacttcaatggaaaaagcagtcgtgttggcggctatggtaaagacagatacgcttcgatgatagaaattgcagaaaagcttcaaagaaagttgggtgtatttgtaaaaatagctggagtagaagctgtgatggctacattaggtgcgcttacaactccaggcacaaagttgccacttgcaatactggatatcggtgggggttctacagatgcagctttgattgatgaaaaaggcattgtaaaatctatacacatggcaggtgctggagaattagtcacaatgcttattgattcagaattagggttaaatgatagatatttgtctgaagaaataaagagaaatccgattggaaaagttgaaagcctatttcacataagaatggaaaatagggagataaagttttttgacaaacctttaaatcctcgatattacggtaggatcgtaattttaaaagaaaatgacatgatccctgtatttaaagaagatttgacaatggaaaagattatttacgtgcgaagacaagcgaaggataaagttttcgttaaaaatgctattagagctttgaaaaaaattgctccggaaaataatttaaggcgaataccaaatgtagtcttggttggcggttctgctttggactttgaaattccagagatgattttatcagagctatcaaaatacaaaatcatagcaggcagagggaatataagaaaaatcgaagggccaagaaatgctgtagcgacaggtcttgtgatgtcttatttagggtga

SEQ ID NO: 74 - protein, 112 aa, T. saccharolyticum
MEFIKPQIVIFANTENKYIINEVIAGIEEEGALYRLSYNECADVMKMAYDAAKASVLGIGIGISGDLVCLHSKNLEINTPLILSKTSENFDPRLVGCNAAKYVKGLPLKYLD

SEQ ID NO: 75 - DNA, 339 bp, T. saccharolyticum
atggaatttataaagcctcaaatagtgatttttgcaaatacagaaaacaaatatataataaacgaggttatagctggcattgaagaagaaggtgcattatatagattatcttacaatgaatgtgctgatgttatgaaaatggcttatgatgcagcaaaagcatctgtattaggtatcggaataggcatatctggagatttagtgtgtttgcactctaaaaacttggaaatcaatacacctttgattctttcaaagacaagtgaaaactttgatccacgactcgttggatgcaatgctgcaaaatatgtaaagggtttgccacttaaatacttagattag

SEQ ID NO: 76 - protein, 56 aa, T. saccharolyticum
MSVYTKTGDDGYTLLLNGERIPKDDLRIETLGNLDELTSYLGFAKAQINDDSIKKR

SEQ ID NO: 77 - DNA, 171 bp, T. saccharolyticum
atgagtgtttatactaaaactggtgatgatggttacacgttgctattaaatggagaaagaattccaaaggacgatttgagaatagagacattgggaaatttggatgaattgacaagctatttaggatttgcaaaagctcaaataaatgatgattccataaaaaagagatag

SEQ ID NO: 78 - protein, 225 aa, T. saccharolyticum
MVKIKNGFVIPGKNQISALLDIVRTITRKTERSLIKVDKKYPVNINSKVYINRLSDYLFVLARYMEIRTEIEEKVKDVIRKHYGKNKGEIKLNLDIAKNLMAKVEKKAESINLPVAIAIVDMHGNLIAAHFMDGTLLESMNLAINKAYTSVVLKMSTQELSKLAQPGQPLYGINTTDNRIVVFGGGCPIKHQGEIVGGIGVSGGTVEQDIELSIYGADVFEEVIS

SEQ ID NO: 79 - DNA, 678 bp, T. saccharolyticum
atggtaaagattaaaaatggttttgtaatacctggtaaaaaccaaatctcagcattattagatattgtaaggactataacgagaaaaactgagagaagcttaatcaaagttgacaagaaatatcctgtaaatattaattcgaaagtttacatcaatagattgtctgattatttgtttgttttagcaaggtatatggaaataagaacggaaatagaagaaaaagtaaaagacgtgataagaaagcattatggaaagaacaaaggcgaaataaagctaaatttagatatagcaaaaaatttaatggctaaggtagaaaagaaggcagaaagcattaatctaccggttgctattgcaatagttgacatgcatggcaatttgatagcggctcattttatggatggtacacttcttgaaagcatgaatctagctataaataaagcttatacatcagtggtgcttaaaatgtcgacgcaagagttatcaaaacttgcacaaccagggcagcctctttacgggataaatacaactgataatagaatcgtagtgtttggaggtgggtgccctataaaacatcaaggtgaaatagttggtggaattggagttagcggtggtacagtagaacaagatatagaactttctatttatggtgcagatgtatttgaggaggttatatcatga

SEQ ID NO: 80 - protein, 467 aa, T. saccharolyticum
MKVKEEDIEAIVKKVLSEFNFEKNTKSFRDFGVFQDMNDAIRAAKDAQKKLRNMSMESREKIIQNIRKKIMENKKILAEMGVSETGMGKVEHKIIKHELVALKTPGTEDIVTTAWSGDKGLTLVEMGPFGVIGTITPSTNPSETVLCNSIGMIAAGNSVVFNPHPGAVNVSNYAVKLVNEAVMEAGGPENLVASVEKPTLETGNIMFKSPDVSLLVATGGPGVVTSVLSSGKRAIGAGAGNPPVVVDETADIKKAAKDIVDGATFDNNLPCIAEKEVVSVDKITDELIYYMQQNGCYKIEGREIEKLIELVLDHKGGKITLNRKWVGKDAHLILKAIGIDADESVRCIIFEAEKDNPLVVEELMMPILGIVRAKNVDEAIMIATELEHGNRHSAHMHSKNVDNLTKFGKIIDTAIFVKNAPSYAALGYGGEGYCTFTIASRTGEGLTSARTFTKSRRCVLADGLSIR

SEQ ID NO: 81 - DNA, 1404 bp, T. saccharolyticum
atgaaagttaaagaggaagatattgaagcgatcgtcaaaaaagtcttatcggaatttaattttgaaaaaaatactaaaagtttcagagattttggcgtatttcaagatatgaatgatgctattcgtgctgcaaaagatgcccagaaaaaattgagaaatatgtccatggagtcgagagaaaagattatacagaatataagaaaaaagattatggagaataaaaaaatacttgcagagatgggcgtcagtgaaactggcatggggaaagtagagcacaaaataataaaacatgagcttgtagcacttaagacacctggtaccgaagatatagtgacaacagcatggtctggcgataagggactgacattggttgaaatggggccatttggtgtaataggtacgattactccttcgacaaatccaagtgaaaccgtcctttgcaatagcataggtatgatagccgcaggtaattcagtcgtatttaatccacatccaggtgcggtaaatgtatctaattacgctgtcaagttagtaaatgaagcggtgatggaagctggcggccctgagaatttagtcgcatctgttgaaaaacctacacttgaaactggaaatattatgttcaagagtcctgatgtttcgctattagtagcgacaggcggacctggtgtagtaacatcggttctctcatctggcaaaagggcaataggagcaggagcaggaaatccaccagttgtagttgatgaaacggcagatataaaaaaagctgcgaaagatatagtcgatggtgctacatttgacaacaatttgccttgtattgctgaaaaggaagtagtttctgtagataaaataacagatgaactgatttactacatgcaacagaatggctgctacaagattgaggggcgagaaattgaaaagctcattgaacttgtattggatcacaaaggtggcaagataacattaaacaggaaatgggttggcaaagatgctcatttaatactaaaagctataggcatagatgctgatgaaagcgtaaggtgcataatttttgaggcggaaaaagacaatccgttagtggtagaagagctgatgatgcctattttaggaatagtaagagccaaaaatgtagatgaagcgataatgattgcgacagagttagaacatggcaataggcattcagcacatatgcattctaaaaacgttgataatttaacaaagtttggaaaaataattgacactgctatatttgtaaaaaatgctccatcgtatgccgcgttaggatatggtggtgaaggttattgcacatttacgattgcaagcagaacaggtgaaggattgacatctgcaaggacttttactaaaagtcgtagatgtgtcttggcagatggattatcaataagatag

SEQ ID NO: 82 - protein, 403 aa, T. saccharolyticum
MEVNQIDIEEIVKKILNDLRNEPKENIKESNSKIPSICRAAVLTDVKKIEVKEFNIPEINDDEMLVKVEGCGVCGTDVHEYKGDPFGLIPLVLGHEGTGEIVKLGKNVRRDSAGKEIKEGDKIVTSVVPCGECDICLNHPDKTNLCENSKIYGLISDDNYHLNGWFSEYIVIRKGSTFYKVNDINLNLRLLVEPAAVVVHAVERAKSTGLMKFNSKVLVQGCGPIGLLLLSVVKTLGVENIIAVDGDENRLNMAKRLGATALINFTKYSNIDELVDAVKKASDGIGADFAFQCTGVPSAASNIWKFVRRGGGLCEVGFFVNNGDCKINPHYDICNKEITAVGSWTYTPQDYLTTFDFLKRAKEIGLPIEELITHRFSLDKMNEAMEVNMKQEGIKVVYINDRF

SEQ ID NO: 83 - DNA, 1212 bp, T. saccharolyticum
atggaagtcaatcagatagacattgaggagatagttaagaaaatattaaatgatttaagaaatgagcctaaagaaaacattaaagagagcaattcaaaaataccatctatctgcagagctgctgtacttacagatgttaaaaaaatagaagtaaaagaatttaatattccagaaataaatgatgatgaaatgcttgtcaaggtggaaggctgtggcgtttgcggtactgatgttcatgaatacaaaggagatccttttggacttataccattggttttaggacacgaaggtacaggtgagatagtcaagctggggaaaaacgtgagacgagattctgctggtaaagaaatcaaagaaggcgataagattgttacatctgtcgttccgtgcggtgaatgcgatatatgtttgaatcatccagacaagacaaatttgtgtgaaaactcaaagatttacggcttaatatccgatgataattaccatttaaatggttggttctcagagtacatcgtcataaggaaaggctcaacattttataaggtcaatgatataaaccttaatttgaggcttttggtagaaccggctgcagtagtcgtacatgcagtagagcgcgcaaaatccacaggtcttatgaaattcaacagtaaagttctcgtacaaggctgtggccctataggattactgctattgtcggttgtaaagacgcttggagtagaaaatatcatagccgtcgacggcgatgagaatagactcaacatggctaaaagattaggtgctacagcactcattaattttactaaatacagcaatattgatgagcttgttgatgctgttaaaaaagcaagcgatggaattggcgcagattttgcatttcaatgtacaggcgttccttctgcagcgtctaatatttggaagtttgtaaggcggggaggtggtttatgcgaagttggattttttgtaaataatggtgattgtaagataaacccccattatgatatttgcaataaggagataacagcagttggctcatggacttacactcctcaagactatttgacaacttttgattttctcaaaagagctaaagaaataggacttccaattgaagagctgataacacatagattttcacttgataaaatgaatgaagctatggaagttaatatgaagcaggaagggataaaagtagtgtatataaatgacagattttag

SEQ ID NO: 84 - protein, 101 aa, T. saccharolyticum
MQAVGLIEVYGLVAAFVAADAACKKANVVIESFDNNKPLNAEALPVPLIIVVKLRGDLEDVKIAVDAAVDAANKISGVVATNIIAKPEEDTEKLLKLNCLK

SEQ ID NO: 85 - DNA, 306 bp, T. saccharolyticum
atgcaggctgttggattgattgaagtttatggattagtagcggcatttgtggcagcagatgctgcatgcaaaaaagcgaatgtcgtaatagagtcttttgacaacaataagccattaaatgctgaagcattgccagttccattgataatagtcgttaagctcagaggagatcttgaggatgtaaaaatagcggtagatgctgcagttgatgcagctaataaaatatctggtgtagttgctacaaatataatagcaaaaccagaagaagatactgaaaagctattaaagctaaattgtcttaaataa

SEQ ID NO: 86 - protein, 92 aa, T. saccharolyticum
MVQEALGMVETRGLVAAIEAADAMVKAADVTLIGTEKIGSGLVTVMVRGDVGAVKAATEVGASAASKLGELVAVHVIPRPHTDVEKILPTIK

SEQ ID NO: 87 - DNA, 279 bp, T. saccharolyticum
atggtacaagaagcattgggaatggtagaaacgagaggattggtagcagcaatagaagcagcagatgctatggtaaaggctgcggatgtcactttgataggaactgaaaaaataggttcaggacttgtaacagtcatggtaagaggagatgtcggtgcagtaaaagcagcgacagaagttggcgcaagtgcagcttcaaaattgggagagttagtggctgttcacgtaataccaaggcctcatactgatgttgaaaagatactgccgacaattaaataa

SEQ ID NO: 88 - protein, 105 aa, T. saccharolyticum
MYAIGLIEVNGFVTAVETLDAMLKTANVEFVTWEKKLGGRLVTIIIKGDVSAVEEAILTGKIEADKITRTVAYAVIPNPHPETIKMVNISAGKLFKADGGEINEF

SEQ ID NO: 89 - DNA, 318 bp, T. saccharolyticum
atgtatgcaattggacttattgaagtaaatgggtttgtcacagcggttgaaacactggatgcaatgttgaaaacagccaatgtagagtttgtaacatgggagaaaaaacttggaggcagacttgtgacaatcattattaaaggagatgtttcagcagttgaagaagcaattttaactggaaagattgaagctgacaagattacacggacagtagcatacgcagttattccaaatccacatccagaaactataaagatggtaaatattagtgcaggaaagctatttaaagcagatggtggtgaaataaatgagttctga

SEQ ID NO: 90 - protein, 127 aa, T. saccharolyticum
MSSEEKDTNAKDVKVEKQKNNLTKTSNKEFKEELIMEQQALGMVETRGLVAAIEAADAMVKAANVTLIGTEKIGSGLVTVMVRGDVGAVKAATETGANAAKKLGELVAVHVIPRPHADVEKILPTIK

SEQ ID NO: 91 - DNA, 384 bp, T. saccharolyticum
atgagttctgaagaaaaggatacgaatgcaaaagatgttaaagtcgaaaagcagaaaaataatttaacgaaaacatcaaataaagaatttaaggaggaattgattatggaacaacaagcattaggaatggtagagacgagaggattggtagcagcgatagaagctgctgatgcaatggtaaaggctgctaatgtcacgttaataggaactgaaaaaataggttcaggacttgtaacagtcatggtaagaggagatgttggtgcagtaaaagcagcgacagagactggagcaaatgcagctaaaaagttaggggagttagtagctgttcacgtaataccaagacctcatgcagatgtagagaaaatactgcctacgataaagtag

SEQ ID NO: 92 - protein, 217 aa, T. saccharolyticum
VITVNEKLIEIISKTIADTISERNSLKIPVGVSARHVHLTKEHLDILFGKDYILKKKKELMGGQFAAEECVTIIGFKLNAIEKVRVLGPLRDKTQVEISKTDAISLGLNPPIRESGDIKGSSPITIVGPRGAISLKEGCIIAKRHIHMSPEDSKRFNVKDDDIISVKINGQRGGILENVQIRVDEKYTLEMHIDTDEANCMGLKSGDFVEIVRDNRS

SEQ ID NO: 93 - DNA, 654 bp, T. saccharolyticum
gtgataacagtgaacgaaaaattgatagagattatatcaaaaactatagcggatacgattagtgaaaggaattcgcttaagataccagtaggcgtatcagcccgacatgtacatctgactaaagaacatttggatatattatttggaaaagattatatccttaaaaagaaaaaggaattgatgggtggacagttcgcagcagaggaatgtgtgacaattatcggatttaaattaaatgctattgagaaagtgagagttttgggtcctttaagagataaaacgcaggtagaaatatcgaagaccgatgcaataagtttagggttaaaccctcctatacgggaatcaggtgatataaaaggttcatcgccaattacaattgtagggccgagaggagcaatatcattaaaagaaggatgtataatagcaaaacgacatattcacatgtcaccggaagattccaaaagattcaatgttaaagacgacgatataatatcagtaaaaataaatggtcagcgaggcggaattttagaaaatgtacagattagagttgacgaaaagtatacacttgagatgcatattgacacagatgaagctaattgcatgggactaaaaagcggcgattttgttgaaatagtaagagataataggagttga

SEQ ID NO: 94 - protein, 92 aa, T. saccharolyticum
LIIAKVVGTVISTRKNQNLIGNKFLIVEPVSEMNYDSKNRVVAIDNVGAGVGEIVLVTFGSSARIGCGMPDSPVDAAIVGIVDSIKDIIIDD

SEQ ID NO: 95 - DNA, 279 bp, T. saccharolyticum
ttgataatagctaaagttgttggtactgttatttctacccgcaagaatcaaaatttaataggcaataaatttttaatagtagaaccagtaagtgaaatgaattatgacagtaaaaatagggttgttgcaatagataatgtaggtgcaggtgtaggagagatagtattagttacctttggaagttcagcaagaatcggttgtggtatgccagattcgcctgtagatgcggcaattgtcggaattgttgatagcataaaagatattatcattgatgattag

SEQ ID NO: 96 - protein, 456 aa, T. saccharolyticum
MMNIDELKNIVFENGIVGAGGAGFPTHAKLTTGIDTIILNGAECEPLLRVDRQLLAIYTDEILMTLSFIVDTLGAKRGIVAIKSAYKTAISSVKNLIGNYKNLELKVLPDVYPAGDEVVLIYETTGRIVPEGSIPISVGTLVMNVETVLNVYNAIYLKHPVTEKYVTVTGDVKYPSTFKAKVGTSVARLIEKAGGCLEKDCEVIMGGPMTGKIVDVKTPITKTTKAIIVLPKDHPVITKRKTNIRIGLKRAMSVCSQCQMCTDLCPRNLLGHSIKPHKVMNAVANSIIDDTAAYTMTMLCSECGLCEMYSCHQSLSPRKIISQIKIKLRQNGVKNPHNKRPETANVMRDERLVPMERLISRLSLKKYDVDAPMNFDTVIPSHHVVMQLSQHVGAKAIPVVKVGDIVKEGDLIGDVPNNKLGAKLHASIDGIIIDVTDDSIVIKPRGDFDGQSDRIG

SEQ ID NO: 97 - DNA, 1371 bp, T. saccharolyticum
atgatgaatattgatgaacttaaaaatatcgtatttgaaaatggaatagtcggtgcaggcggagctggatttcctacacatgcaaaacttactacaggtatagatacaatcatattaaatggcgctgaatgtgaaccgcttttaagagtagataggcagctacttgcaatatatactgatgaaatattgatgactttatcattcatagttgatactttaggagccaaacgtggcattgtagcaataaaatcagcatacaaaactgccatcagctcagttaagaatttgattggtaattataaaaacttggagttaaaggtattgccagacgtttatcctgctggtgatgaagttgtattaatatatgaaacgactggaagaattgtgccagaaggttctatacctatttctgttggcacgttggtaatgaatgtggaaactgtgcttaatgtttataatgctatttatttaaaacatccagtcacagaaaagtatgtaacagtaacgggagatgtcaaatatcccagcacatttaaagcaaaagtaggaacatctgtagctcgtcttattgaaaaagcaggaggatgcttagaaaaagattgtgaagtgataatgggtggtcctatgactgggaaaatagttgatgtaaagactccaataacaaaaactacaaaagctattatcgttctcccaaaagaccaccctgtgataacaaagagaaagacaaacataaggatagggttaaaacgagcaatgtctgtttgctctcaatgccaaatgtgcacagatctatgtcctagaaatttattaggtcattccatcaaacctcataaagtcatgaatgcagttgcaaatagtattattgatgataccgctgcatatacgatgacaatgttatgttctgaatgtggattgtgcgagatgtattcatgtcatcaaagtttgtcgccgagaaagataataagccagataaagataaaattaaggcaaaatggtgtaaaaaatccacacaacaaaagaccagaaacagcaaatgtcatgcgagatgagagattagtgccgatggaaaggcttatttcaagactttcgctcaaaaaatacgatgtagatgctccgatgaattttgatactgttattccttcacatcacgttgtcatgcaactaagtcagcatgttggtgccaaagcgatacctgtagtaaaggtaggagatattgtgaaagaaggagatctgataggcgatgtgcctaataataagctgggtgctaaattgcatgccagtattgacggcattataatagatgtaactgatgacagtattgttatcaaaccaagaggtgattttgatggacaaagcgataggattggttga

SEQ ID NO: 98 - protein, 182 aa, T. saccharolyticum
MDKAIGLVEYKSVATGITAADDMAKTADVEIIEAYTVCPGKYIVLLAGKLSAVNSAIEKGINQYSENVIDSFILGNPHETIYKAMSGTSVIEDVEALGIIETFSAASIILAADTAAKAAKVNLVEIRIARGMCGKSYLLLTGELAAVEASINAGCKALERTGMLLNKSIIPNPDRAIWDKII

SEQ ID NO: 99 - DNA, 549 bp, T. saccharolyticum
atggacaaagcgataggattggttgaatacaaatcagttgctacaggtataactgctgctgatgacatggctaaaactgctgatgtggaaataatagaagcatatacagtatgtccggggaaatacattgttctgttagctgggaaattaagtgcagttaattcggcgatagaaaagggcataaatcagtattcggaaaatgtcattgatagctttatattgggaaatccgcatgaaacaatatataaagctatgagtggcacgtctgtaattgaagatgtagaagcacttggtatcatagagacattttctgcagcatcaataatacttgcagcagatacggctgcaaaagctgcaaaagtgaatctggtagagataagaatagccagaggtatgtgcggcaagtcatatctactgcttacaggagaacttgctgctgttgaagcatctataaatgcaggatgcaaagctttggagagaacgggtatgcttttaaataagtctataatacccaatccagatagagctatttgggataagataatttaa

SEQ ID NO: 100 - protein, 425 aa, T. saccharolyticum
MYEAEKDKILNDYYNAKEIYAKFDIDIDKVLDKMKKIRISLHCWQGDDVTGFEKSANGLSGGGILATGNWPGRARNGEELRQDIEKALSLIPGKHKINLHAIYAETDGEFVDRDEINVEHFRKWIYWAKENGLGLDFNPTFFSHPKANDGYTLSSKDENIRKFWIQHGKRCREIANEIGRELKTQCVNNVWIPDGSKDLPANRIEHRKILKESLDEIFSVKYDKSNIVDSVESKLFGIGSESYVVGSHEFYMNYASRNDVMLCLDMGHFHPTENIADKISSILTFNDNLLIHVSRGVRWDSDHVVILNEDLLSLAKEIRRCDAYDKVYIALDFFDASINRIMAWVIGARATLKAILISLLEPVHLLMEEENKGNFGARLALMEEFKTLPFYSVWNKYCMDENVPIGTSWIDDVKEYEKEIVKNRA

SEQ ID NO: 101 - DNA, 1278 bp, T. saccharolyticum
atgtatgaagcagaaaaagataaaattttaaatgattattataatgcaaaagagatttatgcaaagtttgacatagatattgataaagtattagataaaatgaagaagattcgtatttcacttcactgctggcaaggcgatgatgtaactggattcgaaaaaagtgccaatggattaagcggtggaggtattttggcgacaggaaactggcctggtagagcaagaaatggtgaagaattaaggcaagacattgaaaaagccttaagccttataccaggcaaacacaaaatcaatttacatgccatttacgcagaaacggatggtgaatttgtagacagagatgaaataaacgtggagcatttcaggaaatggatttactgggcaaaagaaaatggccttggccttgacttcaatcctacgtttttttcgcatcctaaagcaaatgatggctatacgctttcaagcaaagatgaaaacataagaaaattttggatccaacatggtaaaagatgccgtgaaatcgcaaatgaaataggaagagagctaaaaactcaatgtgtgaataatgtttggattcctgatggttcaaaagatttgcctgctaataggattgaacacagaaaaatacttaaagaatctttagatgagatattttcagtaaaatatgacaaatcaaatatcgttgattctgttgaaagcaaattatttggcattggatctgaaagctatgtggttggttcacatgagttttatatgaactatgcgtcgagaaatgatgtaatgctgtgccttgatatgggacattttcatcctactgagaatattgctgataagatatcatcaatacttacattcaatgacaatttgttgattcatgtaagccgtggtgtccggtgggatagcgaccatgtagtcattttaaatgaagatttgctttcattagcaaaagaaataagaagatgtgatgcttatgacaaagtgtatattgcattagatttctttgatgcaagcataaataggataatggcatgggtaataggtgcaagagcgacgctaaaagccatattaatatcactattagagcctgtgcatctacttatggaagaggagaataaaggaaattttggtgcaagacttgctttgatggaggaattcaaaacattgccattttactctgtttggaacaaatactgcatggacgaaaatgtgcctattggtacatcgtggattgatgatgttaaagaatatgaaaaagaaattgtaaaaaatagggcttaa

SEQ ID NO: 102 - protein, 475 aa, T. saccharolyticum
MKDIVYNLAFDFGASSGRLMLSAFDGEKITIEEIYRFPNEPVKLGQSFYWDFLRLFHELKNGLKIASKRKIKISGIGIDTWGVDYGLLDKNDQLISNPFHYRDKRTDGIIKDFENMALLEEIYNVTGIQFMEFNTIFQLYCDYKKRPELLDNAKTLLFIPDLFNFYLTNEKYNEYTVASTSQMLDANKKDWANDLIEKLNLPEGIFQKILMPGNTIGYLTKEIQEETGLSEVPVISVGSHDTASAVAGTPIENGSSAYLICGTWSLLGVESEKPIINENTKKYNFTNEGGVEGLIRLLKNINGLWIIQQLKQSWNSNGIKIGFPEISQMASKAEHEEFIINPDDKLFIAPDDMAEAIRQYCTKTGQGLPQNIGDIARAAYNGIVEQYKNCLNNLEDIVGQEIDNIHMVGGGIQDKFLCKLTADVTGKKVITGPVEASIYGNVIVQLMALGYIKDLREGRKIIKNSIENDEEMFAK

SEQ ID NO: 103 - DNA, 1428 bp, T. saccharolyticum
atgaaagatattgtgtataatctggcttttgattttggagcttcaagtggccgtcttatgctatccgcgtttgatggcgaaaaaatcacaattgaagagatttatagatttccaaatgagccagtcaagctgggacaatcattttattgggattttttaaggctttttcacgaattaaaaaacggattaaaaatagcatcaaagaggaaaatcaaaatatccggcattggtatagacacttggggtgtcgattatggattgcttgataaaaatgatcaattgatttcaaatccttttcattacagagataaaagaacggatggcataataaaagattttgaaaatatggcgttactggaggaaatctacaacgtaactggtatacagtttatggaatttaatacaatattccaattgtattgcgattataaaaagcgtccagaattattggataatgcaaagacattgttgtttattccagatttatttaacttttatttgacaaatgagaaatacaatgaatatactgttgcatccacatcgcaaatgttggatgctaacaagaaagattgggcaaatgatcttatagaaaagttaaatttgccagaaggtatttttcaaaagatactgatgccaggaaatacaattggttatctaacaaaagaaattcaagaagaaacaggattgtctgaagttcccgtgatttctgttggcagccatgatacggcatcagcagttgcaggtacacctattgaaaacggttcaagtgcttatttgatttgtggtacttggtcattattaggtgttgaaagtgaaaaacctataataaatgaaaatacaaagaagtacaattttacaaatgaaggcggtgtcgaaggccttataaggctacttaaaaatattaatggtctgtggataattcagcaattaaaacaaagttggaattcaaatggcattaaaataggatttccagaaatcagccagatggcatctaaagcagagcacgaagaatttatcataaatcctgatgacaaattgtttatagctccagatgatatggctgaggcgataaggcaatattgtacaaaaacaggacagggtttgccgcagaatattggcgacatagcaagagccgcttacaatggtatagttgaacaatacaaaaattgcttaaacaatttagaagatattgtagggcaagaaatagataatattcacatggttggtggtgggatacaggataagttcctgtgcaagctgactgcagatgttacagggaaaaaagtcataacaggccctgtagaagcttcaatctatggcaatgtgatagtccagcttatggcattgggatatataaaagacttgagagaaggaagaaagataataaagaattctatagagaatgatgaagagatgtttgctaaatag

SEQ ID NO: 104 - protein, 254 aa, T. saccharolyticum
VSNIYTLVVVEDEYEIRTGLVNCFPWNKMGFVVAEEFENGGECFEYLCKNKVDTILCDIKMPVMSGIELAKKIFESNISTKIVIISGYTDFEYARQALRYGVKDYIVKPTKYNEIIDVFSRIKKELDNENTKEILNNSCNNEIDQYSSIISIIEKYVDEHYRDVTLEDVAKVVYMNPYYLSKYFKQKTGMNFSDYITEVRMKKAVEFLKNPLYKTYEISYMIGYKNPKNFTRAFKKYYKKSPREFVNSAINFKE

SEQ ID NO: 105 - DNA, 765 bp, T. saccharolyticum
gtgtctaatatttatacgcttgtagtagtagaagatgaatatgagataagaacaggattagttaactgctttccatggaacaaaatgggttttgttgttgcagaagaatttgaaaatggaggagaatgttttgagtatttgtgtaaaaataaggttgatacaattttatgtgatataaaaatgccagttatgtctggtatagagttggcaaagaaaatttttgaaagtaatataagcactaaaatagttataatcagtggttatactgattttgaatatgccagacaggcgttaagatatggtgttaaagattatatagtaaaacctactaaatataatgaaataattgatgttttcagcagaataaaaaaagaattagacaatgaaaatacaaaggaaatattgaataactcatgtaacaatgaaattgatcagtacagcagcataatttcaatcatagaaaaatatgttgatgaacattacagagatgtgacattggaagatgtagctaaagtagtttatatgaatccgtattatttaagcaaatattttaaacaaaaaaccggtatgaatttttctgattatataactgaggtcagaatgaaaaaagctgtagagtttctaaaaaatcctttgtataaaacttatgaaataagttatatgattggatataaaaatccaaaaaattttactagagcatttaaaaaatattataaaaaatccccaagagaatttgtaaattcagcaataaattttaaggaatga

SEQ ID NO: 106 - protein, 588 aa, T. saccharolyticum
MRELNNKFFYKNLFVLALPLILIVIVLGSFSILITERYVRDEIYKNSREILKQSSNDLSILFNDINKIYLTFGTNKDVTLYLERILNTNKYSLDDMWHLSMIESLFDSTSFSEPYIQSIYLYFNNPNKNFLVTGNGINSVTNYIDNKWYDSFLNAPKDEISWIEVRNLKMYSFDKKGIKVLSIYKKIANFNGDKIDGVLVLNIYLDYIENLLNTSTIFPDQKILILDAHDNLICQNINGNFTGKIDLDNYSKANIITKLESPNYNIKYVSIVPKKYLYEVPIKLLKMTLVLLLTSIFFVILITFRITKRNYENVNKILKIIEAEKTNEIFPEIPVESRDEYSYIIYNIINSYIEKSQLKMELAEKKYKMKAMELLALQSQISPHFLSNALEIIYLRALSYTNGPNDVTKMIENLSQILKYLLSNPNETVTVKEEIENTKAYIQILKVRYRDKFKVNLIYDESILSCLMMKLMLQHLIENSIKHGLKKKNYEGSIKIKIKAVDKKKIKISVIDNGIGMSKERLNYVKRILDSDFDFYEHIGLMNTNERLKLLYGKDCEILIRSKLNIGTAVYIIFPYQLKNQNNDDYNK

SEQ ID NO: 107 - DNA, 1767 bp, T. saccharolyticum
atgagagaattaaacaataaatttttttataaaaatctttttgttttggcattgccattaattttaattgttattgtattaggttcattttcaatattaataacagaaagatatgttagagatgaaatatacaaaaatagtagagaaatattaaagcaaagcagtaatgatttgtcaattttatttaatgatataaataaaatttatttaacatttggaacaaacaaagatgtgacattgtatttggaaaggatcttaaatacaaataaatattctttagatgatatgtggcatcttagcatgatagaaagtttatttgattctacgtcgttttcagaaccttatatacaatcaatttatttgtattttaacaatcctaataaaaattttttagtgacaggaaatggtattaattctgtaacaaattatattgataataaatggtatgacagctttttaaatgcaccaaaagatgagatttcttggatagaggttagaaatttaaaaatgtatagtttcgataaaaaggggataaaagtcctaagtatatacaaaaaaattgcaaactttaacggggataaaattgatggtgtgcttgtactaaatatatatttggactatattgaaaatttgctaaatacttcaacaatatttcctgaccaaaaaattcttatattagatgcccacgacaatttaatatgtcaaaatattaatgggaatttcactgggaagatagacttagataattatagcaaagcaaacatcataacaaaattagaatctccaaattataatataaaatatgtatctattgttcctaaaaaatacctttatgaagttcctataaagcttttaaagatgactttagttttacttttgacgtcaattttttttgtgatattgataacatttagaatcactaaacgaaattacgaaaatgtaaataaaatattaaagattatagaggcagaaaagacaaatgagatatttccagaaattccagtagaaagtagagatgagtacagctatataatttacaacattattaatagttatattgaaaaaagtcaattgaaaatggaattagcagaaaagaagtataaaatgaaagcaatggagttattagcactgcaatcgcaaattagtcctcattttttgtctaatgcgttggagattatttatcttagggcattgtcatacacaaacggtcctaatgatgtcacaaaaatgattgaaaatttgtcacagattttaaagtatttgttaagtaatccaaatgaaacagtaactgtaaaagaagaaattgaaaatacaaaggcatatatacaaatattgaaggtcaggtatagagataaatttaaagtaaatctaatttatgatgaaagtattttatcatgtctcatgatgaaactgatgctgcaacatttaatagaaaattctataaaacatgggcttaagaagaaaaattatgaaggatcaataaaaatcaaaataaaagcagttgataaaaagaaaataaaaatttcagtaatcgataatggcataggaatgtccaaagagaggctaaattatgtaaaaagaattcttgactctgacttcgatttttatgaacatattggactaatgaatacaaatgaacggttaaaacttctctatgggaaagattgtgaaatattaataagaagtaaattgaatattggtactgccgtatatataatttttccatatcaattaaaaaatcagaataatgatgattataataagtga

SEQ ID NO: 108 - protein, 687 aa, T. saccharolyticum
MGINRYDLVKRHNVILEKADIENPLSVGNGEIAFTADITGMQTFIDDYKSIPLCTMSQWGFHTTPAQNDKGYYTLEDLNLKYYDAFDRKVGYVTSAENQENVFNWLRSNPHRINLGNIGLNIILDDGTKAELKDIFEIHQVLDLWNGILISDFKVEKVPVHVETFCHPYEDMINFSVESELLKQNKIYIEVKFPYGAANISGSDWDRNDRHDTNVVDYGRDFVELLRTVDEDVYFVKIEYSKGVYLNRIGENHFALKQKEYNGRIEFSCLFSKQKPLKCLHSFSESKRMCKEYWNSFWRGGGAIDFSKCEDKRAFELERRVILSQYLTAIQCSGSMPPQETGLTCNSWYGKFHLEMHWWHAVHFALWGRMPLLSRSIWWYRSIFNVSRDIARKQGYKGVRWPKMVGPDGRDSPSPIGPLLVWQQPHLIYYSELFFRENPTEETLDMFKDIVINTADFIASFVAYDRKNDRYILAPPLIPAQENHDPNVTLNPVFELEYFSFALEIAVKWIERLGLNVNQEWNEIRFKLANLPSKDGVYISHEKCINTYEKFNFDHPSMLAALGMLPGRKVDKETMRRTLHRVLKEWKFEEMWGWDFPMMAMTATRLGEPETAINILLMDSPKNTYMVNGHNNQIPNKELPVYLPGNGGLLAAMALMTAGWDGNSQSTPGFPKNGMWNVEWEGLKAMI

SEQ ID NO: 109 - DNA, 2064 bp, T. saccharolyticum
atgggaattaacagatatgatcttgtaaaaaggcataatgtaattttggaaaaagcagatatcgaaaatccattgtcagtaggtaatggagaaattgcttttacagctgatataacgggaatgcaaacttttattgatgactataagagcattcctttatgtaccatgtcacagtgggggtttcatactacgccggcacagaatgataagggctattatactttggaagatttgaacctcaagtattacgatgcatttgaccgaaaggttggatatgtaacatcagcagaaaatcaagagaatgtatttaattggttgaggagtaatcctcatagaattaatttaggtaatataggattaaatataattcttgatgatggcacaaaagcagaattgaaagatattttcgaaatacaccaagtattagatttgtggaacggaatattgataagtgactttaaagtcgaaaaagtccctgttcacgttgagactttttgccatccatatgaagatatgataaatttttctgttgaatcagaactgctaaaacaaaataaaatttatattgaagtaaaatttccatatggtgcggccaatatatcaggctccgattgggatagaaatgatagacatgatacaaatgtggttgattatggcagagattttgtcgaattattgagaactgtcgatgaagatgtttattttgtaaaaatagagtactcaaaaggcgtttatttaaatagaatcggggaaaatcattttgcattaaagcaaaaagagtataatgggagaatagaattttcgtgcttgttttcgaagcaaaaacctcttaagtgcttgcattcatttagtgaaagcaaaaggatgtgtaaagaatattggaatagcttttggagaggaggtggtgcaatagatttttcaaagtgtgaggataaaagagcttttgaattggagagaagggtaatactttcgcaatatcttacagctattcaatgttcgggttctatgccgccgcaagaaacagggctcacctgtaatagctggtatggtaaatttcatttggaaatgcattggtggcatgctgtacattttgctttatggggtagaatgcctttgctgagtagaagtatatggtggtacaggagcattttcaatgtatcacgtgacattgcgagaaagcaaggatacaaaggtgtacgctggcctaaaatggttggaccagatggaagggatagcccttctccgataggaccattgcttgtttggcagcagcctcatcttatatattacagtgaactgttttttagagaaaatcctacggaagaaacattagatatgtttaaagacatagtaattaatactgctgattttattgcatcatttgttgcatatgatagaaaaaatgatagatatatacttgcgccacctttgattccagcacaagaaaatcatgatcctaacgttacattaaatccggtatttgaattggagtatttttcgtttgcgctggaaatagcagttaaatggattgaaaggttaggactaaatgtgaaccaagagtggaatgaaatacgttttaaattagctaatttaccttcaaaagacggtgtatatatatcgcatgaaaaatgtattaacacttatgagaaatttaattttgaccatccatctatgcttgcagcattggggatgctaccaggccgcaaggttgataaagaaactatgagaaggactttacatagagtattaaaagagtggaaatttgaggaaatgtggggttgggattttccgatgatggctatgactgcaacaagattaggcgaaccggagacagcaataaatattcttttgatggattcaccaaaaaatacttatatggtaaatggccataataaccaaataccgaataaagaactaccagtatatttgcctggaaatggtggactattggcggcaatggccctcatgacagctggttgggatgggaatagccaaagcacacctggatttcctaaaaatgggatgtggaatgttgaatgggaagggttaaaagcgatgatatga

SEQ ID NO: 110 - protein, 293 aa, T. saccharolyticum
MIKRKDLYIRDPFVVPVPNEKIYYMFGTTDINCWNDEKATGFDYYKSSDLENFEGPFIAFRPDKNFIWDKNFWAPEVHKYNDMYYMFATFFADGRNRGTQILVSEKISGPYRPWSIEPVTPKDWMCLDGTFYVDENGEPWMIFCHEWVQIYDGEICAVRLSKDLKTTIGNPITLFKASSANWTRSIKKIKDHECYVTDGPFIYRSEEGKLYMLWSSFIENNIYAVGISLSRTGKITGPWVHSENPIFAGDGGHGMIFKTFEGNLTLAVHTPNKRKEERPLFITLEKSVLNDTL

SEQ ID NO: 111 - DNA, 882 bp, T. saccharolyticum
atgataaaacgaaaggatctttatatacgtgatccatttgtagttccagtaccgaatgaaaaaatatattatatgtttggaactactgatataaattgctggaatgatgagaaagcaactggatttgattactataaatcatctgatttagaaaattttgaaggaccttttattgcatttagaccagataaaaactttatttgggataaaaatttttgggctccagaagtgcacaaatacaatgacatgtattatatgtttgctacatttttcgctgatggcagaaatagaggaacgcaaattttagtatctgaaaaaataagtgggccatatagaccatggagtattgaaccggtgacgccgaaggattggatgtgtttagatgggactttttatgtagatgagaatggggaaccctggatgatattttgccatgaatgggtacaaatatatgatggggaaatttgtgctgtaagattgtcgaaagatttaaaaacaacgataggaaatcctattacactttttaaagcttccagtgctaattggacaagaagtattaaaaagattaaagatcatgaatgctacgttacggatggcccttttatttataggtctgaagagggaaagctttatatgttgtggtccagttttattgaaaacaatatatacgctgttggtatatcattatcgagaacaggcaaaataaccggcccgtgggtacacagtgaaaatccaattttcgcaggtgatggtgggcatggtatgatatttaagacctttgaagggaatctaacattggcagtacacacacctaataaaaggaaagaagaacggcccctttttataactttagaaaaatctgtgcttaatgataccttataa

SEQ ID NO: 112 - protein, 442 aa, T. saccharolyticum
MFKKITSLLISLLLIISLVTGCSSSSNSSSSSKNSSENNTSPKTVTLRFMWWGGDARHKATLDAISLYEKEHPNVKINAEYGGVTDYLQKLITQLSSGTAPDLIQIDVTWLQQLFSQGDFFADLSKLKDINVNAFDQNFLKNYCYVNNKLIGLPTGINNSAMYINKDFFNKFGIDDKTVWTWDNLLQTAKMVHEKDKNAYLLDADSTICDYILVTYVGQKTGNQWVKDDYTLGFDKQTLTEAFKYLNDLFEVGAIEPFSQSAPYEGKPDQNPMWLNGQTGMLWNWSSIYAGVKANIKNLSLALPPIDPNAKQTGIVVRPSQLIAINKDSKNIDEAAKFLNWFFTNTDAIKTLKDVRGVPATADARKILSENNLLDSTLTDNANQAMEKMAPPENGISGNQELEKINTDIIQELAYKKITPEQAADELINTYKQKLPELKSQQ

SEQ ID NO: 113 - DNA, 1329 bp, T. saccharolyticum
atgtttaaaaaaattacatctctgttaatatcgcttcttttgataatttcattagttacaggatgtagcagttcttcgaattcttcgagttcatcgaaaaatagttctgaaaataataccagcccaaaaaccgtaacattaagatttatgtggtggggtggagatgccagacataaagcaacacttgatgccataagtctttatgaaaaagaacatcccaatgtaaagattaatgctgaatatggcggcgttactgactatctccaaaagctgataactcaattaagcagtggtacagcacctgatcttatacaaatagatgtaacatggttgcagcaactttttagccaaggtgatttttttgcagatttaagtaagttaaaagatatcaatgtgaatgcatttgatcaaaattttcttaaaaattattgctatgtcaacaataagttgataggtttgcctacaggaataaacaattcggcaatgtatattaacaaagacttttttaataaatttggcatagacgataagacggtttggacatgggataatctcttgcaaaccgctaagatggtgcatgaaaaggataaaaatgcttatcttttagatgctgattctactatttgtgattatatattggtcacatacgtggggcaaaaaactggaaatcagtgggtgaaagatgattacactttaggttttgataaacaaacattgacagaggcattcaaatatttaaacgatttgttcgaagtaggcgctatagagccattttctcaaagtgctccatacgaaggaaaacctgatcaaaatcctatgtggcttaatggtcaaacgggtatgctttggaactggtcatctatatatgctggtgtaaaagcaaacataaagaacctgtcattggcattgccacctattgaccctaatgcaaaacagacaggcatagttgtaagaccatcacagcttattgctattaacaaggattctaaaaatatcgatgaagcagcaaaatttttaaattggttctttacgaatacagatgctataaaaacacttaaagatgtcagaggagttccagctaccgcagatgcacgcaaaattttatcagaaaataatttgttggattcgactttaactgataatgcaaatcaagctatggaaaagatggcacctcctgaaaacggtataagtggtaatcaagagttagaaaagataaatactgatatcatacaagaactggcttataaaaagataacgccagagcaggctgctgatgaattgataaatacttataaacagaaacttccagaattaaaaagccagcaataa

SEQ ID NO: 114 - protein, 295 aa, T. saccharolyticum
MSYNKKRNLMGYLYISPWIIGFLIFTLYPFAMTFIYSFCNYSITKSPVFIGLGNYITMFTKDMYFWPSLINTIKYVLMTVPLKLCFALFVAMILNIDIKGVNVFRTTYYLPSIFGGSVALSVIWKFLFMDNGIMNKFLSYFHIHGPSWLGNPHISLFTISLLSVWEFGSSMVIFLAALKQVPNELYEASMLDGASKIRRFFSITLPMISPVLLFNLVMQTINAFQEFTGPYVITGGGPMNSTYVYSMLIYDNAFRYFRMGYSSALSWILFLLILIVTVIIFKSSNTWVYYENGGR

SEQ ID NO: 115 - DNA, 888 bp, T. saccharolyticum
atgagttataataaaaagagaaatttgatggggtatttatatattagtccatggattataggctttttaatatttactctgtatccatttgctatgacttttatctattcattttgtaactacagtattacaaaatcacctgtatttattggattaggcaattatataactatgtttactaaagatatgtatttttggccatctttaattaatactataaaatatgtattaatgacagttcctttaaaattatgttttgcactttttgttgcaatgatcttaaatattgatattaaaggagttaatgtgtttagaacaacttattatctgccttctatttttggaggaagtgttgctttatctgttatatggaaatttttattcatggataatggtattatgaataaatttctttcatactttcatatacacgggccaagttggcttggaaacccacacatatcattatttactataagtttattgtcagtgtgggaatttgggtcttctatggtaatatttttggcagccctaaaacaggtcccgaatgagttgtatgaagcatctatgttagatggtgcaagcaaaataagaaggtttttctcaataactttacctatgatatcgcctgtgctattatttaatttggttatgcagactataaatgcttttcaggaatttacaggtccatacgtgataactggtggaggaccgatgaactctacttatgtgtacagtatgttgatttatgataatgcgtttaggtattttaggatgggttattcatctgccttgtcttggattttatttttgttaatattgattgttacagttataatatttaaatcttcaaatacatgggtgtattacgaaaatggaggtagatga

SEQ ID NO: 116 - protein, 285 aa, T. saccharolyticum
MKAKNSQNNDIIRKVFIYVFLVAFGIFMIYPLLWVFASSFKSNDEIFKSISLIPKHIVTNSYFEGWKGTGQYSFGTFILNSITLVVPVVVFTAISSTIVAYGFARFEFPLKTILFTLMISTMMLPGTAVLIPRYILFNWLGWINTYKPFIVPALFGTTPFFIFMMVQFLRGLPKELEESATIDGCNSFQILMKILIPLCKPAIISMCIFQFIWTWNDFFNPLIYINSVEKYTVSLGLNMTIDGTSVVNWNQIMAMTIISMIPSIIIFFSAQKYFVEGIATTGLKN

SEQ ID NO: 117 - DNA, 858 bp, T. saccharolyticum
atgaaagcaaagaatagtcaaaataacgatataatcagaaaagtatttatatatgttttcttggtggcttttggtattttcatgatatatcctttactttgggtttttgcatcatcatttaaatcaaatgatgaaatctttaaatcgataagccttataccaaaacacattgtgacaaattcatattttgaaggatggaaaggtacgggacaatactcttttggtacatttattttaaacagcattacgcttgttgtacctgttgttgtatttactgctatatcatcaacaattgtagcctatggatttgcaagatttgagtttccgcttaaaactattttgtttactttgatgatatctactatgatgttgccgggcactgcagttttgataccaagatatatattgtttaattggttaggctggataaacacttataaaccatttattgttcccgctttgttcggaacaacgccttttttcatttttatgatggttcaatttttgagaggtcttcctaaagaattagaagaatcggctacaattgatggttgcaattcatttcaaatacttatgaagattttaataccattgtgtaaacctgcaattatttctatgtgtatatttcagttcatttggacttggaatgacttttttaatccattgatatatatcaacagtgtagaaaaatatacagtttctctcgggcttaatatgacaattgatgggacttcagttgtaaattggaaccaaataatggcaatgacaattatttcaatgataccgagcatcataatatttttttcagcgcaaaaatacttcgttgaaggtattgcaacaactggattaaagaactaa

SEQ ID NO: 118 - protein, 465 aa, T. saccharolyticum
MRYTDGKVHDITIAYIGGGSRGWAWNLMTDLAKEESISGTVKLYDIDYDAAHDNEIIGNALSMRQDVKGKWLYKACETLEESLKGADFVIISILPGTFDEMESDVHAPEKYGIYQSVGDTVGPGGIVRALRTIPMFVDIANAIKEHCPDAWVINYTNPMTLCVRTLYEIFPQIKAFGCCHEVFGTQKLLSRALQDIEGIENVPREEIKINVLGINHFTWIDNARYKDIDLMYVYKQFVNKYYESGFVSDANNNWMNNSFVSAERVKFDLFLRYGVIAAAGDRHLAEFVPGYWYLKDPETVREWMFGLTTVSWRKEDLKRRLERSKRLKTGEEKFELKETGEEGVRQIKALLGLGDLVTNVNMPNHGQIEGIPYGAVVETNALFSGNKLKPVLSGKLPDNVNSLVLRQVYNQETTLKAALKRDFDLAFSAFVNDPLVTISLKDAKKLFKEMLENTKKYLDGWKIKA

SEQ ID NO: 119 - DNA, 1398 bp, T. saccharolyticum
atgagatatacagatggaaaggttcatgacattactattgcttatatcggtggtggttcaagaggatgggcgtggaatttaatgactgacttagcaaaagaggaaagtatttctggtacagtaaagttatacgacatagattacgatgcggcacatgacaatgagataataggcaatgctttatcaatgagacaggatgttaaaggcaaatggctttataaagcttgtgagacgttagaagagtcactaaaaggtgctgattttgtcataatatctattttgccaggtacgttcgacgagatggaatctgatgttcatgcaccagaaaagtatggcatttatcagtcagtaggtgatacagtaggacctggtggaatagtcagagctttaaggacgattccgatgtttgtggacattgccaatgcgattaaagagcattgtccagatgcatgggtcataaattatacaaatcctatgacactttgtgtaaggacattgtatgaaattttccctcaaattaaagcatttggatgctgccatgaagtttttggcacacagaagctattatctcgtgctctgcaggatatagaaggcattgaaaatgttccgagggaagagataaagataaatgttttaggtataaatcattttacgtggatcgacaatgcaaggtacaaagacatagatttaatgtatgtttataaacaatttgtgaataagtactatgaaagcggatttgtcagcgatgctaacaataattggatgaacaattcatttgtatctgcagagagagtaaagtttgatctgtttttgaggtatggagtaatagctgcagcgggagatagacatctggcggaatttgtgccgggatattggtatttaaaagatccagagacagtcagagaatggatgtttggcttaacgactgtaagttggagaaaagaagacttaaaacgcaggcttgaaagaagtaaaaggcttaagacaggtgaggaaaaatttgagttaaaggaaacaggcgaagaaggtgttaggcaaattaaagcactattaggcttaggcgatttagtgactaatgtcaacatgccgaaccatggacagattgaaggaataccatacggtgcggtagttgaaacaaacgctttattttcaggtaataaactaaagcctgtattatcaggaaaattgcctgacaatgtaaacagcctcgtgttaaggcaagtatacaaccaagaaacgacgttgaaagctgctttaaagagagattttgatttggcttttagtgcttttgtaaatgatccacttgttacaatatctttaaaagatgcaaaaaaattatttaaggaaatgcttgaaaatacgaagaaatatctagatggatggaaaataaaagcttga

SEQ ID NO: 120 - protein, 392 aa, C. acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 121 - protein, 218 aa, C. acetobutylicum
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPA

SEQ ID NO: 122 - protein, 221 aa, C. acetobutylicum
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAV

SEQ ID NO: 123 - protein, 244 aa, C. acetobutylicum
MLKDEVIKQISTPLTSPAFPRGPYKFHNREYFNIVYRTDMDALRKVVPEPLEIDEPLVRFEIMAMHDTSGLGCYTESGQAIPVSFNGVKGDYLHMMYLDNEPAIAVGRELSAYPKKLGYPKLFVDSDTLVGTLDYGKLRVATATMGYKHKALDANEAKDQICRPNYMLKIIPNYDGSPRICELINAKITDVTVHEAWTGPTRLQLFDHAMAPLNDLPVKEIVSSSHILADIILPRAEVIYDYLK

SEQ ID NO: 124 - protein, 244 aa, C. acetobutylicum; identical to SEQ ID NO: 123

SEQ ID NO: 125 - protein, 899 aa, M. thermoacetica
MVNLTIDGQRVTAPEGMTILEVARENGIHIPTLCHHPKLRPLGYCRLCLVDIEGAAKPMTACNTPVAEGMVIRTSTPVIEEMRKGIIEMLLSLHPEDCLTCEKAGNCQLQDCAYTYGVKHGELPVKREELPVLKENPFIVRDYNKCIVCGRCVRACQEVQVQRVVDLVGKGSAARVGATKAGAEVSLEEGGCVFCGNCVQVCPVGALTEKAGLGQGREWEFKKVRSICSYCGVGCNLTLYVKDGKVVKVRGYENPEVNNGWLCVKGRFGFDYIHNPDRITRPLIREGDREKGYFREASWEEALALVSQKLTQIKGSYGSEALGFLCSAKCTNEENYLLQKLARGVLGTNNVDHCARLHSSTVAGLATTFGSGAMTNSIADIASADCIFVIGSNTTENHPVIALKVKEAVRRGARLIVADPRRIELVNFSYLWLRQKPGTDLALLNGLLHVIIKEELYDKEFIAQRTEGFEALKLAVEEYTPAKVSEVTGVPAGDIIEAARTYARGPSSTILYAMGITQHITGTANVMALANLAMACGQVGKEGSGVNPLRGQSNVQGACDMGGLPNVLPGYQPVTDPGVRHKFSEAWGVPDLPGEPGLTLMEMMAAAQEGKLKGMYILGENPVLTDPDVSHVKEALKNLEFLVVQDIFLTETARMADVVLPGASFAEKEGTFTSTERRVQLLHKAIEPPGEARPDWLILNDLLLLMGYPRKYSSPGEIMQEIAGLTPSYAGITYERLEDKGLQWPVLSLEHPGTPVLHREKFSRGYGQFQVVHYRPPAEEPDEEYPFLFTTGRNLYHYHTVISRKSRGLEEMCPAPVVEINDNDAARLGIREGEMIEIVSRRGKVRVKALVTDRIPRGQVFMNFHFHEAAANLLTIAALDPVAKIPEYKTCAVAIKVKK

SEQ ID NO: 126 - protein, 152 aa, Oryza sativa
MELTTRTIAERKHIALVAHDHRKQALLEWVESHKTILAQHQLYATGTTGNLIQRASGIPVTSMLSGPMGGDQQVGALIAEGKIDMLIFFWDPLNAVPHDPDVKALLRLATVWNIPVATNRSTADFLIDSPLFKSEVAIAIPDYQRYLQDRLK

SEQ ID NO: 127 - protein, 350 aa, T. saccharolyticum; identical to SEQ ID NO: 47

SEQ ID NO: 128 - protein, 365 aa, Pseudomonas putida
MDRAIQSPGKYVQGADALQRLGDYLKPLADSWLVIADKFVLGFAEDTIRQSLSKAGLAMDIVAFNGECSQGEVDRLCQLATQNGRSAIVGIGGGKTLDTAKAVAFFQKVPVAVAPTIASTDAPCSALSVLYTDEGEFDRYLMLPTNPALVVVDTAIVARAPARLLAAGIGDALATWFEARAASRSSAATMAGGPATQTALNLARFCYDTLLEEGEKAMLAVQAQVVTPALERIVEANTYLSGVGFESGGVAAAHAVHNGLTAVAETHHFYHGEKVAFGVLVQLALENASNAEMQEVMSLCHAVGLPITLAQLDITEDIPTKMRAVAELACAPGETIHNMPGGVTVEQVYGALLVADQLGQHFLEF

SEQ ID NO: 129 - protein, 564 aa, T. saccharolyticum; identical to SEQ ID NO: 48

SEQ ID NO: 130 - protein, 218 aa, C. acetobutylicum; identical to SEQ ID NO: 121

SEQ ID NO: 131 - protein, 221 aa, C. acetobutylicum; identical to SEQ ID NO: 122

SEQ ID NO: 132 - protein, 244 aa, C. acetobutylicum; identical to SEQ ID NO: 123

SEQ ID NO: 133 - protein, 246 aa, Escherichia coli
MSVIGRIHSFESCGTVDGPGIRFITFFQGCLMRCLYCHNRDTWDTHGGKEVTVEDLMKEVVTYRHFMNASGGGVTASGGEAILQAEFVRDWFRACKKEGIHTCLDTNGFVRRYDPVIDELLEVTDLVMLDLKQMNDEIHQNLVGVSNHRTLEFAKYLANKNVKVWIRYVVVPGWSDDDDSAHRLGEFTRDMGNVEKIELLPYHELGKHKWVAMGEEYKLDGVKPPKKETMERVKGILEQYGHKVMF

SEQ ID NO: 134 - protein, 760 aa, Escherichia coli
MSELNEKLATAWEGFTKGDWQNEVNVRDFIQKNYTPYEGDESFLAGATEATTTLWDKVMEGVKLENRTHAPVDFDTAVASTITSHDAGYINKQLEKIVGLQTEAPLKRALIPFGGIKMIEGSCKAYNRELDPMIKKIFTEYRKTHNQGVFDVYTPDILRCRKSGVLTGLPDAYGRGRIIGDYRRVALYGIDYLMKDKLAQFTSLQADLENGVNLEQTIRLREEIAEQHRALGQMKEMAAKYGYDISGPATNAQEAIQWTYFGYLAAVKSQNGAAMSFGRTSTFLDVYIERDLKAGKITEQEAQEMVDHLVMKLRMVRFLRTPEYDELFSGDPIWATESIGGMGLDGRTLVTKNSFRFLNTLYTMGPSPEPNMTILWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNYDEVMERMDHFMDWLAKQYITALNIIHYMHDKYSYEASLMALHDRDVIRTMACGIAGLSVAADSLSAIKYAKVKPIRDEDGLAIDFEIEGEYPQFGNNDPRVDDLAVDLVERFMKKIQKLHTYRDAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMENPEKYPQLTIRVSGYAVRFNSLTKEQQQDVITRTFTQSM

SEQ ID NO: 135 - protein, 398 aa, Saccharomyces cerevisiae
MSQNVYIVSTARTPIGSFQGSLSSKTAVELGAVALKGALAKVPELDASKDFDEIIFGNVLSANLGQAPARQVALAAGLSNHIVASTVNKVCASAMKAIILGAQSIKCGNADVVVAGGCESMTNAPYYMPAARAGAKFGQTVLVDGVERDGLNDAYDGLAMGVHAEKCARDWDITREQQDNFAIESYQKSQKSQKEGKFDNEIVPVTIKGFRGKPDTQVTKDEEPARLHVEKLRSARTVFQKENGTVTAANASPINDGAAAVILVSEKVLKEKNLKPLAIIKGWGEAAHQPADFTWAPSLAVPKALKHAGIEDINSVDYFEFNEAFSVVGLVNTKILKLDPSKVNVYGGAVALGHPLGCSGARVVVTLLSILQQEGGKIGVAAICNGGGGASSIVIEKI

SEQ ID NO: 136 - protein, 348 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 137 - protein, 348 aa, Saccharomyces cerevisiae
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 138 - protein, 375 aa, Saccharomyces cerevisiae [sequence omitted]

SEQ ID NO: 139 - protein, 382 aa, Saccharomyces cerevisiae
MSSVTGFYIPPISFFGEGALEETADYIKNKDYKKALIVTDPGIAAIGLSGRVQKMLEERDLNVAIYDKTQPNPNIANVTAGLKVLKEQNSEIVVSIGGGSAHDNAKAIALLATNGGEIGDYEGVNQSKKAALPLFAINTTAGTASEMTRFTIISNEEKKIKMAIIDNNVTPAVAVNDPSTMFGLPPALTAATGLDALTHCIEAYVSTASNPITDACALKGIDLINESLVAAYKDGKDKKARTDMCYAEYLAGMAFNNASLGYVHALAHQLGGFYHLPHGVCNAVLLPHVQEANMQCPKAKKRLGEIALHFGASQEDPEETIKALHVLNRTMNIPRNLKELGVKTEDFEILAEHAMHDACHLTNPVQFTKEQVVAIIKKAYEY

SEQ ID NO: 140 - protein, 351 aa, Saccharomyces cerevisiae
MPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSK

SEQ ID NO: 141 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 142 - protein, 361 aa, Saccharomyces cerevisiae
MLYPEKFQGIGISNAKDWKHPKLVSFDPKPFGDHDVDVEIEACGICGSDFHIAVGNWGPVPENQILGHEIIGRVVKVGSKCHTGVKIGDRVGVGAQALACFECERCKSDNEQYCTNDHVLTMWTPYKDGYISQGGFASHVRLHEHFAIQIPENIPSPLAAPLLCGGITVFSPLLRNGCGPGKRVGIVGIGGIGHMGILLAKAMGAEVYAFSRGHSKREDSMKLGADHYIAMLEDKGWTEQYSNALDLLVVCSSSLSKVNFDSIVKIMKIGGSIVSIAAPEVNEKLVLKPLGLMGVSISSSAIGSRKEIEQLLKLVSEKNVKIWVEKLPISEEGVSHAFTRMESGDVKYRFTLVDYDKKFHK

SEQ ID NO: 143 - protein, 417 aa, Saccharomyces cerevisiae
MRALAYFGKGNIRFTNHLKEPHIVAPDELVIDIEWCGICGTDLHEYTDGPIFFPEDGHTHEISHNPLPQAMGHEMAGTVLEVGPGVKNLKVGDKVVVEPTGTCRDRYRWPLSPNVDKEWCAACKKGYYNICSYLGLCGAGVQSGGFAERVVMNESHCYKVPDFVPLDVAALIQPLAVCWHAIRVCEFKAGSTALIIGAGPIGLGTILALNAAGCKDIVVSEPAKVRRELAEKMGARVYDPTAHAAKESIDYLRSIADGGDGFDYTFDCSGLEVTLNAAIQCLTFRGTAVNLAMWGHHKIQFSPMDITLHERKYTGSMCYTHHDFEAVIEALEEGRIDIDRARHMITGRVNIEDGLDGAIMKLINEKESTIKIILTPNNHGELNREADNEKKEISELSSRKDQERLRESINEAKLRHT

SEQ ID NO: 144 - protein, 386 aa, Saccharomyces cerevisiae
MSAATVGKPIKCIAAVAYDAKKPLSVEEITVDAPKAHEVRIKIEYTAVCHTDAYTLSGSDPEGLFPCVLGHEGAGIVESVGDDVITVKPGDHVIALYTAECGKCKFCTSGKTNLCGAVRATQGKGVMPDGTTRFHNAKGEDIYHFMGCSTFSEYTVVADVSVVAIDPKAPLDAACLLGCGVTTGFGAALKTANVQKGDTVAVFGCGTVGLSVIQGAKLRGASKIIAIDINNKKKQYCSQFGATDFVNPKEDLAKDQTIVEKLIEMTDGGLDFTFDCTGNTKIMRDALEACHKGWGQSIIIGVAAAGEEISTRPFQLVTGRVWKGSAFGGIKGRSEMGGLIKDYQKGALKVEEFITHRRPFKEINQAFEDLHNGDCLRTVLKSDEIK

SEQ ID NO: 145 - protein, 342 aa, Saccharomyces cerevisiae
MVLVKQVRLGNSGLKISPIVIGCMSYGSKKWADWVIEDKTQIFKIMKHCYDKGLRTFDTADFYSNGLSERIIKEFLEYYSIKRETVVIMTKIYFPVDETLDLHHNFTLNEFEELDLSNQRGLSRKHIIAGVENSVKRLGTYIDLLQIHRLDHETPMKEIMKALNDVVEAGHVRYIGASSMLATEFAELQFTADKYGWFQFISSQSYYNLLYREDERELIPFAKRHNIGLLPWSPNARGMLTRPLNQSTDRIKSDPTFKSLHLDNLEEEQKEIINRVEKVSKDKKVSMAMLSIAWVLHKGCHPIVGLNTTARVDEAIAALQVTLTEEEIKYLEEPYKPQRQRC

SEQ ID NO: 146 - protein, 359 aa, Saccharomyces cerevisiae
MGVEQILKRKTGVIVGEDVHNLFTYAKEHKFAIPAINVTSSSTAVAALEAARDSKSPIILQTSNGGAAYFAGKGISNEGQNASIKGAIAAAHYIRSIAPAYGIPVVLHSDHCAKKLLPWFDGMLEADEAYFKEHGEPLFSSHMLDLSEETDEENISTCVKYFKRMAAMDQWLEMEIGITGGEEDGVNNENADKEDLYTKPEQVYNVYKALHPISPNFSIAAAFGNCHGLYAGDIALRPEILAEHQKYTREQVGCKEEKPLFLVFHGGSGSTVQEFHTGIDNGVVKVNLDTDCQYAYLTGIRDYVLNKKDYIMSPVGNPEGPEKPNKKFFDPRVWVREGEKTMGAKITKSLETFRTTNTL

SEQ ID NO: 147 - protein, 248 aa, Saccharomyces cerevisiae
MARTFFVGGNFKLNGSKQSIKEIVERLNTASIPENVEVVICPPATYLDYSVSLVKKPQVTVGAQNAYLKASGAFTGENSVDQIKDVGAKWVILGHSERRSYFHEDDKFIADKTKFALGQGVGVILCIGETLEEKKAGKTLDVVERQLNAVLEEVKDWTNVVVAYEPVWAIGTGLAATPEDAQDIHASIRKFLASKLGDKAASELRILYGGSANGSNAVTFKDKADVDGFLVGGASLKPEFVDIINSRN

SEQ ID NO: 148 - protein, 376 aa, Saccharomyces cerevisiae
MSKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYDYQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQNGSYATRAYGQKK

SEQ ID NO: 149 - protein, 327 aa, Saccharomyces cerevisiae
MSSLVTLNNGLKMPLVGLGCWKIDKKVCANQIYEAIKLGYRLFDGACDYGNEKEVGEGIRKAISEGLVSRKDIFVVSKLWNNFHHPDHVKLALKKTLSDMGLDYLDLYYIHFPIAFKYVPFEEKYPPGFYTGADDEKKGHITEAHVPIIDTYRALEECVDEGLIKSIGVSNFQGSLIQDLLRGCRIKPVALQIEHHPYLTQEHLVEFCKLHDIQVVAYSSFGPQSFIEMDLQLAKTTPTLFENDVIKKVSQNHPGSTTSQVLLRWATQRGIAVIPKSSKKERLLGNLEIEKKFTLTEQELKDISALNANIRFNDPWTWLDGKFPTFA

SEQ ID NO: 150 - protein, 350 aa, Saccharomyces cerevisiae
MSKKPIVLKLGKDAFGDQAWGELEKIADVITIPESTTREQFLREVKDPQNKLSQVQVITRTARSVKNTGRFDEELALALPSSVVAVCHTGAGYDQIDVEPFKKRHIQVANVPDLVSNATADTHVFLLLGALRNFGIGNRRLIEGNWPEAGPACGSPFGYDPEGKTVGILGLGRIGRCILERLKPFGFENFIYHNRHQLPSEEEHGCEYVGFEEFLKRSDIVSVNVPLNHNTHHLINAETIEKMKDGVVIVNTARGAVIDEQAMTDALRSGKIRSAGLDVFEYEPKISKELLSMSQVLGLPHMGTHSVETRKKMEELVVENAKNVILTGKVLTIVPELQNEDWPNESKPLV

SEQ ID NO: 151 - protein, 396 aa, Saccharomyces cerevisiae
MITSIDIADVTYSAKPRILVPYKTQWEVASHLPEYRKLAERVEFYKYEMSTKDDFVKFLETHRINGFWLTEEFFTVLGNPSSYIEFFPASLKVILVPWVGCDFIDGKLLRSKGITLCNIGPHAADHVTELAIFLAISCFRMTSFWEYCFKYVENGNVEQCKKYISSDSYEIVTDSYHGQEMKFPSRTDKCKPNKDRKVVHLAEKYTVGGKKMESPMNKKVLILGFGSIGQNIGSNLHKVFNMSIEYYKRTGPVQKSLLDYNAKYHSDLDDPNTWKNADLIILALPSTASTNNIINRKSLAWCKDGVRIVNVGRGTCIDEDVLLDALESGKVASCGLDVFKNEETRVKQELLRRWDVTALPHIGSTVADMVIKQTLITLENVQDIFVEGGDGKYVLN

SEQ ID NO: 152 - protein, 312 aa, Saccharomyces cerevisiae
MPATLHDSTKILSLNTGAQIPQIGLGTWQSKENDAYKAVLTALKDGYRHIDTAAIYRNEDQVGQAIKDSGVPREEIFVTTKLWCTQHHEPEVALDQSLKRLGLDYVDLYLMHWPARLDPAYIKNEDILSVPTKKDGSRAVDITNWNFIKTWELMQELPKTGKTKAVGVSNFSINNLKDLLASQGNKLTPAANQVEIHPLLPQDELINFCKSKGIVVEAYSPLGSTDAPLLKEPVILEIAKKNNVQPGHVVISWHVQRGYVVLPKSVNPDRIKTNRKIFTLSTEDFEAINNISKEKGEKRVVHPNWSPFEVFK

SEQ ID NO: 153 - protein, 505 aa, Saccharomyces cerevisiae
MPTLYTDIEIPQLKISLKQPLGLFINNEFCPSSDGKTIETVNPATGEPITSFQAANEKDVDKAVKAARAAFDNVWSKTSSEQRGIYLSNLLKLIEEEQDTLAALETLDAGKPYSNAKGDLAQILQLTRYFAGSADKFDKGATIPLTFNKFAYTLKVPFGVVAQIVPWNYPLAMACWKLQGALAAGNTVIIKPAENTSLSLLYFATLIKKAGFPPGVVNIVPGYGSLVGQALASHMDIDKISFTGSTKVGGFVLEASGQSNLKDVTLECGGKSPALVFEDADLDKAIDWIAAGIFYNSGQNCTANSRVYVQSSIYDKFVEKFKETAKKEWDVAGKFDPFDEKCIVGPVISSTQYDRIKSYIERGKREEKLDMFQTSEFPIGGAKGYFIPPTIFTDVPQTSKLLQDEIFGPVVVVSKFTNYDDALKLANDTCYGLASAVFTKDVKKAHMFARDIKAGTVWINSSNDEDVTVPFGGFKMSGIGRELGQSGVDTYLQTKAVHINLSLDN

SEQ ID NO: 154 - protein, 506 aa, Saccharomyces cerevisiae
MPTLYTDIEIPQLKISLKQPLGLFINNEFCPSSDGKTIETVNPATGEPITSFQAANEKDVDKAVKAARAAFDNVWSKTSSEQRGIYLSNLLKLIEEEQDTLAALETLDAGKPFHSNAKQDLAQIIELTRYYAGAVDKFNMGETIPLTFNKFAYTLKVPFGVVAQIVPWNYPLAMACRKMQGALAAGNTVIIKPAENTSLSLLYFATLIKKAGFPPGVVNVIPGYGSVVGKALGTHMDIDKISFTGSTKVGGSVLEASGQSNLKDITLECGGKSPALVFEDADLDKAIEWVANGIFFNSGQICTANSRVYVQSSIYDKFVEKFKETAKKEWDVAGKFDPFDEKCIVGPVISSTQYDRIKSYIERGKKEEKLDMFQTSEFPIGGAKGYFIPPTIFTDVPETSKLLRDEIFGPVVVVSKFTNYDDALKLANDTCYGLASAVFTKDVKKAHMFARDIKAGTVWINQTNQEEAKVPFGGFKMSGIGRESGDTGVDNYLQIKSVHVDLSLDK

SEQ ID NO: 155 - protein, 519 aa, Saccharomyces cerevisiae
MFSRSTLCLKTSASSIGRLQLRYFSHLPMTVPIKLPNGLEYEQPTGLFINNKFVPSKQNKTFEVINPSTEEEICHIYEGREDDVEEAVQAADRAFSNGSWNGIDPIDRGKALYRLAELIEQDKDVIASIETLDNGKAISSSRGDVDLVINYLKSSAGFADKIDGRMIDTGRTHFSYTKRQPLGVCGQIIPWNFPLLMWAWKIAPALVTGNTVVLKTAESTPLSALYVSKYIPQAGIPPGVINIVSGFGKIVGEAITNHPKIKKVAFTGSTATGRHIYQSAAAGLKKVTLELGGKSPNIVFADAELKKAVQNIILGIYYNSGEVCCAGSRVYVEESIYDKFIEEFKAASESIKVGDPFDESTFQGAQTSQMQLNKILKYVDIGKNEGATLITGGERLGSKGYFIKPTVFGDVKEDMRIVKEEIFGPVVTVTKFKSADEVINMANDSEYGLAAGIHTSNINTALKVADRVNAGTVWINTYNDFHHAVPFGGFNASGLGREMSVDALQNYLQVKAVRAKLDE

SEQ ID NO: 156 - protein, 520 aa, Saccharomyces cerevisiae
MLSRTRAAAPNSRIFTRSLLRLYSQAPLRVPITLPNGFTYEQPTGLFINGEFVASKQKKTFDVINPSNEEKITTVYKAMEDDVDEAVAAAKKAFETKWSIVEPEVRAKALFNLADLVEKHQETLAAIESMDNGKSLFCARGDVALVSKYLRSCGGWADKIYGNVIDTGKNHFTYSIKEPLGVCGQIIPWNFPLLMWSWKIGPALATGNTVVLKPAETTPLSALFASQLCQEAGIPAGVVNILPGSGRVVGERLSAHPDVKKIAFTGSTATGRHIMKVAADTVKKVTLELGGKSPNIVFADADLDKAVKNIAFGIFYNSGEVCCAGSRIYIQDTVYEEVLQKLKDYTESLKVGDPFDEEVFQGAQTSDKQLHKILDYVDVAKSEGARLVTGGARHGSKGYFVKPTVFADVKGDMRIVKEEVFGPIVTVSKFSTVDEVIAMANDSQYGLAAGIHTNDINKAVDVSKRVKAGTVWINTYNNFHQNVPFGGFGQSGIGREMGEAALSNYTQTKSVRIAIDKPIR

SEQ ID NO: 157 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 158 - protein, 532 aa, Saccharomyces cerevisiae
MSNDGSKILNYTPVSKIDEIVEISRNFFFEKQLKLSHENNPRKKDLEFRQLQLKKLYYAVKDHEEELIDAMYKDFHRNKIESVLNETTKLMNDILHLIEILPKLIKPRRVSDSSPPFMFGKTIVEKISRGSVLIIAPFNFPLLLAFAPLAAALAAGNTIVLKPSELTPHTAVVMENLLTTAGFPDGLIQVVQGAIDETTRLLDCGKFDLIFYTGSPRVGSIVAEKAAKSLTPCVLELGGKSPTFITENFKASNIKIALKRIFFGAFGNSGQICVSPDYLLVHKSIYPKVIKECESVLNEFYPSFDEQTDFTRMIHEPAYKKAVASINSTNGSKIVPSKISINSDTEDLCLVPPTIVYNIGWDDPLMKQENFAPVLPIIEYEDLDETINKIIEEHDTPLVQYIFSDSQTEINRILTRLRSGDCVVGDTVIHVGITDAPFGGIGTSGYGNYGGYYGFNTFSHERTIFKQPYWNDFTLFMRYPPNSAQKEKLVRFAMERKPWFDRNGNNKWGLRQYFSLSAAVILISTIYAHCSS

SEQ ID NO: 159 - protein, 500 aa, Saccharomyces cerevisiae
MSFDDLHKATERAVIQAVDQICDDFEVTPEKLDELTAYFIEQMEKGLAPPKEGHTLASDKGLPMIPAFVTGSPNGTERGVLLAADLGGTNFRICSVNLHGDHTFSMEQMKSKIPDDLLDDENVTSDDLFGFLARRTLAFMKKYHPDELAKGKDAKPMKLGFTFSYPVDQTSLNSGTLIRWTKGFRIADTVGKDVVQLYQEQLSAQGMPMIKVVALTNDTVGTYLSHCYTSDNTDSMTSGEISEPVIGCIFGTGTNGCYMEEINKITKLPQELRDKLIKEGKTHMIINVEWGSFDNELKHLPTTKYDVVIDQKLSTNPGFHLFEKRVSGMFLGEVLRNILVDLHSQGLLLQQYRSKEQLPRHLTTPFQLSSEVLSHIEIDDSTGLRETELSLLQSLRLPTTPTERVQIQKLVRAISRRSAYLAAVPLAAILIKTNALNKRYHGEVEIGCDGSVVEYYPGFRSMLRHALALSPLGAEGERKVHLKIAKDGSGVGAALCALVA

SEQ ID NO: 160 - protein, 554 aa, Saccharomyces cerevisiae
MSNNSFTNFKLATELPAWSKLQKIYESQGKTLSVKQEFQKDAKRFEKLNKTFTNYDGSKILFDYSKNLVNDEIIAALIELAKEANVTGLRDAMFKGEHINSTEDRAVYHVALRNRANKPMYVDGVNVAPEVDSVLKHMKEFSEQVRSGEWKGYTGKKITDVVNIGIGGSDLGPVMVTEALKHYAGVLDVHFVSNIDGTHIAETLKVVDPETTLFLIASKTFTTAETITNANTAKNWFLSKTGNDPSHIAKHFAALSTNETEVAKFGIDTKNMFGFESWVGGRYSVWSAIGLSVALYIGYDNFEAFLKGAEAVDNHFTQTPLEDNIPLLGGLLSVWYNNFFGAQTHLVAPFDQYLHRFPAYLQQLSMESNGKSVTRGNVFTDYSTGSILFGEPATNAQHSFFQLVHQGTKLIPSDFILAAQSHNPIENKLHQKMLASNFFAQAEALMVGKDEEQVKAEGATGGLVPHKVFSGNRPTTSILAQKITPATLGALIAYYEHVTFTEGAIWNINSFDQWGVELGKVLAKVIGKELDNSSTISTHDASTNGLINQFKEWM

SEQ ID NO: 161 - protein, 987 aa, Saccharomyces cerevisiae
MQSQDSCYGVAFRSIITNDEALFKKTIHFYHTLGFATVKDFNKFKHGENSLLSSGTSQDSLREVWLESFKLSEVDASGFRIPQQEATNKAQSQGALLKIRLVMSAPIDETFDTNETATITYFSTDLNKIVEKFPKQAEKLSDTLVFLKDPMGNNITFSGLANATDSAPTSKDAFLEATSEDEIISRASSDASDLLRQTLGSSQKKKKIAVMTSGGDSPGMNAAVRAVVRTGIHFGCDVFAVYEGYEGLLRGGKYLKKMAWEDVRGWLSEGGTLIGTARSMEFRKREGRRQAAGNLISQGIDALVVCGGDGSLTGADLFRHEWPSLVDELVAEGRFTKEEVAPYKNLSIVGLVGSIDNDMSGTDSTIGAYSALERICEMVDYIDATAKSHSRAFVVEVMGRHCGWLALMAGIATGADYIFIPERAVPHGKWQDELKEVCQRHRSKGRRNNTIIVAEGALDDQLNPVTANDVKDALIELGLDTKVTILGHVQRGGTAVAHDRWLATLQGVDAVKAVLEFTPETPSPLIGILENKIIRMPLVESVKLTKSVATAIENKDFDKAISLRDTEFIELYENFLSTTVKDDGSELLPVSDRLNIGIVHVGAPSAALNAATRAATLYCLSHGHKPYAIMNGFSGLIQTGEVKELSWIDVENWHNLGGSEIGTNRSVASEDLGTIAYYFQKNKLDGLIILGGFEGFRSLKQLRDGRTQHPIFNIPMCLIPATVSNNVPGTEYSLGVDTCLNALVNYTDDIKQSASATRRRVFVCEVQGGHSGYIASFTGLITGAVSVYTPEKKIDLASIREDITLLKENFRHDKGENRNGKLLVRNEQASSVYSTQLLADIISEASKGKFGVRTAIPGHVQQGGVPSSKDRVTASRFAVKCIKFIEQWNKKNEASPNTDAKVLRFKFDTHGEKVPTVEHEDDSAAVICVNGSHVSFKPIANLWENETNVELRKGFEVHWAEYNKIGDILSGRLKLRAEVAALAAENK

SEQ ID NO: 162 - protein, 959 aa, Saccharomyces cerevisiae
MTVTTPFVNGTSYCTVTAYSVQSYKAAIDFYTKFLSLENRSSPDENSTLLSNDSISLKILLRPDEKINKNVEAHLKELNSITKTQDWRSHATQSLVFNTSDILAVKDTLNAMNAPLQGYPTELFPMQLYTLDPLGNVVGVTSTKNAVSTKPTPPPAPEASAESGLSSKVHSYTDLAYRMKTTDTYPSLPKPLNRPQKAIAVMTSGGDAPGMNSNVRAIVRSAIFKGCRAFVVMEGYEGLVRGGPEYIKEFHWEDVRGWSAEGGTNIGTARCMEFKKREGRLLGAQHLIEAGVDALIVCGGDGSLTGADLFRSEWPSLIEELLKTNRISNEQYERMKHLNICGTVGSIDNDMSTTDATIGAYSALDRICKAIDYVEATANSHSRAFVVEVMGRNCGWLALLAGIATSADYIFIPEKPATSSEWQDQMCDIVSKHRSRGKRTTIVVVAEGAIAADLTPISPSDVHKVLVDRLGLDTRITTLGHVQRGGTAVAYDRILATLQGLEAVNAVLESTPDTPSPLIAVNENKIVRKPLMESVKLTKAVAEAIQAKDFKRAMSLRDTEFIEHLNNFMAINSADHNEPKLPKDKRLKIAIVNVGAPAGGINSAVYSMATYCMSQGHRPYAIYNGWSGLARHESVRSLNWKDMLGWQSRGGSEIGTNRVTPEEADLGMIAYYFQKYEFDGLIIVGGFEAFESLHQLERARESYPAFRIPMVLIPATLSNNVPGTEYSLGSDTALNALMEYCDVVKQSASSTRGRAFVVDCQGGNSGYLATYASLAVGAQVSYVPEEGISLEQLSEDIEYLAQSFEKAEGRGRFGKLILKSTNASKALSATKLAEVITAEADGRFDAKPAYPGHVQQGGLPSPIDRTRATRMAIKAVGFIKDNQAAIAEARAAEENFNADDKTISDTAAVVGVKGSHVVYNSIRQLYDYETEVSMRMPKVIHWQATRLIADHLVGRKRVD

SEQ ID NO: 163 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 164 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 165 - protein, 563 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVIRLIELKLPVFDAPESLIKQAQLTAATNAKQ

SEQ ID NO: 166 - protein, 440 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPLFEAVYQIVYNNVRMEDLPEMIEELDIDDE

SEQ ID NO: 167 - protein, 250 aa, Saccharomyces cerevisiae
MPLTTKPLSLKINAALFDVDGTIIISQPAIAAFWRDFGKDKPYFDAEHVIHISHGWRTYDAIAKFAPDFADEEYVNKLEGEIPEKYGEHSIEVPGAVKLCNALNALPKEKWAVATSGTRDMAKKWFDILKIKRPEYFITANDVKQGKPHPEPYLKGRNGLGFPINEQDPSKSKVVVFEDAPAGIAAGKAAGCKIVGIATTFDLDFLKEKGCDIIVKNHESIRVGEYNAETDEVELIFDDYLYAKDDLLKW

(19) United States
(12) Patent Application Publication
(10) Pub. No.: US 2013/0052646 A1
(43) Pub. Date: Feb. 28, 2013

(54) POSITIVE AND NEGATIVE SELECTABLE MARKERS FOR USE IN THERMOPHILIC ORGANISMS

(71) Applicants: Shital Tripathi, Emeryville, CA (US); Aaron Argyros, White River Junction, VT (US); Trisha Barrett, Bradford, VT (US); Nicky Caiazza, Rancho Santa Fe, CA (US); Bethany B. Miller, Orford, NH (US); Arthur J. Shaw, IV, Grantham, NH (US)

(72) Inventors: Shital Tripathi, Emeryville, CA (US); Aaron Argyros, White River Junction, VT (US); Trisha Barrett, Bradford, VT (US); Nicky Caiazza, Rancho Santa Fe, CA (US); Bethany B. Miller, Orford, NH (US); Arthur J. Shaw, IV, Grantham, NH (US)

(73) Assignee: Mascoma Corporation

(21) Appl. No.: 13/393,093

(22) PCT Filed: Aug. 10, 2010
(86) PCT No.: PCT/US10/45019

§ 371 Date: Oct. 11, 2012

(30) Foreign Application Priority Data
Aug. 10, 2009 (US) 61232648

(51) Int. Cl. C12N 15/74; C12Q 1/68; C12N 1/21

(52) U.S. Cl. 435/6.12; 435/320.1; 435/252.3; 435/471

(57) ABSTRACT

The present invention relates to the field of molecular biology and genetic tool development in thermophilic bacteria. In particular, it relates to the use of positive and/or negative selection markers that can be used to efficiently select modified strains of interest. By providing such capabilities, the disclosed invention facilitates the recycling of genetic markers in thermophilic bacterial host cells. The present invention also allows the creation of unmarked strains. The genetic tools disclosed in the present invention are prerequisites for making targeted higher order mutations in a single thermophilic strain background.

### REFERENCE TO SEQUENCE LISTING SUBMITTED ELECTRONICALLY

The content of the electronically submitted sequence listing in ASCII text file (Sequence Listing.ST25.txt; Size: 196,608 bytes; and Date of Creation: Aug. 10, 2009) filed with the application is incorporated herein by reference in its entirety.

### BACKGROUND OF THE INVENTION

1. Field of the Invention

The present invention relates to the field of molecular biology and genetic tool development in thermophilic bacteria. In particular, it relates to the use of positive and/or negative selection markers that can be used to efficiently select modified strains of interest. By providing such capabilities, the disclosed invention facilitates the recycling of genetic markers in thermophilic bacterial host cells. The present invention also allows the creation of unmarked strains. The genetic tools disclosed in the present invention are prerequisites for making targeted higher order mutations in a single thermophilic strain background.

2. Background Art

Thermophilic microorganisms, which can grow at temperatures of 45° C. and above, are useful for a variety of industrial processes. For example, thermophilic microorganisms can be used as biocatalysts in reactions at higher operating temperatures than can be achieved with mesophilic microorganisms. Thermophilic organisms are particularly useful in biologically mediated processes for energy conversion, such as the production of ethanol from plant biomass, because higher operating temperatures allow more convenient and efficient removal of ethanol in vaporized form from the fermentation medium. Thermophilic organisms can also be used for the generation of alternative products including lactate or acrylate.

The ability to metabolically engineer thermophilic microorganisms to improve various properties (e.g., ethanol production, breakdown of lignocellulosic materials), would allow the benefit of higher operating temperatures to be combined with the benefits of using industrially important enzymes from a variety of sources in order to improve efficiency and lower the cost of production of various industrial processes, such as energy conversion and alternative fuel production.

Thermophilic organisms such as C. thermocellum and T. saccharolyticum are rapidly becoming organisms of choice for their potential to produce ethanol from cellulosic material. The genetic engineering of such thermophiles is necessary for the development of an efficient consolidated bioprocessing (CBP) system in the production of cellulosic ethanol and alternative products such as lactate or acrylate. A critical step towards genetic engineering of thermophiles is the development of specialized genetic tools.

Positive and negative selection markers greatly facilitate the ability to recycle genetic markers and make unmarked gene deletions, both of which are prerequisites for making targeted higher order mutations in a single strain background. The latter is required to make C. thermocellum a high yielding ethanologen. To date, higher order mutations have not been possible to achieve in C. thermocellum due to the limited number of genetic markers available in this system.

Positive and negative selections are commonly used genetic tools and have been applied to many classes of microbes. Many different types of positive and negative selections exist. However, little of this technology has been transferred to the anaerobic thermophiles. This is especially true of the cellulolytic clostridia, such as C. thermocellum, that fall into this class. In terms of negative selectable markers, not much information is known with respect to their use in anaerobic thermophilic organisms.

The choice of selection markers for use in anaerobic thermophiles is also complicated by the fact that prior selection systems typically are not adaptable to thermophilic systems. In addition, many of the pathways in which selectable markers function have not been clearly elucidated in thermophiles. Furthermore, whether traditional selection schemes are operable under temperature and pH conditions required for the growth of thermophiles is also unpredictable. Thus, it is unclear whether thermophilic organisms harbor homologs of well-known marker genes and whether they would function as expected. Attempts to utilize selection markers commonly used in other systems have resulted in inadequate growth of strains, the inability to efficiently select for the presence or absence of the marker, and a failure of selection due to a lack of information regarding the potential of such a marker to function in the thermophilic host.

Applicants have recognized the potential of certain selection markers to be applied towards the genetic engineering of thermophiles. These markers include the URA3 bacterial homolog, pyrF, as well as thymidine kinase (tdk) and hypoxanthine phosophoribosyl transferase (hpt). The pyrF gene has been successfully utilized in various systems, but has not been extensively applied to thermophilic organisms.

The tdk gene has been used as a negative selectable marker to make targeted gene deletions in other systems, such as the gram-negative bacterium Acinetobacter sp. and the gram-positive bacterium Streptococcus gordonii. See Metzgar et al., NAR 32: 5780-5790 (2004) and Franke et al., Antimicrobial Agents and Chemotherapy 44: 787-789 (200). However, no negative selection tools have been shown to be successful for use in C. thermocellum.

In addition, it is well known that the hypoxanthine phosophoribosyl transferase (hpt) gene is sensitive to the anti-metabolites 8-azahypoxanthine, 6-mercaptopurine, 8-azaguanine, aza-2,6-diaminopurine, and 6-thioguanine. There are multiple reports of using these anti-metabolites to delete the hpt gene and turn it into a negative selectable marker. However, there are no reports utilizing an artificial operon expressing an antibiotic resistance gene and hpt or tdk. In addition, there are no published reports using hpt as a marker in thermophilic or cellulolytic organisms. Excluding mammalian systems, there are very few reports detailing the use of hpt as a positive selectable marker. Such reports include those that describe the use of hpt in non-thermophilic organisms such as Toxiplasma gondii, Methanococcus maripaludis, Methanosarcina acetivorans and vaccinia virus. See Donald and Roos, Mol. Biochem. Parasitol. 91:295-305 (1998); Donald et al., J. Biol. Chem. 271:14010-9 (1996); Moore and Leigh, J. Bacteriol. 187:972-9 (2005); Prtichett et al., Appl. Environ. Microbiol. 70:1425-33 (2004); Isaacs et al., Virology 178:626-30 (1990).

The use of tdk and hpt as potential positive and negative selectable markers in mammalian cell culture was first reported in 1962 with the development of the HAT medium selection technique (http://en.wikipedia.org/wiki/HAT_medium). The HAT selection technique has been refined and modified over the past five decades utilizing both hpt and tdk in various ways. The use of either tdk or hpt as selectable markers in thermophiles or cellulolytic organisms has not, however, been reported for thermophilic organisms.

The present invention provides genetic tools for use in anaerobic thermophiles, including vector constructs for positive and/or negative selection and methods of utilizing such constructs for the recycling of genetic markers and for the creation of unmarked strains. In particular, the present invention provides for vector constructs containing a combination of markers, including pyrF, tdk and/or hpt, and optionally one or more antibiotic resistance markers. The present invention demonstrates the application of these selectable markers, and the use of both positive and negative selection capabilities, for the genetic engineering of thermophilic organisms.

### BRIEF SUMMARY OF THE INVENTION

The present invention provides for a vector for use in an anaerobic thermophilic host comprising: (a) one or more selectable marker sequences, wherein each selectable marker sequence comprises a nucleic acid sequence encoding for a positive and/or negative selectable marker; and (b) a thermophilic host sequence; wherein said thermophilic host sequence comprises a nucleic acid sequence that is endogenous to said thermophilic host.

In additional embodiments, the selectable markers are selected from the group consisting of thymidine kinase (tdk), hypoxanthine phosphoribosyltransferase (hpt) and orotidine-5°-phosphate decarboxylase (pyrF), an antibiotic resistance marker or a combination thereof. In certain embodiments, the selectable markers are derived from an anaerobic thermophilic organism, including a heterologous anaerobic thermophilic organism. In a further aspect, the invention provides that the tdk is from Thermoanaerobacterium saccharolyticum. In other aspects, the invention provides that the pyrF or hpt is from Clostridium thermocellum or Thermoanaerobacterium saccharolyticum.

In further embodiments, the vector comprises at least one positive selectable marker sequence, at least one negative selectable marker sequence, at least two selectable marker sequences or at least one positive selectable marker sequence and at least one negative selectable marker sequence. In particular embodiments, the selectable marker sequence encodes for a selectable marker that provides for both positive and negative selection. In other embodiments, the selectable marker sequence encodes for a selectable marker that provides for positive or negative selection.

In additional embodiments, the invention provides that the anaerobic thermophilic host sequence of the vector comprises nucleic acid sequences of regions flanking an endogenous target gene, an endogenous replicon, an endogenous origin of replication, or an endogenous regulatory sequence. In certain embodiments, the anaerobic thermophilic host is a xylanolytic and/or cellulolytic thermophilic organism. In certain other embodiments, the thermophilic host is Clostridium thermocellum or Thermoanaerobacterium saccharolyticum.

The invention further provides for a thermophilic host cell comprising a vector according to the invention. In particular embodiments, the endogenous hpt gene of the thermophilic host cell has been deleted (Δhpt). In certain other embodiments, the thermophilic host is not auxotrophic.

The invention also provides for a method for producing a transformed thermophilic host cell, said method comprising the following steps: (a) transforming said thermophilic host cell with the vector according to the invention; and (b) selecting said host cell for the presence of said vector within the host cell.

The invention also provides for a method of making an unmarked thermophilic host cell, said method comprising the following steps: (a) transforming said thermophilic host cell with the vector according to the invention; (b) selecting said host cell for the presence of said vector within the host cell; (c) culturing said host cell for a length of time and under conditions whereby the vector replicates; and (d) selecting said host cell for the absence of said vector within the host cell.

The invention further provides for a method of making one or more targeted gene deletions in a thermophilic host cell, said method comprising the following steps: (a) transforming said thermophilic host cell with the vector according to the invention, wherein said vector comprises a thermophilic host sequence flanking an endogenous target gene; (b) selecting said host cell for the presence of said vector within the host cell; (c) culturing said host cell for a length of time and under conditions whereby homologous recombination occurs between the vector and the host cell genome; and (d) determining whether said target gene has been deleted; and, optionally, (e) repeating steps (a)-(d) for deletion of a different target gene. In additional embodiments, the target gene encodes for pta or ldh.

The invention also provides for a method for recycling genetic markers in a thermophilic host cell, said method comprising the following steps: (a) transforming said thermophilic host cell with the vector according to the invention; (b) selecting said host cell for the presence of said vector within the host cell; (c) culturing said host cell for a length of time and under conditions whereby the vector replicates; and (d) selecting said host cell for the absence of said vector within the host cell; and, optionally, (e) repeating steps (a)-(d).

The invention additionally provides for a thermophilic host cell produced by a method according to the invention.

### BRIEF DESCRIPTION OF THE DRAWINGS/FIGURES

FIG. 1. Diagram of plasmid pMU482 and schematic showing double cross over homologous recombination to make an in-frame deletion of the pyrF gene.

FIGS. 2A and B. Diagrams of control plasmid pMU102, and replicative knockout vector pMU482 used in the construction of a C. thermocellum pyrF mutant.

FIG. 3. Schematic showing strategy for the construction of a C. thermocellum pyrF (ura3) mutant using the replicative knockout vector pMU482.

FIG. 4. Photographic images of plates containing 5-fluoro-orotic acid (5-FOA) showing colony growth of cells transformed with control plasmid pMU102 or pyrF knockout plasmid pMU482. For each set of plates shown, the top left plate corresponds to undiluted culture, the top right corresponds to a 10⁻¹ dilution, the bottom left corresponds to a 10⁻² dilution and the bottom right corresponds to a 10⁻³ dilution.

FIG. 5. Photographic image displaying results of PCR analysis. Colonies resistant to 5-FOA were analyzed to determine whether the pyrF gene had been deleted. As indicated by the arrow on the right indicating where the resulting deletion fragment should migrate, several of the colonies (lanes 2, 4, 7-9 and 11) harbor the pyrF deletion.

FIG. 6. Photographic image of a gel depicting whether the loss of the knockout plasmid occurred. Colony #3 shows a loss of the pyrF knockout plasmid as indicated by the lack of product representing the pMU482 plasmid.

FIG. 7. Photographic image of plates depicting results of a positive selection experiment for the C. thermocellum pyrF knockout strain.

FIG. 8. Bar graph depicting results of a negative selection experiment for the C. thermocellum pyrF knockout strain.

FIG. 9. Schematic showing recombination events for plasmid pMU1162 in experiments using pyrF as a negative selectable marker.

FIG. 10. Photographic image of a gel showing the results of PCR screening using pyrF as a negative selectable marker. Colonies in which the pyrF gene was replaced with cat are indicated by boxes around the band of interest.

FIG. 11. Schematic showing recombination events for plasmid pMU1663 in experiments using pyrF as a positive selectable marker.

FIG. 12. Photographic image of a gel showing the results of PCR screening using pyrF as a positive selectable marker.

FIG. 13. A schematic of a metabolic pathway in C. thermocellum. The enzyme activity encoded by the tdk gene is indicted by EC#2.7.1.21. The white boxes indicate enzyme activities for which bioinformatic studies did not yield a corresponding encoded open reading frame (orf) in the genome. The activity for tdk (2.7.1.21) is such an example.

FIG. 14. A schematic of a metabolic pathway in T. saccharolyticum. The enzyme activity encoded by the tdk gene is indicted by EC#2.7.1.21. Green boxes indicate enzyme activities for which bioinformatic studies yield a corresponding encoded open reading frame (orf) in the genome. The activity for tdk (2.7.1.21) is such an example.

FIG. 15. Schematic showing the mechanism by which FUDR is a toxic antimetabolite in the presence of the enzyme activity encoded by the tdk gene.

FIG. 16. Diagram of plasmid pMU1452.

FIG. 17. Diagram of plating strategy used to generate the C. thermocellum pyrF::tdk strain and PCR screening of results of colonies representing this mutation.

FIG. 18. Photographic image of plates in negative selection experiments of the C. thermocellum pyrF::tdk strain.

FIG. 19. Schematic showing the strategy for plasmid curing of pMU1452.

FIG. 20. Photographic image of PCR plates and PCR results in plasmid curing experiment.

FIG. 21. Diagram depicting pathway for de novo purine synthesis when hpt is used as a negative selectable marker.

FIG. 22. Diagram depicting pathway for de novo purine synthesis when hpt is used as a positive selectable marker.

FIG. 23. Diagram of plasmid pMU1657.

FIG. 24. Gel image showing PCR products amplified using primers flanking the hpt locus. The letter G corresponds to the wild type genomic DNA used as template in the reaction. Lanes 1-6 are PCR's from colonies picked off plates containing 500 ug/ml 8-azahypoxanthine. Lane 7 (G) is C. thermocellum strain 1313 (wild-type) genomic DNA.

FIG. 25. Gel image showing PCR products amplified using primers specific for the hpt knockout plasmid. The letter P corresponds to the plasmid DNA (positive control). Lanes 1-6 represent individual colonies harboring the hpt deletion. The lack of product in these lanes indicates that the plasmid has been cured.

FIG. 26. Photographic image of plates showing colony growth. The plates show a 10⁻⁶ dilution of both the Δhpt strain and the Δhpt containing the complementing plasmid pMU1657. Both strains were plated with and without 500 ug/ml 8-AzaH.

FIG. 27. Diagram of plasmid pMU256.

FIG. 28. Photographic image of a gel showing results of PCR analysis indicating that the hpt gene was successfully deleted.

FIG. 29. Photographic images of plates showing that hpt can be utilized as a positive selectable marker. Both the C. thermocellum strain 1313 and the Δhpt strain were plated on several concentrations of mycophenolic acid. Colony growth represented the presence or absence of hpt.

FIG. 30. Diagram of plasmid pMU1589.

FIG. 31. Diagram of plasmid pMU1647.

FIG. 32. Diagram of plasmid pMU1687.

FIG. 33. Diagram of plasmid pMU1615.

FIG. 34. Diagram of plasmid pMU1616.

FIG. 35. Diagram of plasmid pMU1709.

FIG. 36. Diagram of plasmid pMU1676.

FIG. 37. Diagram of plasmid pMU1745.

FIG. 38. Gel image showing the results of PCR screening for integration of the cat-hpt operon and the duplicated upstream region.

FIG. 39. Gel image showing the results of PCR screening for deletion of Idh.

FIG. 40. Bar graph depicting results of batch fermentation experiments confirming the reduction of lactate production in strains harboring an ldh deletion.

FIG. 41. The scheme used to replace pta with cat expressed from the gapDH promoter in the C. thermocellum pyrF background is shown. MJ medium lacking uracil was used to select pyrF clones restored to uracil prototrophy as a result of being transformed with pMU1162 (FIG. 41A step 1). Single colonies representing transformants were propagated in liquid medium with Tm selection prior to plating on Tm plus 5-FOA (FIG. 41A Step 2). Panel B depicts a gel showing the expected fragment size in the deletion.

### DETAILED DESCRIPTION OF THE INVENTION

The present invention relates to, inter alia, the use of positive and/or negative selectable markers in thermophilic organisms. Applicants have constructed and characterized plasmids containing one or more of the selectable markers pyrF, tdk and hpt. Applicants' invention provides important tools for use in genetically engineering thermophilic microorganisms. In particular, Applicants' invention allows for recycling of genetic markers in thermophilic host cells and the creation of unmarked thermophilic strains.

### DEFINITIONS

A “plasmid” or “vector” refers to an extrachromosomal element often carrying one or more genes that are not part of the central metabolism of the cell, and is usually in the form of a circular double-stranded DNA molecule. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Preferably, the plasmids or vectors of the present invention are stable and self-replicating.

An “expression vector” is a vector that is capable of directing the expression of genes to which it is operably linked.

The term “thermophilic” refers to an organism that grows and thrives at a temperature of about 45° C. or higher.

The term “anaerobic” refers to an organism that grows and thrives under conditions of an absence of oxygen or under conditions of depleted nitrate, sulphate and/or oxygen.

A “selectable marker” is a gene, the expression of which creates a detectable phenotype and which facilitates detection of host cells that contain a plasmid having the selectable marker. Selectable markers include thymidine kinase (tdk), hypoxanthine phosphoribosyltransferase (hpt) and orotidine-5′-phosphate decarboxylase (pyrF). Additional non-limiting examples of selectable markers include drug resistance genes and nutritional markers. For example, the selectable marker can be a gene that confers resistance to an antibiotic selected from the group consisting of: ampicillin, kanamycin, erythromycin, chloramphenicol, gentamycin, kasugamycin, rifampicin, spectinomycin, D-Cycloserine, nalidixic acid, streptomycin, or tetracycline. Other non-limiting examples of selection markers include adenosine deaminase, aminoglycoside phosphotransferase, dihydrofolate reductase, hygromycin-B-phosphotransferase, thymidine kinase, and xanthine-guanine phosphoribosyltransferase. A single plasmid can comprise one or more selectable markers.

The term “FOA” or “5-FOA” refers to 5-fluoroorotic acid. Typically used in yeast molecular genetics to detect expression of the URA3 gene that encodes orotine-5′-monophosphate (OMP) dicarboxylase. Cells with an active URA3 gene (Ura+) (or the homolog pyrF) convert the 5-FOA to fluorodeoxyuridine, which is toxic to cells. Yeast strains carrying a mutation in the URA3 gene (or pyrF) grow in the presence of 5-FOA, if the media is supplemented with uracil.

The term “endogenous” as used herein means native to, or originating within, an organism or system, e.g., a component that is normally present, produced or synthesized within an organism or system.

The term “heterologous” as used herein refers to an element of a plasmid or cell that is derived from a source other than the endogenous source. Thus, for example, a heterologous sequence could be a sequence that is derived from a different gene or plasmid from the same host, from a different strain of host cell, or from an organism of a different taxonomic group (e.g., different kingdom, phylum, class, order, family genus, or species, or any subgroup within one of these classifications). The term “heterologous” is also used synonymously herein with the term “exogenous.”

The term “unmarked” as used herein means not having a particular identifying selectable marker. For example, an “unmarked strain” or “unmarked host cell” refers to a strain or host cell that does not contain a gene for one or more particular selectable markers, where the selectable markers can be present endogenously, present extrachromosomally (e.g., on a plasmid) or integrated into the genome. A “marked strain” or “marked host cell” means having a particular identifying selectable marker.

The term “recombination” refers to the physical exchange of DNA between two identical (homologous), or nearly identical, DNA molecules. Recombination is used for targeted gene deletion to modify the sequence of a gene.

A “targeted gene deletion” or “gene knockout” refers to a technique by which an organism is engineered such that a particular endogenous gene of interest has been made inoperative. A targeted gene deletion can be achieved by utilizing a vector construct that has been engineered to recombine with the endogenous target gene, which is accomplished by incorporating sequences from the target gene itself into the vector constnict flanking a foreign sequence. Recombination then occurs between the target gene sequences within the vector and the endogenous target gene sequences, resulting in the insertion of a foreign sequence to disrupt the gene. With its sequence interrupted, the altered gene in most cases will be translated into a nonfunctional protein, if it is translated at all. Because the desired type of DNA recombination is a rare event in the case of most cells and most constructs, the foreign sequence chosen for insertion usually includes a selectable marker. This enables selection of cells or organisms in which the targeted gene was successfully deleted. A rarer second recombination event can subsequently occur, resulting in the extraction of the selectable marker from the site of insertion. After several rounds of cell division, this extracted marker sequence can be lost, resulting in an unmarked strain harboring a targeted gene deletion.

“Flanking sequences” as used herein refers to short DNA sequences located on either side of a transcription unit or a genetic locus. Flanking sequences often do not code for a protein.

The term “recycling a genetic marker” refers to the use of a selectable marker for making, e.g., a targeted gene deletion, and then removing the marker gene to allow subsequent genetic manipulations with that same marker.

The term “auxotrophy” refers to the inability of an organism to synthesize a particular organic compound required for its growth. An auxotroph is an organism that displays this characteristic. A strain is said to be auxotrophic if it carries a mutation that renders it unable to synthesize an essential compound. For example a bacterial mutant in which a gene of the uracil synthesis pathway is inactivated is a uracil auxotroph. Such a strain is unable to synthesize uracil and will only be able to grow if uracil can be taken up from the environment.

The term “stable plasmid” refers to a plasmid that is capable of autonomous replication and which is maintained throughout at least one and preferably many successive generations of host cell division. A “thermostable plasmid” is a plasmid that is stable at the temperatures of a thermophilic host.

A “reporter gene” is a gene that produces a detectable product that is connected to a promoter of interest so that detection of the reporter gene product can be used to evaluate promoter function. A reporter gene may also be fused to a gene of interest (e.g., 3′ to the endogenous promoter of the gene of interest), such that the fused genes are expressed as a fusion protein that allow one to detect whether the gene of interest is expressed under a given set of conditions. Non-limiting examples of reporter genes include: β-galactosidase, β-glucuronidase, luciferase, chloramphenicol acetyltransferase (CAT), secreted alkaline phosphatase (SEAP), green fluorescent protein (GFP), red fluorescent protein (RFP), and catechol 2,3-oxygenase (xylE).

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA.

An “isolated nucleic acid molecule” or “isolated nucleic acid fragment” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

A “gene” refers to an assembly of nucleotides that encode a polypeptide, and includes cDNA and genomic DNA nucleic acids. “Gene” also refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

Suitable nucleic acid sequences or fragments thereof (including any of the isolated polynucleotides of the present invention) encode polypeptides that are at least about 70% to 75% identical to the amino acid sequences reported herein, preferably at least about 80%, 85%, or 90% identical to the amino acid sequences reported herein, and most preferably at least about 95%, 96%, 97%, 98%, 99%, or 100% identical to the amino acid sequences reported herein. Suitable nucleic acid fragments are preferably at least about 70%, 75%, or 80% identical to the nucleic acid sequences reported herein, preferably at least about 80%, 85%, or 90% identical to the nucleic acid sequences reported herein, and most preferably at least about 95%, 96%, 97%, 98%, 99%, or 100% identical to the nucleic acid sequences reported herein. Suitable nucleic acid fragments not only have the above identities/similarities but typically encode a polypeptide having at least 50 amino acids, preferably at least 100 amino acids, more preferably at least 150 amino acids, still more preferably at least 200 amino acids, and most preferably at least 250 amino acids.

A DNA “coding sequence” is a double-stranded DNA sequence which is transcribed and translated into a polypeptide in a cell in vitro or in vivo when placed under the control of appropriate regulatory sequences. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in a eukaryotic cell, a polyadenylation signal and transcription termination sequence will usually be located 3′ to the coding sequence.

“Open reading frame” is abbreviated ORF and means a length of nucleic acid sequence, either DNA, cDNA or RNA, that comprises a translation start signal or initiation codon, such as an ATG or AUG, and a termination codon and can be potentially translated into a polypeptide sequence.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression,” as used herein, refers to the transcription and stable accumulation of sense (mRNA) or antisense RNA derived from the nucleic acid fragment of the invention. Expression may also refer to translation of mRNA into a polypeptide.

The terms “restriction endonuclease” and “restriction enzyme” refer to an enzyme which binds and cuts at a specific nucleotide sequence within double stranded DNA.

The term “probe” refers to a single-stranded nucleic acid molecule that can base pair with a complementary single stranded target nucleic acid to foam a double-stranded molecule.

The term “complementary” is used to describe the relationship between nucleotide bases that are capable to hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing as well as those substantially similar nucleic acid sequences.

As used herein, the term “oligonucleotide” refers to a nucleic acid, generally of about 18 nucleotides, that is hybridizable to a genomic DNA molecule, a cDNA molecule, or an mRNA molecule. Oligonucleotides can be labeled, e.g., with ³²P-nucleotides or nucleotides to which a label, such as biotin, has been covalently conjugated. An oligonucleotide can be used as a probe to detect the presence of a nucleic acid according to the invention. Similarly, oligonucleotides (one or both of which may be labeled) can be used as PCR primers, either for cloning full length or a fragment of a nucleic acid of the invention, or to detect the presence of nucleic acids according to the invention. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. MOLECULAR CLONING: A LABORATORY MANUAL, Second Edition, Cold Spring Harbor Laboratory Press, Cold Spring Harbor (1989), particularly Chapter 11 and Table 11.1 therein (hereinafter “Maniatis”, entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. Another set of highly stringent conditions are defined by hybridization at 0.1×SSC, 0.1% SDS, 65° C. and washed with 2×SSC, 0.1% SDS followed by 0.1×SSC, 0.1% SDS.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see, e.g., Maniatis at 9.50-9.51). For hybridizations with shorter nucleic acids, i.e., oligonucleotides, the position of mismatches becomes more important, and the length of the oligonucleotide determines its specificity (see, e.g., Maniatis, at 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

The term “cellulase” refers to an enzyme involved in cellulose degradation. A cellulase can be an endoglucanase which cuts at random in the cellulose polysaccharide chain of amorphous cellulose generating oligosaccharides of varying lengths and consequently new chain ends, an exoglucanase which acts in a processive manner on the reducing or non-reducing ends of cellulose polysaccharide chains liberating either glucose (glucanohydrolases) or cellobiose (cellobiohydrolase) as major products or a β-glucosidase (β-glucoside glucohydrolases; EC 3.2.1.21) which hydrolyzes soluble cellodextrins and cellobiose to glucose units.

### Development of Genetic Tools in Thermophilic Organisms

As discussed above, a critical step in genetic engineering of thermophiles such as C. thermocellum is the development of specialized genetic tools. The present invention relates to such specialized genetic tools for use in anaerobic thermophiles, including vector constructs for positive and/or negative selection and methods of utilizing such constructs for the recycling of genetic markers and for the creation of unmarked strains.

In one aspect of the invention, a combination of selectable markers are utilized. A vector of the invention can include one or more, two or more, or three or more positive and/or negative selectable markers, including 1, 2, 3, 4, 5 or 6 positive and/or negative selectable markers. In certain embodiments, the selectable markers are selected from the group consisting of pyrF, tdk and hpt, chloramphenicol, thyamphenicol, neomycin, and kanamycin. Particular combinations of markers can include, for example: (1) tdk and hpt; (2) pyrF and hpt; (3) chloramphenicol, hpt, and tdk; (4) neomycin and tdk; (5) kanamycin, hpt and tdk; (6) chloramphenicol, tdk and hpt; (7) neomycin, tdk and hpt; or (8) kanamycin, tdk and hpt.

In certain embodiments, when more than two markers are utilized in a single vector, two of the markers can be present between flanking sequences homologous to an endogenous target gene, and one or more additional markers can be present on the vector outside of the flanking sequence. In addition, one or more of the markers can be present between the flanking sequences homologous to an endogenous target gene, and one or more additional markers can be present on the vector outside of the flanking sequence.

One aspect of the invention relates to the construction of a thermophilic strain harboring a targeted in frame clean gene deletion. As described above, a targeted gene deletion can be achieved by utilizing a replicating vector construct that has been engineered to recombine with a portion of the target gene and a region downstream of the endogenous target gene, which is accomplished by incorporating 500-1000 base pairs of the target sequence and 500-1000 base pairs of the sequences located 3′ to the target gene itself into the vector construct flanking a positive selectable marker or a positive selectable marker genetically linked to a negative selectable marked. In particular embodiments 500-1000 base pairs of sequence located upstream of the target gene is cloned in between the selectable marker and the down stream region. In particular embodiments, the target gene is replaced by one or more of the selectable markers as described in the present invention. For example, a vector for a targeted gene deletion would include a pyrF, tdk, hpt or cat gene sequence, or any combination of linked marker gene sequences, flanked by sequence that is homologous to the target gene. Such a vector, when transformed into a thermophilic strain, will recombine with the target gene and result in the replacement of the target gene with the selectable marker(s) and the duplicated upstream region. A variation of this can be achieved in which a portion of the target gene is omitted and either the upstream or downstream region is duplicated on the plasmids.

The targeted gene deletion and plasmid loss can be selected for using the counterselectable marker located outside the engineered upstream and downstream flanks. This selection creates an allelic replacement of a portion of the target gene with the upstream region and a gene containing a positive and negative selection as in pyrF or a positive selectable marker linked to a negative selectable marker, as in cat linked to hpt/tdk. Recombination between the duplicated upstream regions results in a clean deletion and is selected for using the negative selection against the marker(s) that replaced the portion of the target gene.

Alternatively, a vector can be constructed such that the target gene is replaced by a negative or dual (has both positive and negative selection) selectable marker gene or cassette, such as pyrF, tdk, or hpt alone or linked to a positive selectable marker such as an antibiotic resistance gene, such as cat, or an additional dual selectable marker, such as pyrF, tdk, or hpt. Such a vector would comprise, for example, a cat and pyrF gene flanked by sequence that is homologous to the target gene. A selectable marker such as pyrF, tdk or hpt can be included on such a vector located outside of the flanking sequence. In this way, once the cat-pyrF cassette has been integrated into the genome replacing the target gene, the loss of the plasmid can be selected by the use of positive and/or negative selection, depending on which selectable marker is located outside of the flanking region. Selection for the loss of a plasmid once a targeted gene deletion has been made results in the creation of a marked strain that has the gene of interest replaced by a cassette or individual gene with negative selection. A second vector can be made that has the same flanking DNA that was used in the example above and a selectable marker such as pyrF, tdk or hpt can be included on such a vector located outside of the flanking sequence for plasmid loss. In this way, once the plasmid recombines with the chromosome, the cassette with negative selection, such as the cat-pyrF cassette described above, that has been integrated into the genome replacing the target gene, can be selected against. Upon loss of the plasmid using the negative marker located outside of the flanking region, a plasmid-free strain with a clean deletion of the target gene can be generated.

In additional embodiments, a vector can be constructed such that the endogenous target gene is replaced by a nonfunctional version of the target gene, a mutated version of the target gene, or a non-selectable sequence. Such a vector can also include a counterselectable marker located outside the engineered upstream and downstream flanks. After plasmid loss selection, as described above, an unmarked strain harboring a disruption or mutation of the target gene can be generated in an unmarked strain.

A marked or unmarked strain of the invention that harbors a targeted gene deletion can be further modified to include a second targeted gene deletion by virtue of the ability to transform this unmarked strain with a vector having any selectable marker of the present invention. In this way, selectable markers can be “recycled” or reused to further engineer a thermophilic organism.

Targeted gene deletions of thermophilic hosts can be made to generate a strain capable of increased ethanol production, increased lactate production or increased acrylate production, for example. Target genes include, but are not limited to, lactate dehydrogenase (ldh), hydrogenase, phosphotransaceytlase (pta), acetate kinase (ack), nitrogenase, pyruvate formate lyase (pfl), methylglyoxal synthase, and Spo0A, as well as other genes involved in central metabolism, stress response, and carbohydrate utilization.

In one embodiment, the invention provides for the creation of a thermophilic strain containing a deletion of the pyrF gene. Demonstration of the use of pyrF as a positive and negative selectable marker is conducted by reintroduction of the pyrF gene in such a strain, as discussed further below.

The invention also provides for the creation of a thermophilic strain expressing the thymidine kinase (tdk) gene. In additional embodiments, the introduction of the tdk gene and demonstration of its use as negative selectable marker is conducted, as discussed further below.

The invention additionally provides for the creation of a thermophilic strain containing a deletion of the hypoxanthine phsophribosyltransferase (hpt) gene. In additional embodiments, the reintroduction of the hpt gene and demonstration of its use as a positive and negative selectable marker is conducted, as discussed further below. In further aspects of the invention, a tdk, hpt and/or additional selectable markers, including an antibiotic resistance gene can be further introduced into an hpt-deleted strain. Thus, the invention provides for the incorporation of hpt, tdk and/or an antibiotic resistance gene into a single tool (or vector) for making markerless clean deletions in thermophiles.

The invention also provides for the creation of strains containing a combination of the selectable markers described above.

### Selectable Marker Genes

### PyrF

The pyrF gene encodes the pyrimidine biosynthetic enzyme orotidine-5′-monophosphate (OMP) decarboxylase. Its homology to the Saccharomyces cerevisiae URA3 gene allows for adaptation of the URA3 selection system, allowing both positive and negative selection of the marker. URA3 encodes an enzyme, orotidine-5′-phosphate decarboxylase (ODCase), that can catalyze the conversion of 5-fluoroorotic acid (5-FOA) into a highly toxic compound. Thus, counterselection works on the basis that the presence of URA3 confers sensitivity to 5-FOA, while URA3-negative cells are 5-FOA resistant. Alternatively, URA3 as a positive selection marker works based on the ability of an exogenous or plasmid-borne URA3 gene complementing uracil auxotrophy of a URA3-negative strain.

The present invention provides for a thermophilic bacterial system that utilizes the selection capabilities of the pyrF gene. One aspect of the invention provides for the creation of thermophilic strain in which the pyrF gene has been deleted (ΔpyrF). Deletion of pyrF results in uracil auxotrophy, and thus growth of such a strain requires uracil to be supplemented in the growth medium, or alternatively, expression of an exogenous or plasmid-borne pyrF gene. In such a system, positive selection for maintenance of a plasmid containing pyrF can be achieved by subjecting the ΔpyrF thermophilic strain, e.g., ΔpyrF C. theremocellum, to media lacking uracil. Negative selection can be achieved by subjecting the ΔpyrF thermophilic strain containing a plasmid borne copy of pyrF to media containing uracil and the antimetabolie 5-fluoro-orotic acid (5-FOA).

As discussed above, a ΔpyrF thermophilic strain is a uracil auxotroph. While such a strain has the advantage of being utilized for both positive and negative selection, due to its auxotrophy, such a strain can sometimes have diminished growth which may complicate strain development. This can be particularly relevant when a targeted gene deletion also causes diminished growth. A strain that is being engineered to increase ethanol production, for example, by deletion of the genes for ldh or pta, can have diminished growth. Thus, the use of a pyrF selection system can further affect the growth capability of such a strain. Furthermore, supplementation of such a strain with uracil can complicate directed evolution and bioprocess studies. Additionally, it is possible that uracil deficiency interferes with pyrimidine synthesis such that stably maintaining large plasmids is difficult.

The use of at least a single additional or alternative negative and/or positive selectable marker would be advantageous in achieving an unmarked gene deletion, or having the capability of recycling genetic markers. The present invention provides for such additional markers, such as tdk and hpt, as discussed further below.

### Tdk

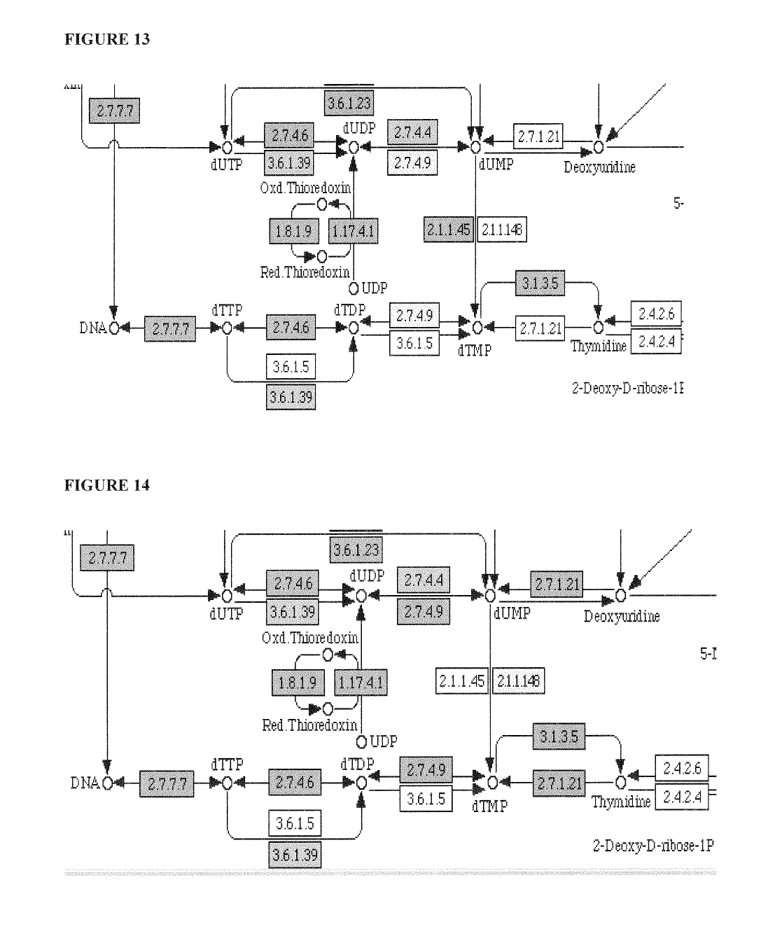

It is known that the thermophilic organism C. thermocellum lacks the tdk gene. In FIG. 13, the enzyme activity encoded by the tdk gene is indicted by EC#2.7.1.21. The white boxes indicate enzyme activities for which bioinformatic studies did not yield a corresponding encoded open reading frame (orf) in the genome. The activity for tdk (2.7.1.21) is such an example. It is further known that the thermophilic organisms T. saccharolyticum contains a tdk gene. In FIG. 14, the enzyme activity encoded by the tdk gene is indicted by EC#2.7.1.21. Green boxes indicate enzyme activities for which bioinformatic studies yield a corresponding encoded open reading frame (orf) in the genome. The activity for tdk (2.7.1.21) is such an example.

The advantages of using a tdk gene as a negative selection marker are numerous. First of all, the tdk gene is small. The T. saccharolyticum tdk gene, for example, is 579 base pairs in length, making it less burdensome to incorporate into cloning strategies. Secondly, as noted above, the tdk is not a native gene to C. thermocellum. Thus, unlike pyrF, an accompanying mutant devoid of the chromosomal copy of the gene does not need to be generated. Finally, unlike pyrF, the selection does not require an auxotrophy so there is not impairment of growth.

The tdk gene can also be used as a positive selectable marker in thermophilles. This selection uses inhibitors of dihydrofolate reductase such as aminopterin or trimethoprim, together with hypoxanthine and/or thymidine, which are intermediates in DNA synthesis. The inhibition of dihydrofolate reductase by aminopterin or trimethoprim blocks de novo DNA synthesis, which is required for growth of the cells. Thymidine and hypoxanthine are intermediates that allow cells to use the pyrimidine and purine salvage pathways, respectively. C. thermocellum does not have a tdk gene and thus lacks a true pyrimidine salvage pathway so trimethoprim is lethal to the cell. When transformed into C. thermocellum, the tdk gene product can thus be positively selected for in the presence of trimethoprim and thymidine.

The tdk gene to be introduced into C. thermocellum can be derived from any organism expressing a native tdk. In particular, the tdk can be derived from another thermophile, such as T. saccharolyticum, which can make it more suitable for use in C. thermocellum as compared to a tdk gene from a mesophile, such as E. coli or S. gordonii.

An exemplary gene encoding a Tdk enzyme for use in the invention is shown below. The DNA sequence of this 570 bp orf is as follows:

(SEQ ID NO: 1)
ATGTATGGGCCTAAAGACCACGGGTACATAGAAGTTGTAACTGGTCCCATGT
TCAGTGGAAAAAGTGAGGAACTTATAAGAAGGATAAAGAGAGCTAAGATTGCCAGG
CAAAAAGTTCAGGTTTTCAAACCGGCTATAGACGATAGGTATTCCATAGACAAAGTC
GTATCTCATAACGGCGACAACATGCACGCCATTGCCATAGTAAAGGCTTCTGACATA
TTGGCTTATGCTGAAGAAGATACGGATGTATTTGCTATAGATGAAGTTCAATTTTTTG
ATTCTGAAATAGTCGACATCGTAAAAGAGATTGCCGATAGCGGGAAAAGAGTCATAT
GTGCAGGGCTTGACATGGACTTTAGAGGTGAACCATTTGGTCCGACTCCAGAATTGA
TGGCCATAGCTGAATTTGTCGATAAGCTTACTGCTATATGCATGAAGTGTGGCAATCC
TGCTACTCGCACGCAAAGGCTTATAAATGGGAAGCCTGCCAATTACGACGATCCCAT
TATAATGGTTGGAGCAAAGGAGTCTTATGAAGCAAGGTGTAGAAAGTGCCATGAAGT
CCCGCGGACTTAA

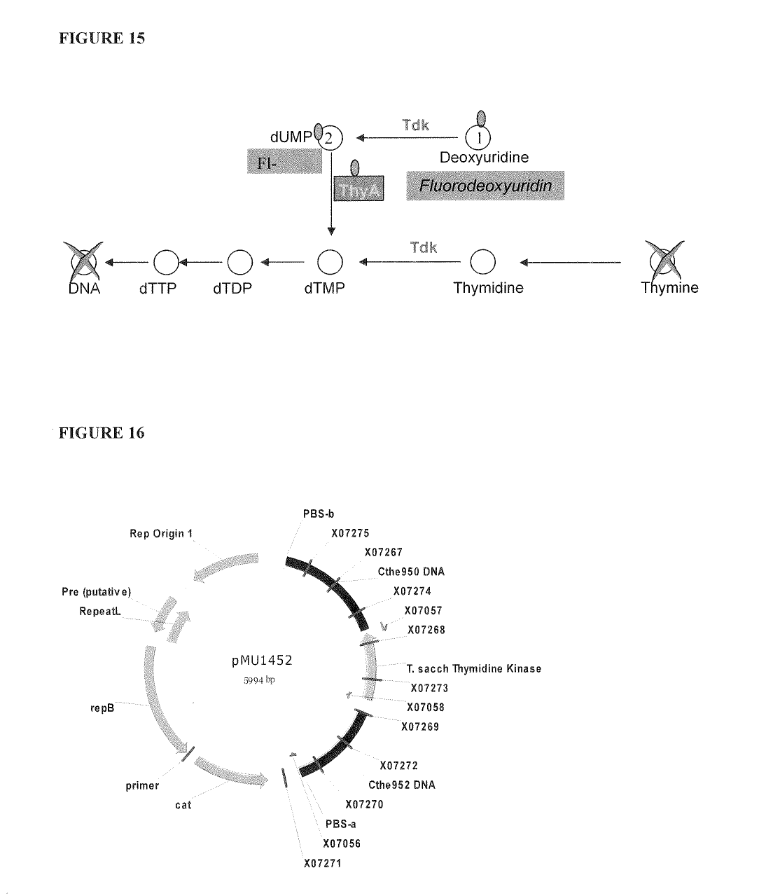

The presence of the tdk gene can be detected by negative selection, and in particular, by the addition of fluorodeoxyuridine (FUDR). The mechanism by which FUDR is a toxic antimetabolite in the presence of the enzyme activity encoded by the tdk gene is depicted in FIG. 15. Fluorodeoxyuridine is a toxic analog of deoxyuridine. As shown in FIG. 15, fluorodeoxyuridine is converted to Fluoro-BUMP by Tdk. Fluoro-dUMP is a covalent inhibitor of thymidylate synthestase (ThyA) which is required for DNA synthesis. Therefore, in a cell expressing Tdk, the addition of FUDR will result in an inhibition of DNA synthesis, and thus cell death. Thus, the presence of tdk is detectable upon addition of FUDR as represented by a lack of colony formation.

Thus, the advantage of using tdk as a negative selection marker in C. thermocellum is that higher order, targeted gene deletions can be achieved, one goal being a high yielding ethanologen.

### Hpt

Many bacterial cells contain two distinct pathways for creating the purine intermediates necessary for DNA synthesis. The de novo purine synthesis pathway is typically responsible for a majority of this production. This pathway is a long, energy intensive, and makes purines “expensive” for the cell to manufacture. Under conditions where a culture is dividing rapidly or nutrients are limiting, cells conserve energy by replenishing the purine pool with the salvage pathway. In laboratory conditions, this pathway is rarely necessary. The hyoxanthine phosphoribosyltransferase gene encodes a protein that operates within the salvage pathway, and thus is a good candidate for use as a genetic tool.

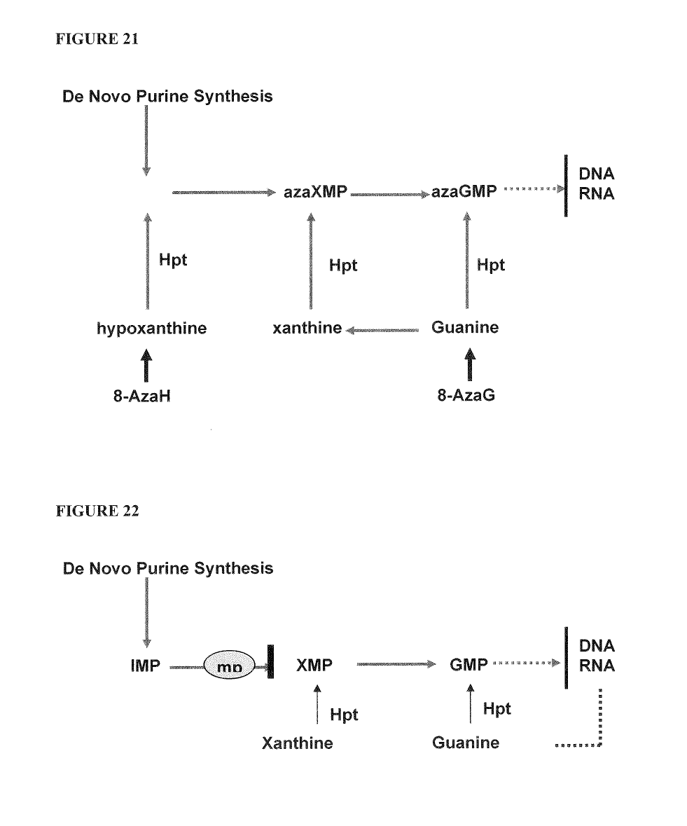

The hpt gene can be used as both a positive and negative selectable marker. The hpt gene can be used as a negative selectable marker by utilizing anti-metabolites such as 8-azahypoxanthine (8-azaH). When the hpt gene is present and the gene product expressed, the anti-metabolite is incorporated into toxic purine intermediates and the cells die (FIG. 21). When the hpt gene is absent, the gene product is not expressed and the drug has no effect because the anti-metabolite itself is not toxic without the hpt gene product.

The hpt gene can be used as a positive selectable marker which is relatively rare in the absence of an auxotrophy. In a Δhpt strain the cells make purines through the de novo biosynthesis pathway. The de novo pathway itself can be inhibited by compounds such as mycophenolic acid (MPA) which inhibit the enzyme inosine 5′-monophosphate dehydrogenase (EC 1.1.1.205). In a wild type strain, MPA has little or no effect because the cell can still utilize the salvage pathway. Since a Δhpt strain is lacking the salvage pathway, mycophenolic acid becomes lethal to the cell since it can no longer synthesize purines. In a Δhpt strain, plasmids expressing a functional Hpt can be positively selected for on MPA. (FIG. 22.)

Deletion of the hpt gene affects only the purine salvage pathway and a growth defect is not expected. The present invention provides for the use of hpt alone or together with tdk and/or an antibiotic resistance gene to develop a thermophillic genetic tool applicable in a thermophilic organism, such as T. saccharolyticum or C. thermocellum. For example, because the hpt gene can be used as both a positive and negative selectable marker, this can potentially eliminate the need to link it to an antibiotic resistance gene.

The molecular vectors comprising the selectable markers described above can be tailored to meet the distinct requirements of the thermophilic organism to be engineered, but will be very similar in design, function, and technical application.

The gene encoding the T. saccharolyticum Hpt enzyme is designated by oak ridge national labs as or 0940 and is located on Contig7.

Or0940:
(SEQ ID NO: 2)
atggaaaatttatcaaaagacatcgatgaaattttgatcacagaagaagaacttaaggaaaagataaaagagcttggg
aggcaaatcacaaaagactacaaagggaaaaatttgatgttggtaggagttttaaaaggtgctttaatgtttatggctgatttgtcaa
gacacatagatttgcctttatcacttgattttatggctgtttccagctatggaagctcaactcattcatcaggaatagtaaagataatca
aagatcttgatataagcatagaaggcaaagatgttctgattgtggaagacataattgacagcggtttgactttgtatacttaaggga
aactttacttggaaggaagccaaaaagcctgaaaatatgcacaatattagacaaaccggagagaagagaagcatctgtaaaagt
cgattatgtaggatttaagatacctgataagtttgtcgtgggttatggattggactttgatgaaaagtacaggaaccttccttttatagg
cgttttgaaacctgaaatgtacagctaa

The genome sequence of C. thermocellum strain 1313 is not yet available so there is not a gene identifier. The Hpt enzyme in C. thermocellum strain 27405 is designated by oak ridge national labs as Cthe_{—}2254. Manual sequence verification showed 100% homology between the two genes.

Cthe_2254:
(SEQ ID NO: 3)
atgataaatcaaattaaagaaattttggttaccagagaggaacttaaaaacaacgctaaagagttgggaaagaggattt
ccagtgactatgaaggaaaagagcttgtcctgataggggtgttaaaaggaggagtggtattttttgccgacttaataagggaaata
accatacccattgatgtggatttcatatcggtgtcaagttacggcaattccaccaaatcatcgggggttgtgcgtataataaaagac
atcgatatagatataaccaacaagcatgtccttatcgttgaagacttggtggatacaggtcttacgctgcattatctgaaaagcatgtt
tgaagccagaggacccaaagatgtaaaaatatgcaccgcccttgacaaaccgtcaaggagaaaggttgatttggaaatagattat
aaaggtatcacaataccggataagtttgtggtgggctatggattggattatgcggaaaaatacagaaatctcccggatgtgtgcgt
gctggattcgtctgtttatacggacaaagaagatatggactaa

### Vectors and Host Cells

The present invention also relates to vectors which include selectable markers of the present invention, host cells which are genetically engineered with vectors of the invention and the production of polypeptides of the invention by recombinant techniques.

Host cells are genetically engineered (transduced or transformed or transfected) with the vectors of this invention which can be, for example, a cloning vector or an expression vector. The vector can be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

The appropriate selectable marker sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively associated with an appropriate expression control sequence(s) (promoter) to direct mRNA synthesis. Any suitable promoter to drive gene expression in the host cells of the invention can be used, including the cbp, gapDH, pyrF, promoter from C. thermocellum. Additionally, the E. coli, lac or trp, and other promoters known to control expression of genes in prokaryotic or lower eukaryotic cells can be used. The expression vector also contains a ribosome binding site for translation initiation and a transcription terminator. The vector can also include appropriate sequences for amplifying expression, or can include additional regulatory regions.

In addition, the expression vectors may contain one or more selectable marker genes to provide a phenotypic trait for selection of transformed host cells such as pyrF, tdk and/or hpt.

The vector containing the appropriate selectable marker sequence as used herein, as well as an appropriate promoter or control sequence, can be employed to transform an appropriate thermophilic host to permit the host to express the protein.

Thus, in certain aspects, the present invention relates to host cells containing the above-described constructs. The host cell can be an anaerobic thermophilic bacterial cell, including an anaerobic xylanolytic and/or cellulolytic host cell. The selection of an appropriate host is deemed to be within the scope of those skilled in the art from the teachings herein.

Major groups of thermophilic bacteria include eubacteria and archaebacteria. Thermophilic eubacteria include: phototropic bacteria, such as cyanobacteria, purple bacteria, and green bacteria; Gram-positive bacteria, such as Bacillus, Clostridium, Lactic acid bacteria, and Actinomyces; and other eubacteria, such as Thiobacillus, Spirochete, Desulfotomaculum, Gram-negative aerobes, Gram-negative anaerobes, and Thermotoga. Within archaebacteria are considered Methanogens, extreme thermophiles (an art-recognized term), and Thermoplasma. In certain embodiments, the present invention relates to Gram-negative organotrophic thermophiles of the genera Thermus, Gram-positive eubacteria, such as genera Clostridium, and also which comprise both rods and cocci, genera in group of eubacteria, such as Thermosipho and Thermotoga, genera of Archaebacteria, such as Thermococcus, Thermoproteus (rod-shaped), Thermofilum (rod-shaped), Pyrodictium, Acidianus, Sulfolobus, Pyrobaculum, Pyrococcus, Thermodiscus, Staphylothermus, Desulfurococcus, Archaeoglobus, and Methanopyrus.

Some examples of thermophilic microorganisms (including bacteria, prokaryotic microorganisms such as fungi), which may be suitable for the present invention include, but are not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Thermoanaerobacterium thermosaccarolyticum, Thermoanaerobacterium saccharolyticum, Thermobacteroides acetoethylicus, Thermoanaerobium brockii, Methanobacterium thermoautotrophicum, Pyrodictium occultum, Thermoproteus neutrophiles, Thermofilum librum, Thermothrix thioparus, Desulfovibrio thermophilus, Thermoplasma acidophilum, Hydrogenomonas thermophilus, Thermomicrobium roseum, Thermus flavas, Thermus ruber, Pyrococcus furiosus, Thermus aquaticus, Thermus thermophilus, Chloroflexus aurantiacus, Thermococcus litoralis, Pyrodictium abyssi, Bacillus stearothermophilus, Cyanidium caldarium, Mastigocladus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarius, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinata, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulars, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrificans, Streptomyces thermovulgaris, Pseudonocardia thermophila, Thermoactinomyces vulgaris, Thermoactinomyces sacchari, Thermoactinomyces candidas, Thermomonospora curvata, Thermomonospora viridis, Thermomonospora citrina, Microbispora thermodiastatica, Microbispora aerata, Microbispora bispora, Actinobifida dichotomica, Actinobifida chromogena, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, variants thereof, and/or progeny thereof.

In certain embodiments, the present invention relates to thermophilic bacteria of the genera Thermoanaerobacterium or Thermoanaerobacter, including, but not limited to, species selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brockii, variants thereof, and progeny thereof.

In particular embodiments, the host cell is Clostridium thermocellum or Thermoanaerobacterium saccharolyticum. In additional embodiments, the host cell is a xylanolytic host of the genus Anaerocellum, Caldicellulosiruptor or Moorella.

The present invention also includes recombinant constructs comprising one or more of the selectable marker sequences as broadly described above. The constructs comprise a vector, such as a plasmid or viral vector, into which a sequence of the invention has been inserted, in a forward or reverse orientation. In one aspect of this embodiment, the construct further comprises regulatory sequences, including, for example, a promoter, operably associated to the sequence. Large numbers of suitable vectors and promoters are known to those of skill in the art, and are commercially available. The following vectors are provided by way of example only.

Introduction of the construct in host cells can be done using methods known in the art. Introduction can also be effected by electroporation methods as described in U.S. Prov. Appl. No. 61/109,642, filed Oct. 30, 2008, the contents of which are herein incorporated by reference.

### EXAMPLES

### Example 1

### Positive and Negative Selection Using a C. thermocellum pyrF Knockout

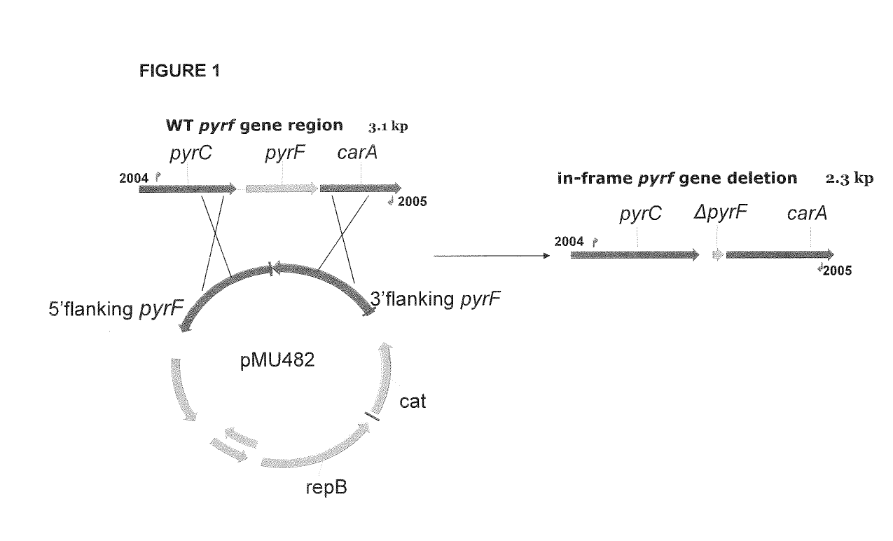

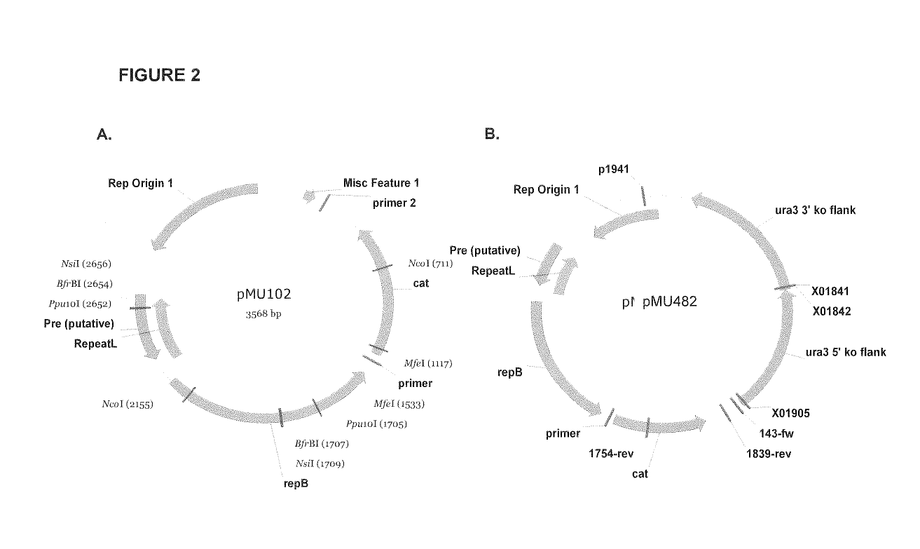

Homologous recombination was utilized to make an in-frame deletion of the pyrF gene. A depiction of the expected recombination events and the resulting pyrF deletion is shown in FIG. 1. To knockout the pyrF gene, a replicative knockout vector was constructed which contained sequences homologous to upstream and downstream sequences of the pyrF gene. The created plasmid is referred to as pMU482. The control plasmid, pMU102, does not contain sequences homologous to upstream or downstream sequences of the pyrF gene. The pMU482 and pMU102 plasmids are depicted in FIG. 2.

Creation of C. thermocellum pyrF Deletion

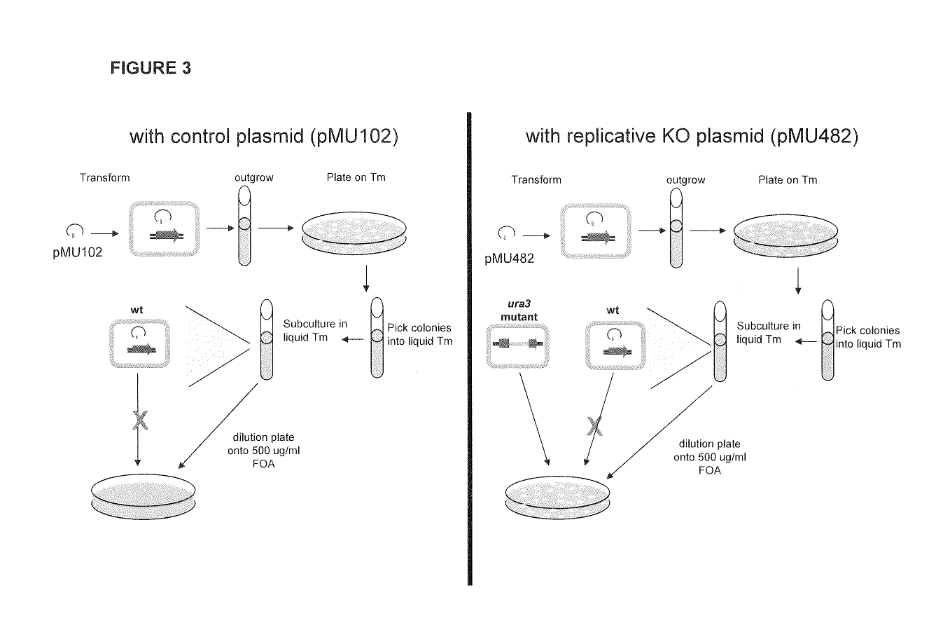

Plasmid pMU482 was transformed into the wild type C. thermocellum strain 1313 and selected on a rich media containing thyamphenicol (at the concentration of 6 μg/ml) to select for cells containing the cat marker encoded on the plasmid. A schematic displaying the creation of the knockout vector is depicted in FIG. 2. Several colonies that resulted from the selection scheme were selected for PCR analysis to confirm that the plasmid had been transformed into the cells. A transformed colony was diluted into liquid, rich medium containing thyamphenicol at the concentration of 6 μg/ml and grown overnight. A culture containing pMU102 (control plasmid) was also grown similarly. The next day, each culture was serially diluted by ten fold (up to 10⁻³) using fresh, rich media and 100 μL of the undiluted culture. Each of the dilutions were plated on rich media containing 500 μg of 5-FOA. A schematic of this plating strategy is depicted in FIG. 3. Colonies that contained the pyrF disruption were visible after plating on to 5-FOA, whereas colonies containing wild-type pyrF did not survive.

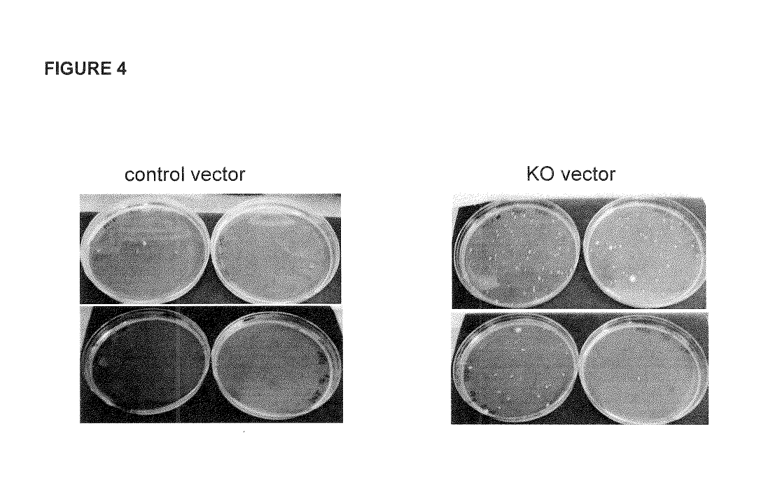

When the control pMU102 plasmid was transformed into C. thermocellum, only a few colonies appeared, most likely representing cells that gained spontaneous resistance to 5-FOA. When the pMU482 knockout vector was used to transform C. thermocellum, a much higher number of colonies appeared compared to the control. Results of the experiment are shown in FIG. 4.

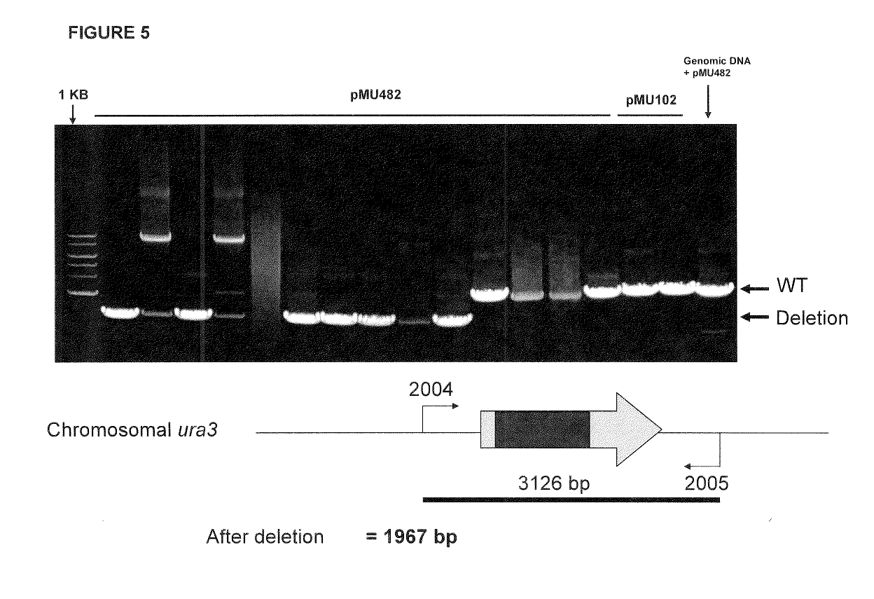

PCR screens were performed to verify that the pyrF gene had been deleted from the chromosome. The recombination events allowing the pyrF gene deletion are depicted in FIG. 1. The expected bands of the deleted or wild-type gene are indicated in FIG. 5. PCR analysis confirmed that six of the selected colonies contained the pyrF deletion.

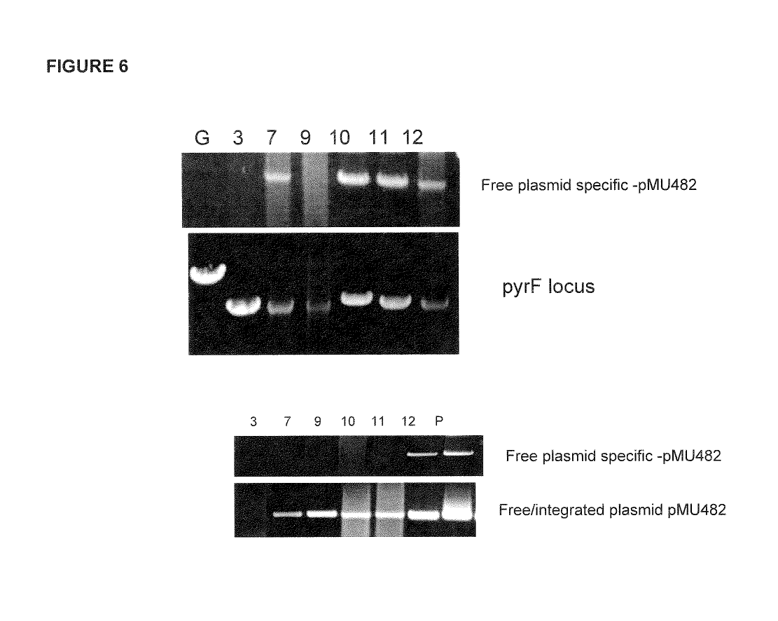

Colonies were also tested by PCR amplification to confirm that the ΔpyrF strain was plasmid free. Results of the PCR screen depicted in FIG. 6 indicate that colony #3 showed a loss of the knockout plasmid.

Positive Selection Using a C. thermocellum pyrF Knockout

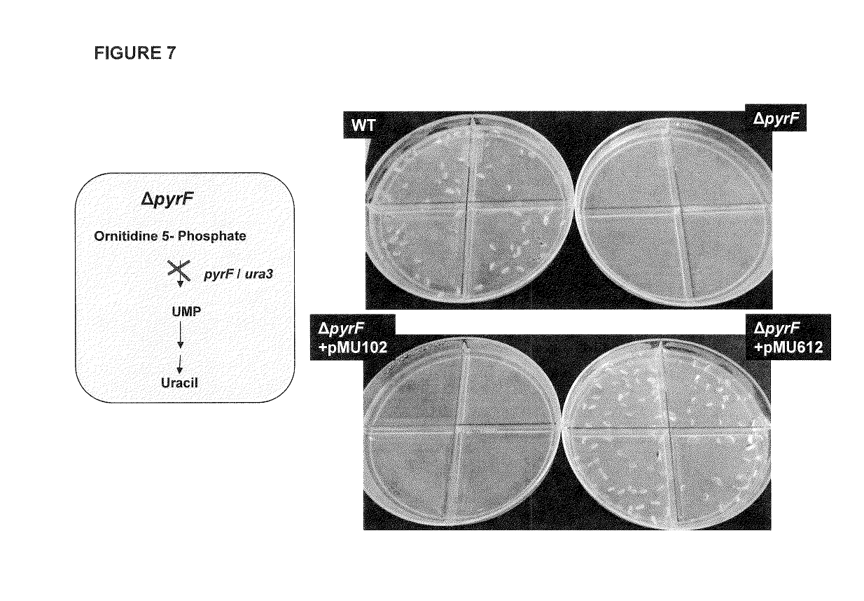

The plasmid-free C. thermocellum ΔpyrF strain created above was further tested to confirm that positive selection could be appropriately applied. Cells were grown and plated on uracil minimal media. A C. thermocellum ΔpyrF strain is unable to grow on minimal media (lacking uracil), whereas a wild-type strain can. As shown in FIG. 7, the wild-type strain plated on minimal media formed colonies, whereas the ΔpyrF strain did not. When the ΔpyrF strain was transformed with the pMU102 control plasmid (lacking sequence encoding for pyrF), as expected the pyrF deficiency was not complemented. However, when the ΔpyrF strain was transformed with pMU612, a complementing plasmid containing a pyrF gene under control of the cbp promoter, colonies were formed on medium lacking uracil. This data demonstrates that pyrF can be successfully utilized as a positive selection marker in C. thermocellum using uracil minimal media.

Negative Selection Using a C. thermocellum pyrF Knockout

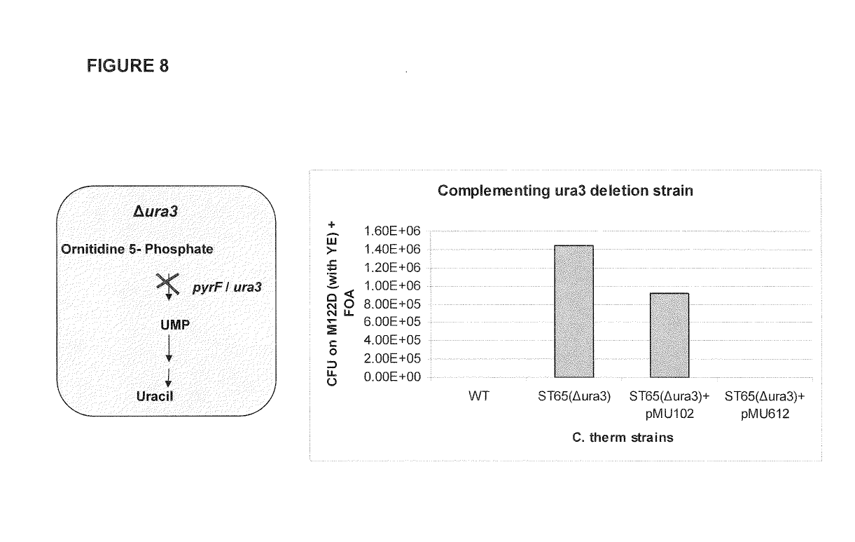

The plasmid-free C. thermocellum ΔpyrF strain created above was tested to confirm that negative selection could be appropriately applied. Cells were grown and plated using media containing the toxic analog 5-fluoroorotic acid (5-FOA). The protein product of the pyrF gene converts 5-FOA into a toxic compound. Thus, the C. thermocellum ΔpyrF strain should be resistant to 5-FOA, but the wild type or a complemented strain should be susceptible to 5-FOA and unable to grow. As shown in FIG. 8, the ΔpyrF strain was able to grow as expected, as well as the same strain transformed with control plasmid pMU102. In contrast, the ΔpyrF strain transformed with the complementing plasmid pMU612 expressing pyrF showed no growth. This data demonstrates that pyrF can be successfully utilized as a negative selection marker in C. thermocellum.

### Example 2

### Positive and Negative Selection in Targeted Gene Deletions of C. thermocellum Using pyrF

The ability of pyrF to be utilized as a positive and negative selection marker in C. thermocellum led to the utilization of this marker for the creation of strains with a targeted gene deletion. One such target gene phosphotransacetylase (pta) is involved in the conversion of acetyl-CoA to acetate. The production of a modified organism harboring a pta deletion would be advantageous since it would prevent acetate production, thereby channeling the carbon flux towards increased ethanol production. Deletion of pta would facilitate the ultimate goal of making a homoethanologen strain (in conjunction with the deletion of other byproduct pathway genes such as ldh).

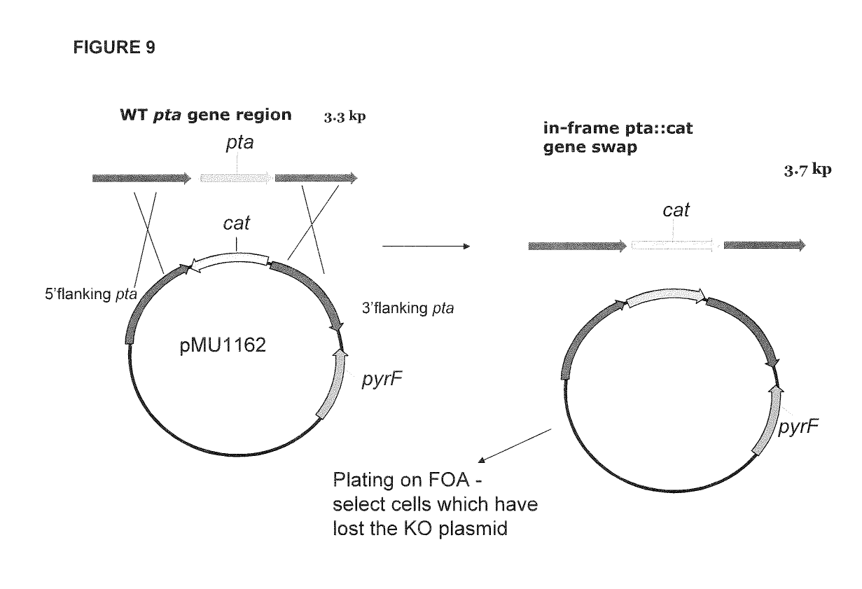

To knockout the pta gene, a knockout vector, pMU1162, was constructed which contained sequences homologous to upstream and downstream sequences of the pta gene with a chloramphenicol acetyltrasnferase (cat) gene located between the two flanking sequences. The knockout vector also contained a pyrF gene located outside of the flanking region sequence. A C. thermocellum strain was transformed with this plasmid. The transformation positive colonies were grown in rich media containing thyamphenicol (Tm6 mg/ml). Next day the cultures were plated on media containing thyamphenicol and 5-FOA. By homologous recombination, the endogenous pta was replaced with cat. A diagram depicting the vectors and the recombination events is shown in FIG. 9. The plating on 5-FOA of the pta knockout strain resulted in the selection of cells that had lost the plasmid due to the presence of the pyrF gene. As described above, the presence of the pyrF gene product results in the incorporation of the toxic analog during uracil synthesis resulting in cell death.

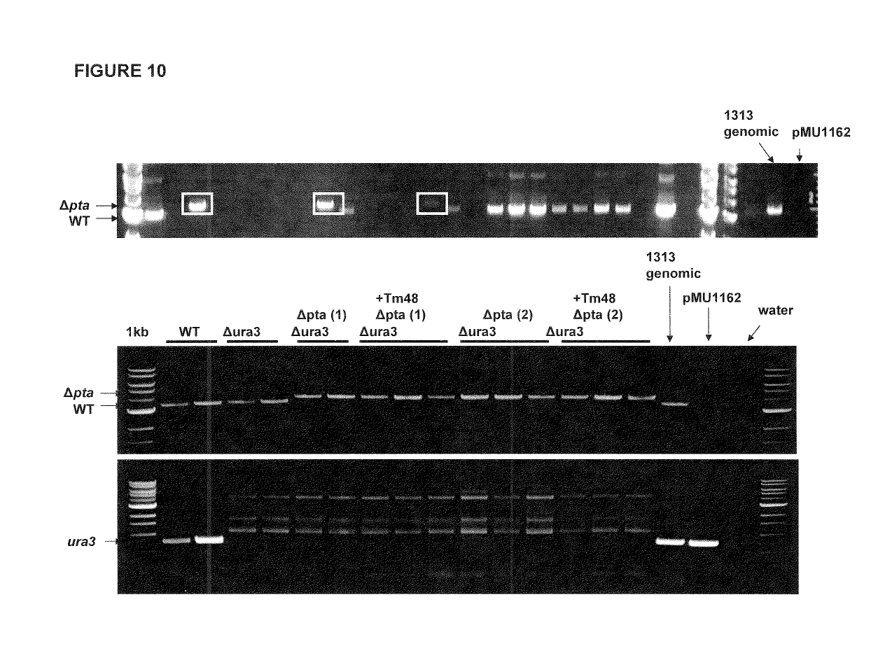

PCR analysis was performed to confirm that deletion of the pta gene had been successfully achieved. As shown in FIG. 10, strains in which pta was deleted and replaced with the cat gene were created. Negative selection utilizing 5-FOA resulted in strains cured of the plasmid.

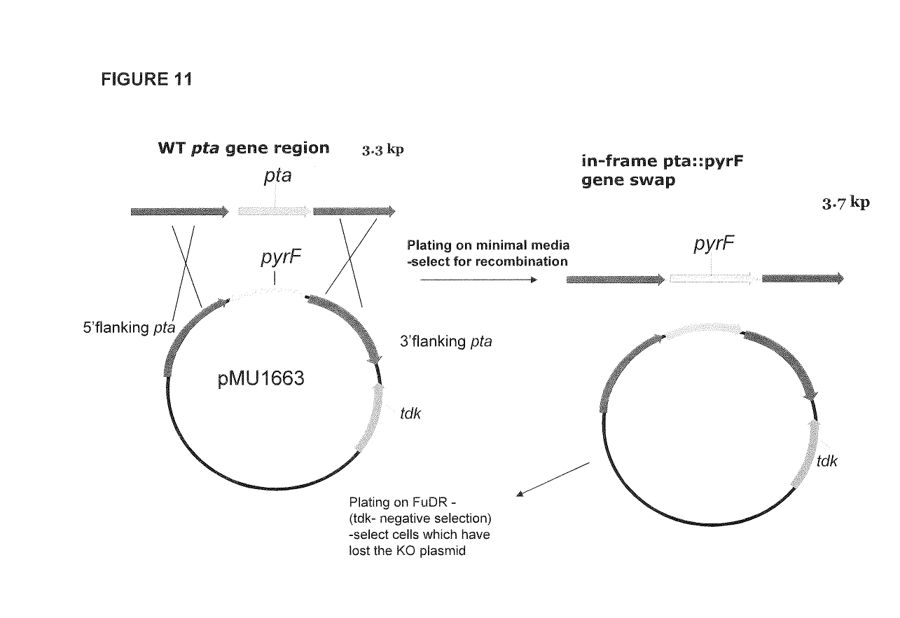

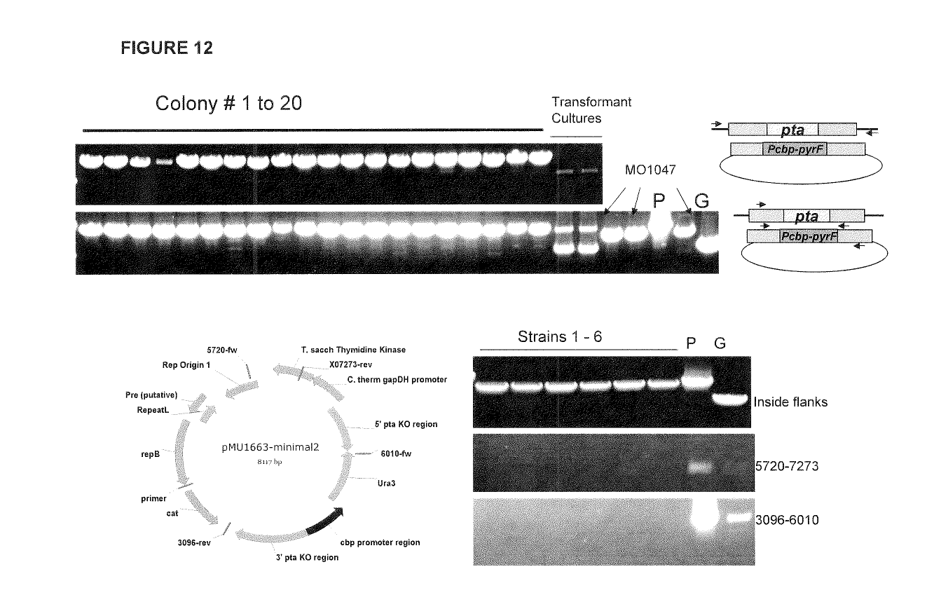

The pta gene was also knocked out utilizing a vector that contained pyrF gene sequences located between the two flanking sequences, rather than the chloramphenicol acetyltransferase (cat) gene sequence described above. This vector, referred to as pMU1663, is depicted in FIG. 11. The pMU1663 vector also contained a tdk gene located outside of the flanking region sequence to be used as an additional selectable marker. A C. thermocellum strain was transformed with this plasmid. By homologous recombination, the endogenous pta was replaced with pyrF. Plating on uracil minimal media of the pta knockout strain resulted in the selection of cells that had successfully integrated pyrF at the pta locus. As shown in FIG. 12, transformants harboring the integrated pyrF were identified by PCR analysis. The tdk gene was used as a negative selectable marker to select for cells that have lost the knockout plasmid.

### Example 3

### Negative Selection Utilizing tdk Creation of a C. thermocellum Strain Containing T. saccharolyticum tdk

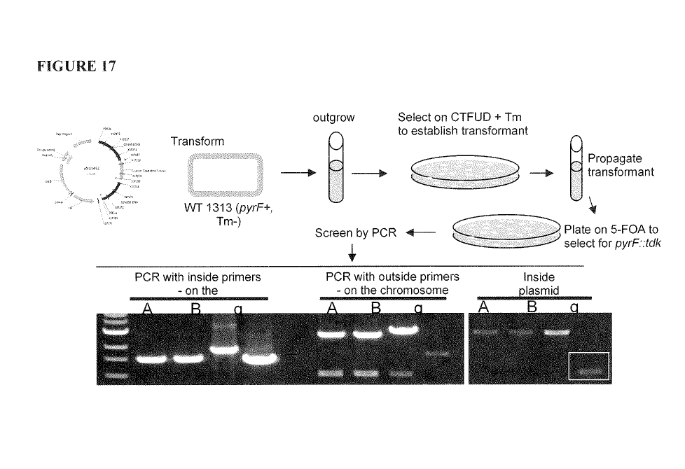

To stably express the T. saccharolyticum tdk gene in C. thermocellum, allelic replacement using pyrF was performed. Plasmid pMU1452, as depicted in FIG. 16, was designed to replace the native C. thermocellum pyrF gene with the T. saccharolyticum tdk gene. The resulting strain is designated pyrF::tdk. The pyrF locus was chosen as the site for allelic replacement because the antimetabolite 5-FOA can be used to select for loss of the pyrF gene, as discussed above. The sequence of pMU1452 corresponds to SEQ ID NO: 4. The strategy used to generate the tdk strain and the results of PCR screening are shown in FIG. 17. PCR screening of colonies A and B demonstrates that the pyrF gene was successfully replaced with tdk.

### Negative Selection Using tdk

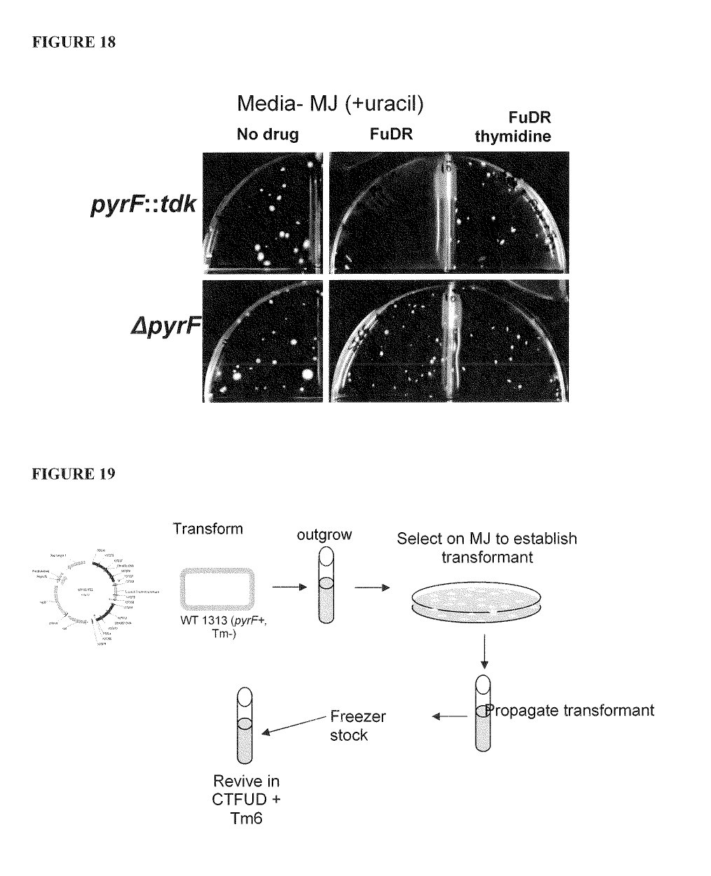

C. thermocellum strains carrying the T. saccharolyticum tdk gene should be sensitive to fluorodeoxyuridine (FUDR) whereas those that do not have the tdk gene should be resistant to FUDR. To this end, the C. thermocellum pyrF::tdk strain and the control C. thermocellum ΔpyrF strain were plated in the presence of 10 μg/ml FUDR. For each culture, a 10 dilution was used for the plating. Results of the plating are shown in FIG. 18. The pyrF::tdk strain did not grow in the presence of FUDR, as expected, but did grow with the addition of thymidine, included to counter the effects of FUDR. In contrast, the ΔpyrF strain that did not contain an endogenous integration of tdk survived in the presence of FUDR. These results demonstrate that the T. saccharolyticum tdk gene can be used successfully as a negative selectable marker in C. thermocellum.

Curing of the C. thermocellum pyrF::tdk Strain to Remove the Plasmid

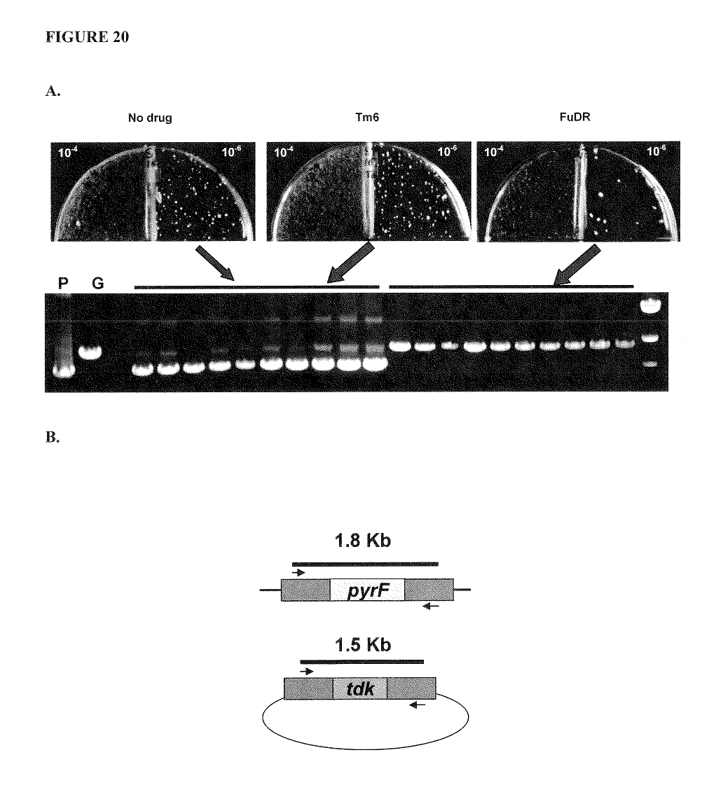

Wild-type C. thermocellum harboring the pMU1452 vector was assayed for plasmid curing in the presence of FUDR. A cartoon schematic showing the process is shown in FIG. 19. The revived culture was plated and screened for the presence of the plasmid by PCR. Results of the PCR screen are depicted in FIG. 20A.

A single set of primers was used that bind to both the plasmid and the chromosome. The diagram in FIG. 20B depicts the annealing of each primer on both chromosome and the plasmid. The region between the primer binding sites on the chromosome is bigger than that on the plasmid. Therefore, PCR amplification using this primer set results in two different sized bands depending on whether genomic DNA is used as a template (giving band size of 1.8 Kb) or plasmid DNA is used as template (giving the band size of 1.5 Kb). Control PCR reactions on plasmid DNA (lane labeled “P”) and genomic DNA (lane labeled “G”) were performed. PCR performed on colonies harboring the plasmids will amplify two bands (one amplifying the chromosomal region −1.8 Kb and the other amplifying plasmid region −1.5 Kb) with preferential amplification of the smaller plasmid target. PCR performed on colonies that have lost the plasmid will amplify a single band corresponding to the larger chromosomal target. The results above show that FUDR resistant colonies have lost the plasmid, whereas colonies growing in the presence of Tm6 and no FUDR still contain plasmid.

### Example 4

### Creation of a C. thermocellum hpt Knockout

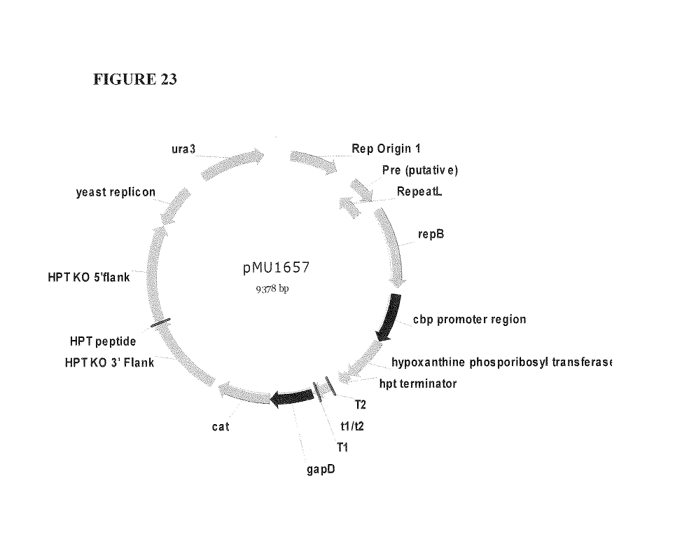

To knockout the hpt gene a vector was constructed which contained a region homologous to ˜1 kb upstream and downstream of the hpt gene. Additionally, a copy of the hpt gene expressed from the cellobiose phosphorylase promoter was added outside the clean deletion flanks to select for plasmid loss following the deletion event. The created plasmid is referred to as pMU1657, and is depicted in FIG. 23. The sequence of the pMU1657 plasmid is provided as SEQ ID NO:5.

### Creation of hpt Deletion

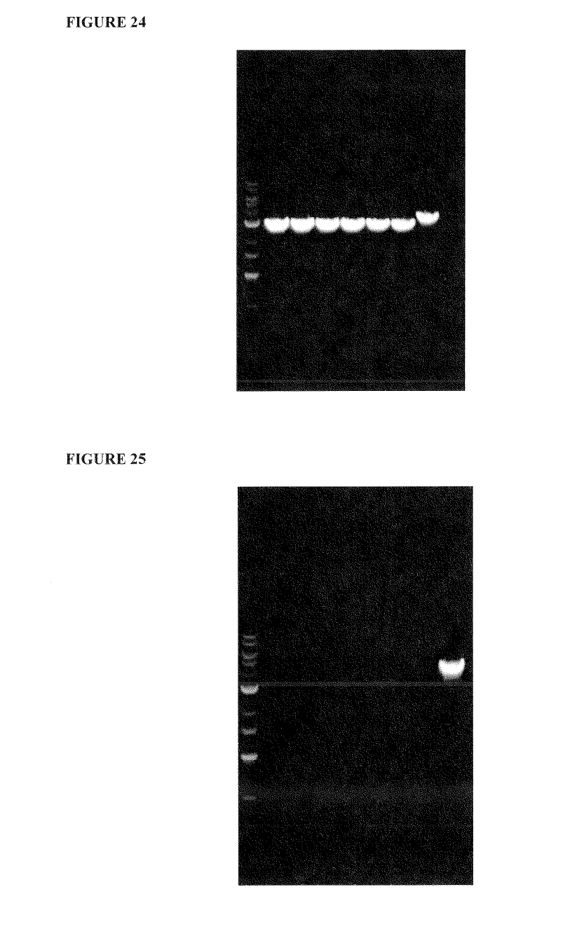

Plasmid pMU1657 was transformed into the wild type C. thermocellum strain 1313 and selected for on chloramphenicol. Several colonies were selected and the transformed plasmid was verified by PCR. A colony was diluted into C. thermocellum growth medium and grown overnight. The following morning the dilution closest to an O.D of ˜1.0 was serially diluted and plated on defined medium containing 500 ug/ml 8-azahypoxanthine. Two days later, colonies were observed on the dilution representing 10⁻³. Six colonies were selected and PCR screens were performed to verify that the hpt gene had been deleted from the chromosome. After PCR amplification, the expected size of the wild type product having no deletion is 3380 bp and the expected size of the hpt deletion is 2820 bp. As shown in FIG. 24, the hpt gene in all six selected colonies was deleted, as indicated by size of the amplified fragment. The colonies were subsequently screened by PCR amplification to confirm that the plasmid had been cured (or lost). In FIG. 25, lane (P) represents the plasmid control that is 3750 bp in size. Colonies that have lost the plasmid would have no product migrating in the lane. The lack of product in lanes 1-6 representing the six different colonies confirm that the plasmid had been cured.

The molecular data clearly shows that the hpt gene was successfully deleted and that the strain was plasmid free. To verify this, the new strain was grown up overnight and plated on thiamphenicol. After 5 days, no thiamphenicol resistant colonies were observed, further confirming the molecular data indicating plasmid loss. This data strongly suggests that hpt can be used as a negative selectable marker in C. thermocellum.

### Complementation

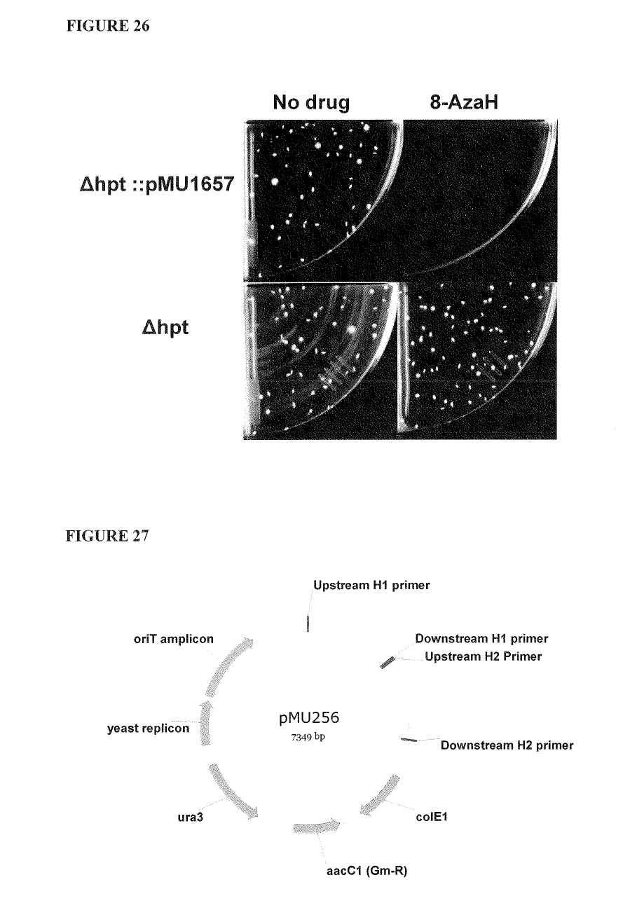

To provide stronger support for this conclusion, the knockout plasmid was reintroduced to look for complementation of the phenotype. Plasmid pMU1657 was transformed and the same plating procedure was used to assay for complementation and/or insensitivity to 8-azahypoxanthine. Results were exactly as expected. The Δhpt strain was completely insensitive to the drug and the strain containing the complementing plasmid showed sensitivity comparable to wild type levels (FIG. 26).

### Example 5

### Creation of a T. saccharolyticum hpt Knockout

To create a deletion of the hpt gene in T. saccharolyticum, the deletion vector pMU256 was built and transformed into T. saccharolyticum cells. The pMU256 plasmid is depicted in FIG. 27. PCR analysis, as shown in FIG. 28, indicates that that the hpt gene was successfully deleted. Complementation experiments were performed to determine whether the hpt deletion can be rescued.

### Example 6

### Use of hpt as a Positive Selectable Marker

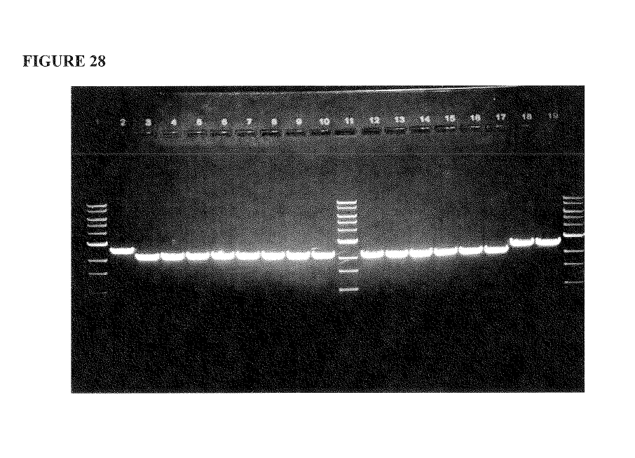

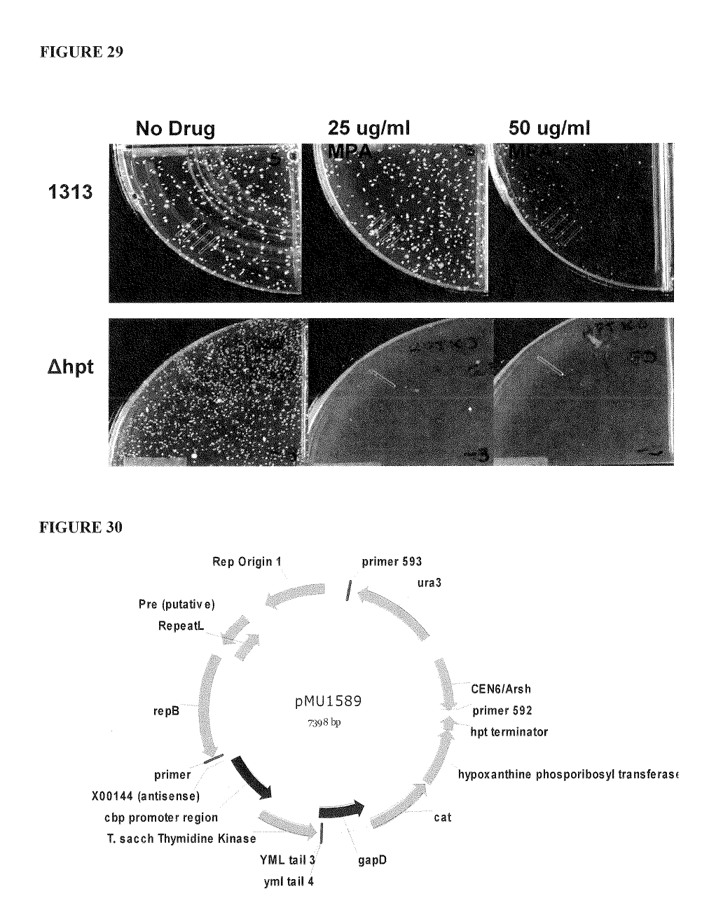

To determine if hpt can used as a positive selectable marker, both C. thermocellum strain 1313 and the Δhpt strain were grown to an OD ˜1.0 and plated on several concentrations of mycophenolic acid. Results of these experiments are shown in FIG. 29.

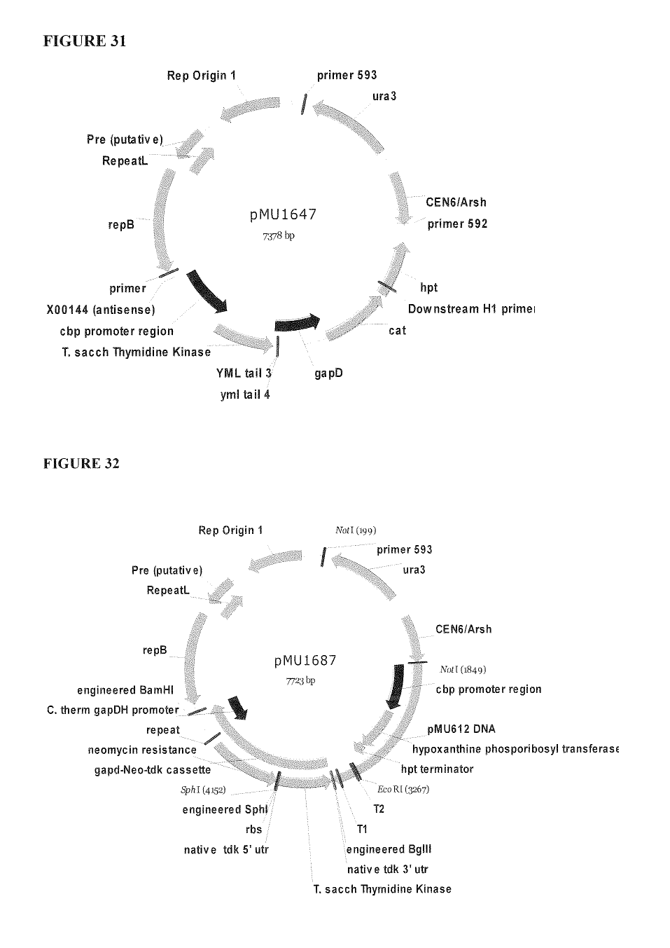

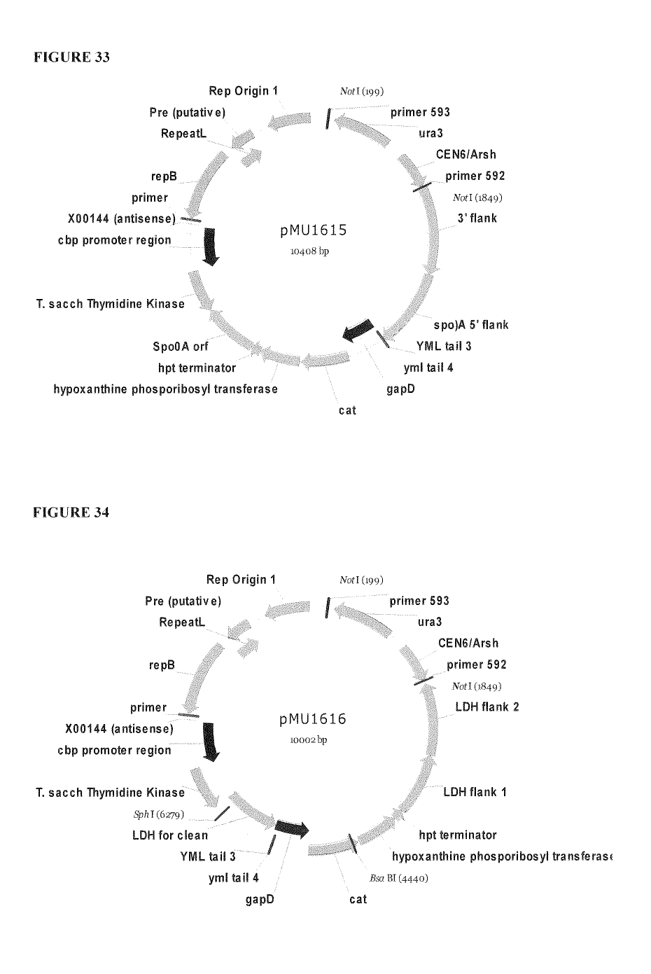

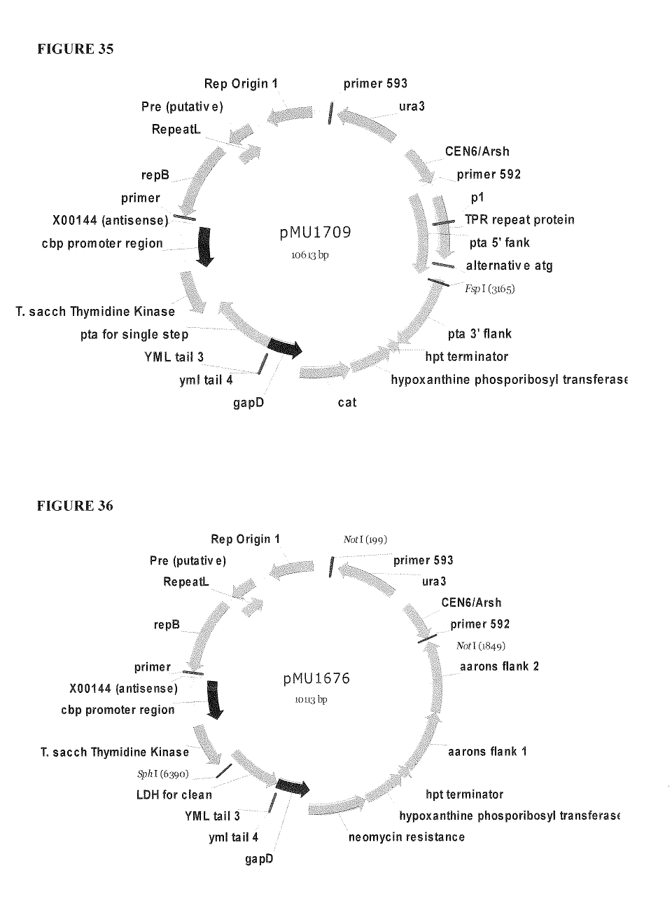

FIGS. 30-36 show several vectors which illustrate the principle of the invention. Several of these figures show vectors that do not contain any flanking sequence homologous to a targeted gene. A separate set of actual targeting gene deletion vectors are also represented. The vector maps shown in FIGS. 31-36 are non-limiting examples of the types of vectors that can be utilized to genetically engineer thermophilic organisms according to the invention.

### Example 7

### Use of a Genetic Tool to Make a Clean Deletion of ldh

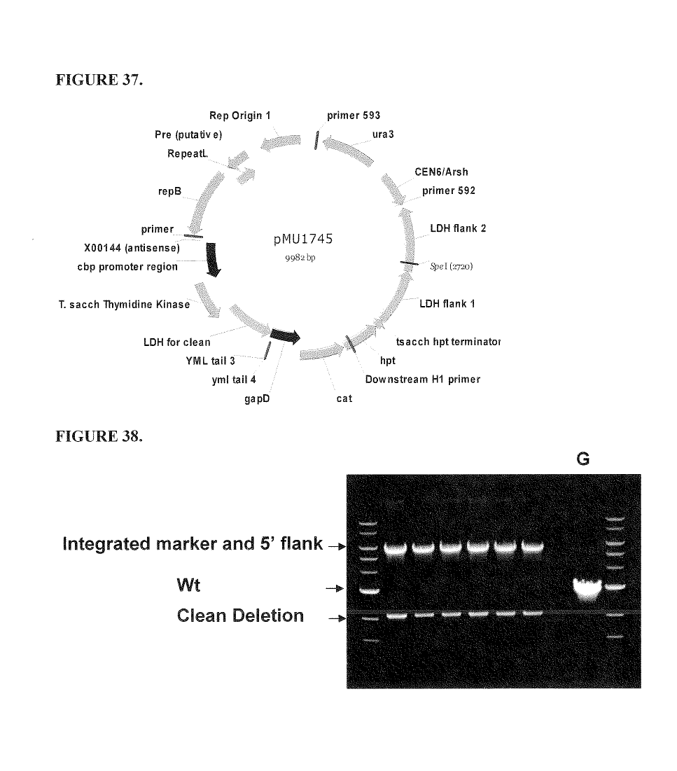

Plasmid pMU1745 is a replicating vector designed and constructed to make in-frame clean deletions of the lactate dehydrogenase (ldh) gene (FIG. 37). This vector was transformed into C. thermocellum and selected for in liquid growth media containing 6 ug/ml thiamphenicol. The transformed culture was subsequently plated on solid growth media containing 20 ug/ml thiamphenicol and 10 ug/ml FUDR and colonies were observed after ˜48 hours. Six colonies were PCR screened using primers flanking the ldh locus. Integration of the entire cat-hpt operon and the duplicated upstream region was observed based on the 5.8 kb PCR product observed (FIG. 38).

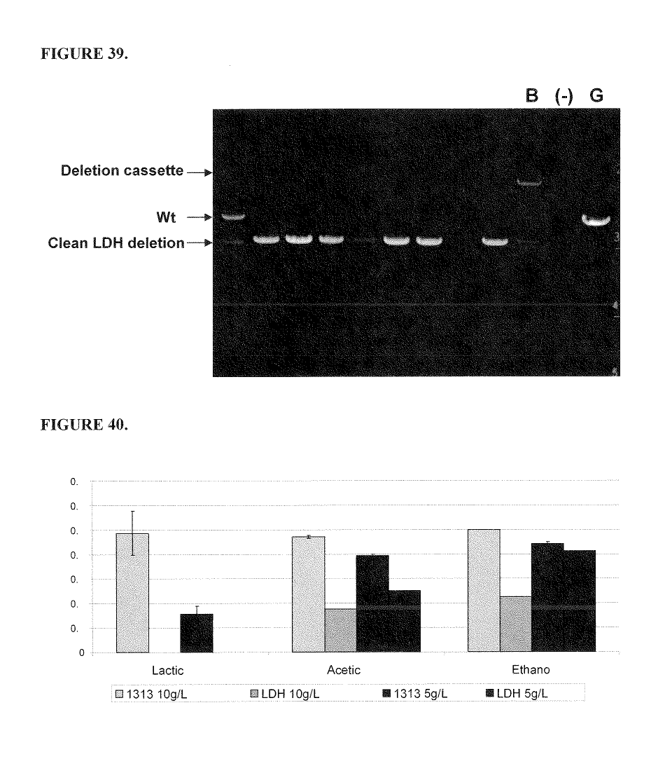

One colony of the selected colonies described above was inoculated into C. thermocellum growth media and the following morning a dilution series of this culture was plated on minimal media containing 500 ug/ml 8-azahypoxanthine. Eight colonies were PCR screened using primers flanking the LDH locus and a band of ˜2.1 kb representing the clean deletion was observed in seven out of eight colonies (FIG. 39).

To further confirm that lactate deydrogenase was deleted from the genome, two batch fermentations were run containing 5 g/l cellobiose and 5 g/l avicel respectively. In both fermentations, no lactate was made by the Δldh strain as shown in FIG. 40.

### Example 8

### Creation of a Marked Mutation

Primer design for amplification of DNA from C. thermocellum 1313 was based on the available C. thermocellum ATCC 27405 genome (http://genome.jgi-psf.org/cloth/cloth.home.html). The oligonucleotides and the plasmids/strains used in this study are listed in Table 1. The 5′ and 3′ flanking regions (˜1 kb) of pyrF and pta were amplified and assembled using yeast gap repair cloning to create gene deletion plasmids. (Shanks et al. Appl Environ Microbiol 11:5027-5036. (2006)).

TABLE 1
Plasmids and strains used.

| Plasmids/ | | Source or |
|---|---|---|
| strains | Description and relevant characteristics | references |
| Plasmids | | |
| pUC19 | General purpose cloning vector, Ap | NEB¹ |
| pMU102 | pMU104, region between the FokI and EcoRI sites has been | This study |
| | deleted, Cm | |
| pMU104 | pNW33N, E. coli-C. thermocellum shuttle vector, Cm | BGSC² |
| pMU110 | pMQ87, S. cerevisiae.-E. coli shuttle vector, Ura+, Gm | This study |
| pMU111 | pMU110 with aac1 (Gm) replaced by cat from pMU104, Ura+, | This study |
| | Cm | |
| pMU113 | pMU111 with C. the. gapDHp driving cat, Ura+, Cm | This study |
| pMU245 | E. coli-C. thermoceelum cloning vector, Ap | This study |
| pMU357 | S. cerevisiae-E. coli-C. thermocellum shuttle vector for | This study |
| | expressing genes in C. the., C. the gapDHp, Ura+, Cm | |
| pMU440 | S. cerevisiae-E. coli-C. thermocellum shuttle vector, C. therm | This study |
| | ΔpyrF cassette, Ura+, Cm | |
| pMU482 | E. coli-C. thermocellum shuttle vector, C. the ΔpyrF cassette, | This study |
| | Cm | |
| pMU582 | pMU110, C. the. cbp promoter, cbp gene, T1T2 terminator, | This study |
| | Ura+, Gm | |
| pMU597 | pMU582, C. the cbp gene replaced by C. the. pyrF gene- | This study |
| | creating cbpp-pyrF cassette, Ura+, Gm | |
| pMU612 | pMU102 containing cbpp-pyrF cassette, Cm | This study |
| pMU749 | pMU245, CEN6/ARSH4, ura3-S. cer.-E. coli-C. the. vector, | This study |
| | Ura+, Ap | |
| pMU769 | pMU749 with ΔpyrF::gapDHp-cat cassette, Ura+, Ap, Cm | This study |
| pMU1016 | pMU749 with Δpta::gapDHp-cat cassette, Ura+, Ap, Cm | This study |
| pMU1162 | pMU1016 with cbpp-pyrF cassette, Ura+, Ap, Cm | This study |
| Strains | | |
| E. coli | Top10 cloning strain | Invitrogen³ |
| S. cereviciae | InvSC1 Ura3⁻ for gap repair cloning | Invitrogen³ |
| C. thermocellum | M0003 Wild type C. thermocellum DSM 1313 | DSMZ⁴ |
| | M0970 C. thermocellum DSM 1313 ΔpyrF | This study |
| | M0971 C. thermocellum DSM 1313 ΔpyrF Δpta::gapDHp-ca | This study |
| | M1061 C. thermocellum DSM 1313 ΔpyrF pMU612 | This study |
| | M1062 C. thermocellum DSM 1313 ΔpyrF pMU102 | This study |

¹New England Biolabs, Ipswich, MA
²Bacillus Genetic Stock Center, http://www.bgsc.org/
³Invitrogen, Carlsbad, California
⁴Deutsche Sammlung von Mikroorganismen and Zellkulturen GmbH, Germany

The pyrF and pta deletion vectors (pMU769 and pMU1162, respectively) contained cat (chloramphenicol acetyl transferase) expressed from the C. thermocellum gapDH promoter (gapDHp) positioned between the 5′ and 3′ flanking regions. The pyrF complementing construct (pMU612) contained pyrF expressed from the C. thermocellum cellobiose phosphorylase (cbp) promoter (cbpp). All DNA manipulations and cloning procedures were performed as per Maniatis et al.

For this transformation protocol a pulse generator was custom built and utilized a solid-state insulated-gate bipolar transistor (IGBT) instead of a power tetrode, as the high voltage switch (Infineon, part no. FZ200R65KF2). The device was charged with a high-voltage power supply from Emco (part no. F101). The charge was stored in an 8 kV 32 capacitor made by General Atomics (part no. 39742). Pulse duration and interval was controlled by an arbitrary function generator (Tektronix, part no. AFG3101). All manipulations were done under anaerobic conditions. Cultures were grown to mid-log phase (OD₆₀₀=0.4-0.8) in rich medium and harvested by centrifugation (2200×g for 12-14 min). Cells were washed twice in autoclaved, deionized water and the final pellet was resuspended in 200 μl deionized water. For each transformation, 20 μl of cell suspension was added, along with 1-8 μg of plasmid DNA, to a 0.1 cm gap electorporation cuvette (Fisher Scientific).

A series of 60 square pulses were applied to the sample. The period of the pulses was 300 μs and the amplitude was 1.9 kV, resulting in an applied field strength of 19 kV/cm. After pulsing, cells were recovered overnight (15-18 h) at 51° C. in 3-5 ml rich medium. For liquid selection, recovered cultures were inoculated (10% v/v) into either rich medium supplemented with 3-6 μg/ml thiamphenicol (Tm) or uracil-free MJ medium when selecting for uracil prototrophy. For selecting transformants on solid medium, the recovery cultures were plated as agar suspensions in rich medium with 3-6 μg/ml Tm or MJ medium. To select pyrF mutants, transformants were grown in 3 μg/ml Tm. The cultures were then diluted to approximately 10⁸ cells/ml and 100 μl of the diluted culture was plated as agar suspensions in rich medium containing 5-FOA. 5-FOA resistant colonies were screened by PCR using primers, which anneal outside of the regions of homology used to delete pyrF. The pyrF::gapDHp-cat mutants were isolated and the same primer set was used to screen pyrF::gapDHp-cat mutants.

To select pta::gapDHp-cat mutants, the ΔpyrF strain transformed with pMU1162 was gown in 5 ml of rich medium supplemented with 6-12 μg/ml Tm or in MJ medium for about 14-16 hours. Various volumes of the cultures (ranging from 100 to 1 ml) were plated as agar suspensions of rich medium containing 5-FOA and 48 μg/ml Tm. Resistant colonies were screened by PCR using primers which anneal outside of the regions of homology used to delete pta.

In order to create a marked mutation, a positive selection was needed to select for a chromosomal integration event and a negative selection was needed to select for loss of the replicating knock out plasmid. The latter component can be achieved using the ΔpyrF strain and ectopic expression of pyrF from a plasmid. To achieve the former, the cat marker, which provides Tm resistance at thermophilic temperatures from a multi-copy plasmid, was used for its ability to provide Tm resistance when harbored in single copy on the chromosome at the pyrF locus. An allelic replacement vector was constructed, pMU769, to delete the pyrF gene and replace it with cat controlled by the native glyceraldehyde 3-phosphate dehydrogenase (gapDH) promoter of C. thermocellum. The vector contained gapDHp-cat elements positioned between 5′ and 3′ pyrF flanking DNA. To replace pyrF with gapDHp-cat, C. thermocellum transformants containing pMU769 were subjected to two simultaneous selections in liquid, rich medium. Thiamphenicol was used to select for the plasmid encoded gapDHp-cat, while 5-FOA was used to select against chromosomal pryF. Recovered cultures were evaluated by PCR using primers that anneal upstream and downstream of pyrF. Using these conditions, replacing the pyrF gene with gapDHp-cat increased the PCR amplicon size by ˜300 bp as compared to the wt. This result demonstrated that cat expressed from the gapDH promoter was functional in a single copy on the C. thermocellum chromosome and could be used as marker for allele replacement.

### Example 9

### Deletion of the C. thermocellum pta Gene Using pyrF and cat Selection

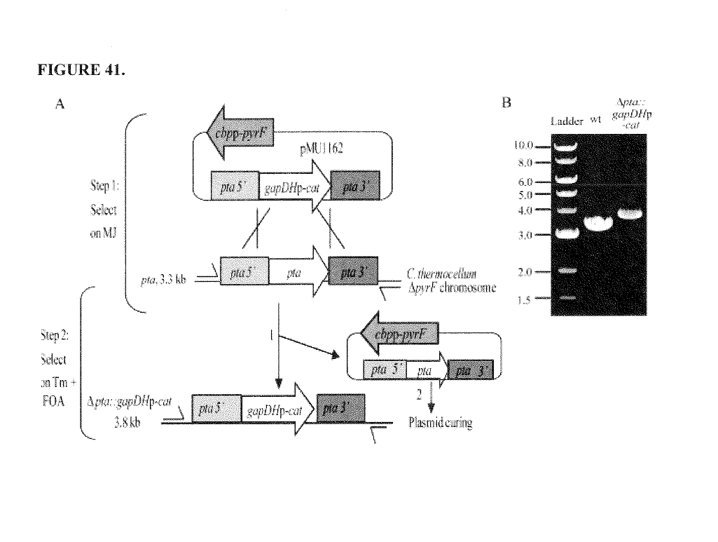

Mixed acid fermentation of C. thermocellum involves co-production of lactic acid, acetic acid, formic acid, and ethanol. For C. thermocellum strain DSM 1313 acetic acid one co-product that needs to be eliminated to create a strain with increased ethanol yield. The production of acetic acid from acetyl-CoA involves two enzymatic activities that are catalyzed by Pta and Ack. The scheme used to replace pta with cat expressed from the gapDH promoter in the C. thermocellum pyrF background is shown in FIG. 41A. MJ medium lacking uracil was used to select ΔpyrF clones restored to uracil prototrophy as a result of being transformed with pMU1162 (FIG. 41A step 1). Single colonies representing transformants were propagated in liquid medium with Tm selection prior to plating on Tm plus 5-FOA (FIG. 41A Step 2). Resistant colonies were screened by PCR using primers that anneal outside of the regions of homology used to delete pta (FIG. 41A). Clones in which pta was replaced by the gapDHp-cat cassette were discernable by a 0.5 kb increase in the size of the amplicon. For simplicity the Δpta mutants generated in the pyrF background strain are designated as pta::gapDHp-cat strain from here after (excluding the background strain pyrF genotype). The expected amplicon was 3.3 kb for wt and 3.8 kb for the pta::gapDHp-cat strain (FIGS. 41A and B). The pta locus was sequenced to confirm allele replacement. Batch fermentations in anaerobic tubes with wild-type, ΔpyrF, and pta::gapDHp-cat were performed at 55° C. in rich medium under a nitrogen atmosphere utilizing cellobiose or Avicel as the primary carbon source. The fermentation products were analyzed using high-performance liquid chromatography (HPLC) as previously described. (Shaw et al. Proc Natl Acad Sci. 105:13769-13774. (2008)).

Growth rate measurements were performed in a 200 μl volume in a 96-well plate at 55° C. The optical density at 600 nm was read by a Powerwave XS platereader customized by the manufacturer to incubate up to 68° C. (BioTek). The plates were shaken continuously and read at three minute intervals. Each sample was measured in quadruplicate. The specific growth rate (0 was determined by measuring the slope of the natural log-transformed OD readings. A two hour sliding window of OD readings between 0.08 and 1.00 were used for determination of maximum rate μmax. In all cases, the R-squared value was greater than 0.99.

The growth of the pta::gapDHp-cat strain was compared to the wt and ΔpyrF strains in rich medium, with and without uracil supplementation. Although initial rates of growth of the ΔpyrF and wt strains were similar, the ΔpyrF strain slowed abruptly at an OD of ˜0.7, while the wt continued to grow until it reached an OD of ˜1.6, suggesting that the rich medium was uracil-limited. Supplementing the medium with an additional 40 μg/ml uracil eliminated the growth defect of the ΔpyrF strain and resulted in a growth curve that was indistinguishable from the wt strain. Even with additional uracil supplementation to compensate for the ΔpyrF mutation, the maximum specific growth rate (μmax) of the pta::gapDHp-cat strain was about one third lower than that of either the wt or the ΔpyrF strains and the final OD was also reduced. This indicates that the growth defect of the pta::gapDHp-cat strain is distinct from the growth defect of the ΔpyrF strain and is a result of the pta mutation. End product analysis was performed on batch fermentations started at pH 7.0 with 5 g/l cellobiose as the primary carbon source under anaerobic conditions with a nitrogen atmosphere and 80 ml working volume. After 48 hours of fermentation the wt and ΔpyrF strain produced about 1 g/l of acetic acid whereas the acetic acid production of the pta::gapDHp-cat was indistinguishable from background levels (average 0.03 g/l). All three strains produced comparable amounts of ethanol and lactic acid. Due to the growth defect of the pta::gapDHp-cat strain a 96 hour sample point was taken but acetate levels did not change, measuring 0.031 g/l. The average dry cell mass for wt, ΔpyrF, and pta::gapDHp-cat strains were 0.54 g, 0.54 g and 0.35 g, respectively indicating that the pta::gapDHp-cat strain made about one-third less biomass compared to the wt and ΔpyrF strain.

1. A vector for use in an anaerobic thermophilic host comprising:
(a) one or more selectable marker sequences, wherein each selectable marker sequence comprises a nucleic acid sequence encoding for a positive and/or negative selectable marker; and
(b) a thermophilic host sequence, wherein said thermophilic host sequence comprises a nucleic acid sequence that is endogenous to said thermophilic host.

2. The vector of claim 1, wherein said selectable markers are selected from the group consisting of thymidine kinase (tdk), hypoxanthine phosphoribosyltransferase (hpt), orotidine-5′-phosphate decarboxylase (pyrF), chloramphenicol acetyltransferase (cat), neomycin (neo), and kanamycin (kan).

3. The vector of claim 1, comprising at least one positive selectable marker sequence.

4. The vector of claim 1, comprising at least one negative selectable marker sequence.

5. The vector of claim 1, comprising at least two selectable marker sequences.

6. The vector of claim 1, comprising at least one positive selectable marker sequence and at least one negative selectable marker sequence.

7. The vector of claim 1, wherein one of said selectable marker sequences encodes for a selectable marker that provides for both positive and negative selection.

8. The vector of claim 1, wherein one of said selectable markers is hpt.

9. The vector of claim 1, wherein one of said selectable markers is tdk.

10. The vector of claim 1, comprising one selectable marker sequence encoding tdk and one selectable marker sequence encoding for hpt.

11. The vector of claim 9, wherein said tdk is from Thermoanaerobacterium saccharolyticum.

12. The vector of claim 1, wherein one of said selectable marker sequences is pyrF.

13. The vector of claim 1, wherein said thermophilic host sequence comprises nucleic acid sequences of regions flanking an endogenous target gene.

14. The vector of claim 13, wherein said endogenous target gene is lactate dehydrogenase (ldh), hydrogenase, phosphotransacetylase (pta), acetate kinase (ack), nitrogenase, pyruvate formate lyase (pfl), methylglyoxal synthase, Spo0A or a gene involved in central metabolism, stress response or carbohydrate utilization.

15. The vector of claim 1, wherein said thermophilic host sequence comprises a nucleic acid sequence of an endogenous replicon, an endogenous origin of replication, or an endogenous regulatory sequence.

16. The vector of claim 1, further comprising one or more cellulase sequences, wherein each of said cellulase sequences comprises a nucleic acid sequence encoding for a heterologous cellulase, a heterologous xylose isomerase, a xylulokinase, or a xylulokinase associated transporter.

17. The vector of claim 16, wherein said heterologous cellulase is an endoglucanase, a β-glucosidase or an exoglucanase.

18. The vector of claim 1, wherein said anaerobic thermophilic host is selected from the group consisting of Clostridium thermocellum, Thermoanaerobacterium saccharolyticum, Thermoanaerobacter ethanolicus (JW200 DSM 2246), Thermoanaerobacterium thermosaccharolyticum sp. (M0523), Thermoanaerobacterium thermosaccharolyticum sp. (M0524), Thermoanaerobacterium aotearoense (DSM 10170), Thermoanaerobacterium thermosaccharolyticum (HG-8 ATCC 31960), Thermoanaerobacterium saccharolyticum (B6A), Thermoanaerobacterium saccharolyticum (B6A-RI ATCC 49915), Thermoanaerobacterium thermosaccharolyticum sp. (M0795), Thermoanaerobacterium xylanolyticum (DSM 7097), Thermoanaerobacterium thermosaccharolyticum (ATCC 7956), Thermoanaerobacter pseudoethanolicus (39E ATCC 33223), and Thermoanaerobacter brockii (ATCC 35047).

19. The vector of claim 1, wherein said anaerobic thermophilic host is a xylanolytic host of the genus Anaerocellum, Caldicellulosiruptor or Moorella.

20. A vector for use in an anaerobic thermophilic host comprising:
(a) one or more selectable marker sequences, wherein said selectable marker sequences are selected from the group consisting of hpt and tdk; and
(b) a thermophilic host sequence, wherein said thermophilic host sequence comprises a nucleic acid sequence that is endogenous to said thermophilic host.

21. The vector of claim 20, comprising at least one positive selectable marker sequence.

22. The vector of claim 20, comprising at least one negative selectable marker sequence.

23. The vector of claim 20, comprising at least two selectable marker sequences.

24. The vector of claim 20, wherein said tdk is from Thermoanaerobacterium saccharolyticum.

25. The vector of claim 20, further comprising the selectable marker pyrF, chloramphenicol acetyltransferase (cat), neomycin (neo) or kanamycin (kan).

26. The vector of claim 20, wherein said thermophilic host sequence comprises nucleic acid sequences of regions flanking an endogenous target gene.

27. The vector of claim 26, wherein said endogenous target gene is lactate dehydrogenase (ldh), hydrogenase, phosphotransacetylase (pta), acetate kinase (ack), nitrogenase, pyruvate formate lyase (pfl), methylglyoxal synthase, Spo0A or a gene involved in central metabolism, stress response or carbohydrate utilization.

28. The vector of claim 20, wherein said thermophilic host sequence comprises a nucleic acid sequence of an endogenous replicon, an endogenous origin of replication, or an endogenous regulatory sequence.

29. The vector of claim 20, further comprising one or more cellulase sequences, wherein each of said cellulase sequences comprises a nucleic acid sequence encoding for a heterologous cellulase.

30. The vector of claim 20, wherein said thermophilic host is selected from the group consisting of Clostridium thermocellum, Thermoanaerobacterium saccharolyticum, Thermoanaerobacter ethanolicus (JW200 DSM 2246), Thermoanaerobacterium thermosaccharolyticum sp. (M0523), Thermoanaerobacterium thermosaccharolyticum sp. (M0524), Thermoanaerobacterium aotearoense (DSM 10170), Thermoanaerobacterium thermosaccharolyticum (HG-8 ATCC 31960), Thermoanaerobacterium saccharolyticum (B6A), Thermoanaerobacterium saccharolyticum (B6A-RI ATCC 49915), Thermoanaerobacterium thermosaccharolyticum sp. (M0795), Thermoanaerobacterium xylanolyticum (DSM 7097), Thermoanaerobacterium thermosaccharolyticum (ATCC 7956), Thermoanaerobacter pseudo ethanolicus (39E ATCC 33223), and Thermoanaerobacter brockii (ATCC 35047).

31. The vector of claim 20, wherein said anaerobic thermophilic host is a xylanolytic host of the genus Anaerocellum, Caldicellulosiruptor or Moorella.

32. A thermophilic host cell comprising the vector of claim 1.

33. The thermophilic host cell of claim 32, wherein the endogenous hpt gene of said host cell has been deleted (Δhpt).

34. The thermophilic host cell of claim 32, wherein the endogenous tdk gene of said host cell has been deleted (Δtdk).

35. The thermophilic host cell of claim 32, wherein the endogenous pyrF gene of said host cell has been deleted (ΔpyrF).

36. The thermophilic host of claim 32, wherein said host is not auxotrophic.

37. A method for producing a transformed anaerobic thermophilic host cell, said method comprising the following steps:
(a) transforming said thermophilic host cell with the vector of claim 1; and
(b) selecting said host cell for the presence of said vector within the host cell.

38. A method of making an unmarked anaerobic thermophilic host cell, said method comprising the following steps:
(a) transforming said thermophilic host cell with the vector of claim 1;
(b) selecting said host cell for the presence of said vector within the host cell;
(c) culturing said host cell for a length of time and under conditions whereby the vector replicates; and
(d) selecting said host cell for the absence of said vector within the host cell.

39. A method of making one or more targeted gene deletions in an anaerobic thermophilic host cell, said method comprising the following steps:
(a) transforming said thermophilic host cell with the vector of claim 1, wherein said vector comprises thermophilic host sequence flanking an endogenous target gene;
(b) selecting said host cell for the presence of said vector within the host cell;
(c) culturing said host cell for a length of time and under conditions whereby homologous recombination occurs between the vector and the host cell genome; and
(d) determining whether said target gene has been deleted; and, optionally,
(e) repeating steps (a)-(d) for deletion of a different target gene.

40. The method of claim 39, wherein said target gene encodes for lactate dehydrogenase (ldh), hydrogenase, phosphotransacetylase (pta), acetate kinase (ack), nitrogenase, pyruvate formate lyase (pfl), methylglyoxal synthase, Spo0A or a gene involved in central metabolism, stress response or carbohydrate utilization.

41. A method for recycling genetic markers in an anaerobic thermophilic host cell, said method comprising the following steps:
(a) transforming said thermophilic host cell with the vector of claim 1;
(b) selecting said host cell for the presence of said vector within the host cell;
(c) culturing said host cell for a length of time and under conditions whereby the vector replicates; and
(d) selecting said host cell for the absence of said vector within the host cell; and, optionally,
(e) repeating steps (a)-(d).

42. A thermophilic host cell produced by the method of claim 37.
